(19) United States
(12) Patent Application Publication
(10) Pub. No.: US 2021/0370053 A1
(43) Pub. Date: Dec. 2, 2021

(54) Neuro-stimulation and Sensor Devices Comprising Low-Impedance Electrodes, and Methods, Systems And Uses Thereof

(71) Applicant: Newrom Biomedical, LLC, San Diego, CA (US)

(72) Inventors: Sungho Jin, San Diego, CA (US); Krishnan Chakravarthy, San Diego, CA (US); Chulmin Choi, San Diego, CA (US); Kyungjun Hwang, San Diego, CA (US)

(21) Appl. No.: 17/300,256

(22) PCT Filed: Oct. 30, 2019
(86) PCT No.: PCT/US2019/058969

§ 371 Date: Apr. 30, 2021

Related U.S. Application Data
(60) Provisional application No. 62/903,946, filed on Sep. 23, 2019.
(60) Provisional application No. 62/752,356, filed on Oct. 30, 2018.
(60) Provisional application No. 62/882,523, filed on Aug. 4, 2019.
(60) Provisional application No. 62/819,682, filed on Mar. 18, 2019.

(51) Int. Cl. A61N 1/05; A61N 1/36; A61B 5/00

(52) U.S. Cl. CPC A61N 1/0553; A61N 1/36071; A61N 1/36062; B82Y 5/00; A61B 5/6877; A61B 2562/125; A61N 1/0556

(57) ABSTRACT

Disclosed are platforms to enable lower impedance electrode array, together with a miniaturized battery pack. Lower impedance can be achieved by different approaches, according to the invention, including surface modifications, preferably in nanoscale. Also disclosed are articles and control systems comprising medical implant neural stimulator devices, neural diagnosis tools, spinal cord and peripheral nerve stimulations, and cochlear implants. More particularly, the invention discloses means for reducing pains in human body, utilizing innovative components and systems comprising an epidural lead having multiple electrodes at a distal end, the electrodes being configured in an array and being selectable to provide either unilateral or bilateral neural stimulation. In an example, advanced spinal cord stimulation (SCS) electrodes having pre-designed novel, metallic or non-metallic nanostructured surface with desirable high-aspect-ratio nanopillar features for superior neural electrode functionality exhibiting significantly reduced electrical impedance are disclosed.

### CROSS-REFERENCE TO RELATED APPLICATIONS

This patent document claims benefit of priority of U.S. Provisional Patent Application No. 62/752,356, entitled “SURFACE MODIFIED NEURO-STIMULATION ELECTRODE ARRAY, PSEUDO-PHYSIOLOGICAL PERFORMANCE, AND METHODS, DEVICE SYSTEMS AND APPLICATIONS” filed on Oct. 30, 2018, U.S. Provisional Patent Application No. 62/882,523, entitled “NEURO-STIMULATION SYSTEM INCLUDING LOW IMPEDANCE STRUCTURES, COMPLIANT, GAP-REDUCIBLE ELECTRODE ARRAYS, FABRICATION METHODS, AND USES” filed on Aug. 4, 2019, U.S. Provisional Patent Application No. 62/819,682, entitled “ENHANCED NEURO-STIMULATION AND FEEDBACK-SENSING ELECTRODE ARRAY, FABRICATION METHODS, DEVICES AND USES” filed on Mar. 18, 2019, and U.S. Provisional Patent Application No. 62/903,946 entitled “IMPROVED NEURO-STIMULATION SYSTEM INCLUDING PRE-SURFACE-CONTROLLED LOW IMPEDANCE STRUCTURES, METHODS, AND USES” filed on Sep. 23, 2019. The entire contents of the aforementioned patent applications are incorporated by reference as part of the disclosure of this patent document.

### TECHNICAL FIELD

This disclosure relates to devices, systems, and methods for filtering smoke.

### BACKGROUND

Neuro-stimulation implant devices are useful for control of human activities including spinal cord stimulation devices for pain reduction. Electrical signaling between neurons in human and animal nervous systems is part of the fundamental operating characteristic, which is linked to some of the most tragic and widespread health and disease conditions in our society. For example, Alzheimer's Disease, heart disease, hearing loss and head trauma, epilepsy, chronic pains, are all related to neural misfiring, insufficiencies, and/or dysfunction. Unfortunately, the regeneration and reconnection of damaged neuronal pathways naturally or with surgery and medication is limited. Therefore, nerve damage from disease, genetic disorders, or trauma is often permanent and life threatening. However, a combination of nanotechnology and biomaterials for small implantable electrodes can offer a means to enable sending/receiving electrical signals, normally only possible between healthy nerves.

An important and emerging area of neural stimulation is the field of pain management, which is becoming national public health issues because of the growing need for chronic pain management and the risks of opioid use and misuse. Pain is one of the oldest challenges for medicine, and despite some advanced understanding of its pathophysiology, chronic pain continues to burden many patients. It is therefore highly desirable to develop alternative techniques for pain relief, such as neural stimulation based approaches which demonstrate effectiveness in pain reduction. Also see U.S. Pat. No. 5,417,719 by V. W. Hull, et al, “Method of Using a Spinal Cord Stimulation Lead”, issued on May 23, 1995, U.S. Pat. No. 5,766,527 by G. R. Schildgen et al, “Method of Manufacturing Medical Electrical Lead”, issued on Jun. 16, 1998, US Patent Application No. US 2013/0110196A1 by K. Alataris, et al, “Selective High Frequency Spinal Cord Stimulation For Inhibiting Pain With Reduced Side Effects, and Associated Systems and Methods”, published on May 2, 2013.

While electrical stimulation principles has been used for decades in treating chronic neuropathic pain, spinal cord stimulation (SCS) for neuro function modulation has become one of the most exciting recent developments in the field of chronic pain management. Sensory neurons (nerve fibers in the spinal cord) that carry nerve impulses from sensory stimuli towards the central nervous system and brain, can be stimulated by electrical signals to inhibit chronic pain. Chronic Pain is one of the leading causes for physical and emotional suffering as well as disruption of family life and societal functions, and hence is receiving much attention from medical and societal perspectives.

Electrical stimulation does not eliminate the source of pain, but rather it simply interferes with the neural pain signal to the brain. The principle of spinal cord stimulator is via masking of neural pain signals before they reach the brain, by intentionally delivering electric pulses to electrodes placed over the spinal cord to modify the pain signals so that they are either not perceived or are replaced by a different (e.g., tingling) sensation. While the amount of pain relief varies for each person, a typical desired goal for spinal cord stimulation is at least 50% reduction in pain. Low-frequency current is generally utilized to replace the pain sensation with a tingling type feeling (paresthesia feeling). High-frequency electrical current signals or burst pulse signals are utilized to substantially mask the pain.

Neural electrodes are critical components for electrical stimulation as well as neural signal recording. The human nervous system essentially controls all body functions including sensing/hearing of outside stimulus to the human body and needed body response with actuation or movement, as well as triggering of automatic impulses such as breathing. Disorders in the neural system often arise due to the damaged connections within the network of neurons, or due to the insufficient secretion of neurochemicals at the desired locations. In order to mitigate these problems, it is desirable to develop advanced technologies to control/modulate human neural function. As the basis of neural function is to send and receive electrical signals, a reliable interfacing via robust electrodes is required between the neural cells and electronics that may sit within or outside of the nervous system.

The quality of the neuron-to-electronics interface depends on the safety, reliability and efficiency of the electrode. It is essential to design the electrode material so that it is resistant to biofouling and inflammation and is capable of maximizing neural signal collection or actuation signal delivery with low impedance characteristics. However, the effectiveness of neural electrode interfacing technology has been severely limited due to the biofouling effect of cellular growth on the surface of implanted electrodes. The growth of endothelial or glial cells on the surface of a biocompatible implanted devices is a normal biological process, and for many implants is regarded as essential for successful integration into the body. In the case of neural electrodes, however, cellular growth on the implant surface is detrimental to the overall function of the electrode.

For example, the presence of a sheath of cells (tissue encapsulation) on the electrode is a well known problem which reduces the signal strength and limits the radial distance the electrode is capable of sending and receiving electric signals. With greater control over the distance and direction, fewer and more accurate electrodes may be developed and incorporated into the body. Additionally, an electrode, unaffected by cellular biofouling may provide care to a larger age demographic, cut down on the number of replacement surgeries, and as result lower overall cost of neuromodulation treatments.

Many techniques have been attempted to minimize the biofouling effect. Topographical patterning can influence cell adhesion/migration/orientation, shape, and cell fate. Polymer coatings such as poly(dimethylsiloxane) (PDMS) and poly(ethylene glycol) (PEG) have also been used to minimize the interaction by providing a hydrophobic coating. However, a coating of electrode surface with a polymer tends to substantially increase the electrical impedance because of the insulating nature of such coating materials. It will be highly desirable if one can achieve a significant reduction of impedance or at least maintain the low level of impedance in spite of the addition of electrically insulating anti-biofouling coating on the electrode surface.

For implantable pulse generator (IPG) devices to be implanted inside human body, a surgery to open up the skin tissue is necessary. Typical spinal cord stimulators package includes electrode lead wires comprising an array of multiple electrodes and a battery pack to supply electrical energy for providing the desired pulse signals. The battery pack also incorporates some control circuits for pulsing.

Electrode impedance is one area where changes occurring at the electrode-tissue interface affect power usage. Electrode impedance can be described as the resistance to charge exchange between the electrode surface and the electrolyte. Power is directly proportional to electrode impedance, such that increases in electrode impedance result in increases in the device's power requirements.

This invention discloses a platform to enable such a beneficial lower impedance electrode array, together with a miniaturized battery pack. Lower impedance can be achieved by different approaches, according to the invention, such as (i) introducing electrode surface nanotexturing with nanowires, nanopillars, nanopores, or highly porouse surface for much increased surface area (such as Pt black, Pt—Ir black, Au alloys or other alloys with surface roughnesss, TiN coating on {Pt, Pt—Ir, MP35N, Au or other metallic or Si-base or carbon-base elongated/porous structures}), (ii) enabling positioning of the electrode lead wire in the epidural space closer to the target spinal cord location with a secured geometrical stability, (iii) preventing long-term biofouling and associated loss of electrical conductivity between the electrode and the spinal cord (Tissue growth around implanted electrodes, with protein and cells at least partially covering the surface of the electrode, increases electrode impedance and thus power usage also rises.) (iv) optionally reducing the electrical lead extension wire length by anchoring the battery at a position much higher than the current lower-hip region (which is made feasible because of the miniaturized dimension and weight of the battery pack), and (v) optionally utilizing the electrode material having a much lower electrical resistivity (e.g., Au or dispersion-hardened Au, with electrical resistivity ρ˜2.4 um·cm, or Pt with ρ˜10.6) than currently used Pt-10% Ir (with ρ˜25 um·cm) or MP35N alloy (35% Co-35% Ni-20% Cr-10% Mo in wt. %, having ρ˜103 um·cm). Both Pt—Ir and MP35N alloys are mechanically strong and resists undesirable plastic or elastic deformation under stress. If a lower electrical resistivity material such as Au, dispersion-hardened Au or Pt is to be utilized as the electrode material, the electrode structure needs to be mechanically protected so that the alloy electrode is not subjected to inadvertent deformation.

### SUMMARY

The following presents a summary to provide a basic understanding of one or more embodiments of the invention. This summary is not intended to identify key or critical elements or delineate any scope of the particular embodiments or any scope of the claims. Its sole purpose is to present concepts in a simplified form as a prelude to the more detailed description that is presented later.

This invention discloses a platform to enable lower impedance electrode array, together with a miniaturized battery pack. Lower impedance can be achieved by different approaches, according to the invention, including surface modifications, preferably in nanoscale.

The invention discloses articles and control systems comprising medical implant neural stimulator devices, neural diagnosis tools, spinal cord and peripheral nerve stimulations, and cochlear implants. More particularly, the invention discloses means for reducing pains in human body, utilizing innovative components and systems comprising an epidural lead having multiple electrodes at a distal end, the electrodes being configured in an array and being selectable to provide either unilateral or bilateral neural stimulation.

In one example aspect of the invention, advanced spinal cord stimulation (SCS) electrodes having pre-designed novel, metallic or non-metallic nanostructured surface with desirable high-aspect-ratio nanopillar features for superior neural electrode functionality exhibiting significantly reduced electrical impedance are disclosed. The impedance reduction is at least by 50%, at least by a factor of two, preferably at least by a factor of five.

In another example aspect of the invention, methods to further increase the nanopillar aspect ratio for reduced impedance are also disclosed. Medical implant electrode alloys including commonly utilized implant electrode alloys such as Pt, Pt-10% Ir or MP35N alloy (35% Co-35% Ni-20% Cr-10% Mo in wt. %), Co—Cr alloy, are processed into desired nano-configurations, according to the invention, to exhibit desirably reduced impedance as well as enhanced anti-biofouling characteristics.

In another aspect of the invention, such a reduced electrical impedance (less resistive loss of electricity at bio interfaces) allows the consumption of less electricity and a much longer time use of battery power for neural stimulation in the case of implanted battery pack arrangement. For example, if the impedance is decreased by a factor of 5, the battery power use could be reduced by as much as a factor of 5, which implies the size of the battery to be implanted for SCS application can be decreased to a more desirable, miniature form factor, with the size reduction as much as by a factor of 5. Miniaturized battery size implies improved ease of implanting the power source in human body, and if desired, a single incision operation can be pursued to implant both the stimulating/sensing leads and the battery/controller pack. Other forms of electrical energies besides the batteries, such as biofuel device, thermoelectric generation based on temperature difference in various parts of human body, motion-related electricity generation using piezoelectric or electromagnetic power generation can also be utilized with reduced power consumption for neuro-stimulation devices according to the invention.

In another aspect of the invention, the invention also discloses SCS methods that can utilize both low frequency regime stimulation, BURST stimulation and its derivates, as well as high frequency regime cord stimulation methods for reducing chronic or transient pains, with the latter utilized to achieve electrical stimulation without or with reduced paresthesia such as an abnormal sensation of tingling, pricking or numbness.

Another aspect of the invention is to enable feedback-controlled neural stimulation. When electrical pulse is applied, e.g., to achieve pain reduction in human and animal body, the neurons and associated cells respond and send out electrical response signal such as ECAP (Electrically Evoked Compound Action Potential) and other neuronal signals. As these response electrical signals are related/dependent on the stimulation signal, irregular response signal implies that the intensity or mode of the initial stimulation electrical pulse was not optimized (for example, a particular set of electrodes were inadvertently moved to a slightly different position or distance away from the nerve cell location. Therefore, if the ECAP response signals can be measured with sufficient resolution, they can be well utilized to re-set the applied pulse signals for subsequent optimized (or corrected) electrical stimulation processes.

These, and other, features and aspects are described in greater detail in the drawings, the description and the claims.

### BRIEF DESCRIPTION OF THE DRAWINGS

The features and advantages of the present invention will become more apparent by describing in detail exemplary embodiments thereof with reference to the attached drawings listed below:

FIG. 1. Metallic nanopillar growth by hydrothermal growth of seed oxide (such as Co-oxide, Fe-oxide, alloy oxide nanopillars) first, followed by sputter coating with biocompatible Pt, Pt—Ir or MP35N alloy, then reducing the core oxide into metal by high temperature, hydrogen reduction heat treatment (in H₂, H₂-containing atmosphere of forming gas, ammonia, etc).

FIG. 2. (a) Anodizing process to create vertical nanopores on substrate surface, (b) Created anodized Al₂O₃ membrane with hole array through which guided nanopillar growth on neuro-stimulation electrode alloy wire (or plate) surface is performed, (c) Biocompatible Pt, Pt—Au, Pt—Ir, Pt—Au—Ir, other noble metal/alloys, or MP35N alloy electrode nanopillars radially or vertically grown through the nanopores. Example desired composition range of Pt—Ir alloy is 5-30% Ir, preferably 10-20% Ir. Alternatively, pure Pt nanowires can be grown, with Ir film sputter coated, followed by annealing to diffuse Ir into the Pt matrix, to at least form Pt—Ir alloy skin surface, or Ir oxide skin surface can be produced.

FIG. 3. Vertical metal alloy nanopillar array by e-beam lithography or nano-imprint lithography patterning and metal deposition by electrodeposition or sputter deposition followed by lift-off processing.

FIG. 4. (a) Schematic illustration of RF plasma processing of MP35N, Pt—Ir or other neural stimulation electrode alloy wires. (b) SEM micrographs of RF processed nanowires on MP35N (Co—Ni—Cr—Mo alloy) alloy surface depicting high-aspect-ratio vertical aligned structure, (c) Pt-10% Ir alloy surface with RF processed nanowire array. The RF power is typically 100-200 Watt and the process time is about 5-10 minutes.

FIG. 5. Impedance of Pt—Ir and MP35N electrodes vs operating frequency, with vs without RF plasma processing to increase surface area. Significant impedance decreases occur in the lower frequency range (<1000 Hz) and both Pt—Ir & MP35N exhibit an approximate 50% decrease with one round of surface texturing. MP35N electrode surface processed five times (5×) shows an order of magnitude decrease in impedance. A similar behavior is anticipated with Pt-10% Ir alloy. (Dulbecco's Phosphate-Buffered Saline (PBS) used as the electrolyte.) Such a reduced electrical impedance (less resistive loss of electricity at bio interfaces) allows the use of less electricity and a much longer time use of battery power for neural stimulation in the case of implanted battery pack arrangement. For example, if the impedance is decreased by a factor of 10, the battery power use could be reduced by as much as a factor of 10.

FIG. 6. Nanopillar growth directions on neural stimulator electrodes. (a) Tilted nanopillars near the corners or edges of the electrode as the electrical field in RF plasma etching tends to be perpendicular to the local surface regions, which is undesirable as they cause unwanted electrical signals sent to wrong directions or at wrong angles. (b) If a nanopatterning approach is utilized, a very uniform nanopillar array is obtained without the formation of such undesirable tilted nanopillars on unwanted locations.

FIG. 6. Nanopatterning to form more uniform, periodic (or non-periodic) nanopillars or nanowires protruding from the substrate electrode alloy for decreased electrical signal impedance and also for reduced consumption of electrical/battery energy. Optionally Au-coated or Ti/Au coated on the nanopillar surface for corrosion resistance and anti-biofouling.

FIG. 7. SEM micrograph showing excellent nanopillar formation by ICP plasma etch of MP35N alloy wire (250 um diameter). The ICP gas used was 25% Cl in Argon at 30 sccm flow rate, with the plasma chamber pressure of 10⁻² torr, at 200 watt power for 10 minute. The nanopillar type structure radially grown on the alloy wire surface has about 1˜2 um length and a high aspect ratio of ˜5-10.

FIG. 8. Use of high pressure Ar atmosphere for deeper penetration of sputter deposited electrode alloy into deeper cavity to form high aspect ratio, periodic (or non-periodic) nanopillars or nanowires. Such nanostructures protruding from the substrate electrode alloy enables decreased electrical signal impedance and also for reduced consumption of electrical/battery energy. Optionally Au-coating, Ti/Au coating or other noble metal coating may be added on the nanopillar surface for corrosion resistance and anti-biofouling.

FIG. 9. Use of SiO₂ type, island disk array mask via nano-patterning by e-beam, ion-beam, nano-imprint lithography, (NIL), EUV lithography, etc. and deposition. The disk-shape masks are utilized like a shadow mask to perform ME (reactive ion etch) or chemically etch the electrode to form nanopillars into the electrode alloy base. Such a high density array of elongated nanopillars reduces the overall electrical impedance within a biological solution (or in an in-vivo environment like implanted neural-stimulation or neural-monitoring electrodes inside a human body). Such a reduced electrical resistive loss of energy enables the implanted power source such as batteries to last much longer. Optionally the nanopillar or nanowire surface can be coated with Au (or with an added refractory-metal-base adhesion layer like Ti film) by e.g., sputter deposit, evaporation deposit, etc) for improved corrosion resistance and enhanced anti-biofouling.

FIG. 10. Electrodeposition of elongated electrode nanopillars into patterned holes in the resist mask. Dissolving away of the polymer or ceramic resist results in an electrode surface with desirable, protruding, nanopillar array.

FIG. 11. Utilization of pre-made patterned seed for longer nanopillar formation by RF plasma etching.

FIG. 12. Seed nanopillar type structure in one electrode alloy which is template-transferred to another electrode alloy underneath during continued etch process (e.g., plasma etch or chemical etch).

FIG. 13. Nichrome (Ni-20% Cr) alloy sacrificial seed layer pre-deposited (2 um thick) and RF plasma textured (175 Watt/15 min/5 cycles) to transfer the nanopillar structure that occur first on Nichrome layer into the Pt—Ir alloy base underneath. The Pt—Ir wire was 250 um diameter×10 cm long. Experimental condition: 5 cycle RF plasma textured at 175 Watt power for 15 min in 30 sccm Ar flow, at 10⁻² pressure. Impedance measured in 1× PBS solution (electrolyte). The impedance at 5 Hz for the given wire sample dimension for bare Pt—Ir wire was ˜210 ohm, impedance for the RF textured Pt—Ir was ˜200-250 ohm, and the impedance for the Nichrome coated Pt—Ir after nanotexturing was ˜180 ohm. In stimulation mode, AC voltage was applied to the alloy electrode itself. In sensing mode, the AC voltage was applied to the Pt counter-electrode.

FIG. 14. Well textured MP35N alloy with reduced impedance can be further improved by surface coating with higher conductivity metal or alloy (such as Pt, Pt—Ir, Au, or alloys of noble metals). Such addition of Pt, Pt—Ir, Au or alloy of noble metal can be accomplished by physical vapor deposition (e.g., sputtering or evaporation) or by chemical processing (e.g., electroless deposition) or electrochemical deposition from aqueous solution containing Pt or Pt/Ir ions.

FIG. 15. Pt coating effect of substantially lowering the impedance of optimally surface textured MP35N wire (by RF plasma), 250 um diameter and ˜10 cm long. Experimental condition: 5 cycle RF plasma textured at 175 Watt power for 15 min in 30 sccm Ar flow, at 10⁻² pressure. Impedance measured in 1× PBS solution (electrolyte). The impedance at 5 Hz for the given wire sample dimension for MP35N was ˜620 ohm, which was reduced to ˜260 ohm by 5 cycles of this particular RF texturing. Thin Pt film coating on the textured MP35N additionally lowers the impedance to ˜120 ohm for both stimulation mode and sensing mode.

FIG. 16A. Experimentally measured sensing signal by electrode wires when a pulse signal train of 750 mV amplitude at 1 KHz frequency with 1 usec pulse width is applied to the Pt counter electrode in a 0.1× PBS solution.

FIG. 16B. A thick ground beef solution (73% solid) is used for similar experimental measurements of sensing signal by electrode wires when a pulse signal train of 750 mV amplitude at 1 KHz frequency with 1 usec pulse width is applied to the Pt counter electrode.

FIG. 17. Length increase of nanopillars by electrodeposition onto pre-made nanopillar seed electrode, e.g., from 500 nm to 2 um. Existing nanopillar tips serve as nucleating sites for electrodeposition. Such increased aspect ratio of nanopillars reduces the electrode impedance for easier application of SCS signals.

FIG. 18. Electrode lead and electrode extension with subdivided structure having more advantageous response of reduced eddy current, reduced heating and battery energy savings on higher frequency electrical stimulation. Optional annealing heat treatment can be utilized for intermediate softening or better bonding between adjacent subdivided wires.

FIG. 19. Ultra-fine-grained electrode lead and electrode extension with subdivided structure having more advantageous response of reduced eddy current, reduced heating and battery energy savings on higher frequency electrical stimulation.

FIG. 20. Alteration of electrode nanopillar structure or composition to reduce the eddy current loss and to allow higher frequency electrical signal pulsing if needed. (a) nanopillar array structured electrode, (b) further sub-divided nanopillar dimension for higher frequency operation, (c) microstructural sub-division with finer grain size or addition of second phase particles (e.g., by co-deposition of inert oxide like Al₂O₃, refractory oxide like ZrO₂, more stable rare earth oxide like CeO₂, during deposition of alloy into the nanopore array) or bleeding of oxygen or air for intentional oxidation or oxide particle formation. The presence of particles in the alloy or grain boundary will increase the electrical resistivity for reduced eddy current loss for easier operation of electrical pulses at a higher frequency regime.

FIG. 21. Modification of surface of electrode nanopillars by coating with nano-grained thin film (e.g., by sputtering of the same or different electrode alloy, such as Pt, Pt—Ir, MP35N alloy). The resultant nano-grain structure (less than 50 nm, preferably less than 20 nm average diameter) has a higher electrical resistivity, which reduces the eddy current loss and allows higher frequency electrical signal pulsing if needed. (a) Periodic nanopillar array structured electrode, (b) Surface coating with nano-grained layer on periodic nanopillar surface, (c) Surface coating on non-periodic nanopillar array.

FIG. 22. Nanopillar shape alteration for enhanced electrical pulse focusing and improved directing to reduce waste of electrical energy, using either tip-sharpened geometry or partial shielding of lower portion of nanopillars with insulating barrier material.

FIG. 23. Sharp cone geometry carbon electrode array made by electron-beam patterning, nanoimprint patterning, or photolithographic patterning of catalyst (such as Ni) during CVD plasma growth of carbon nanotube (nanocone) array. The surface of the carbon nanocone array can optionally be coated/protected with a coating of Pt, Pt—Ir, MP35N or other neural stimulation electrode material, e.g., by sputtering, evaporation, electrodeposition or electroless deposition.

FIG. 24. Selective-position antibiofouling coating of MP35N or Pt—Ir vertical nanopillar array to maintain the electrical conductivity while providing the antibiofouling state. (a) Vertical (or radial) alloy nanopillar array by nanopatterning (b) Selective height masking by PMMA mask layer deposit, (c) Deposit antibiofouling coating only to the tip region of the protruding nanopillars such as by using PTFE (sputter deposit), PEG (dip coating or spin coating), (d) lift-off process to remove the PMMA mask and a create surface antibiofouling structure yet electrically highly conductive due to the still large-surface-area, exposed nanowire regions underneath.

FIG. 25. Example spinal neural stimulation electrode array, e.g., for pain management. (a) Vertebrae column, (b) Neural stimulating electrode (e.g., SCS (spinal cord stimulator)), (c) A laminotomy is made in the bony vertebra to allow room to place the leads. The leads are positioned in the epidural space above the spinal cord to deliver electrical current to the area of pain.

FIG. 26. Spinal cord stimulator (SCS) device form-factor effect, (a) Regular SCS stimulator package with a large, bulky battery shape and pulse generator implanted near the hip region, with the “Lead” having attached electrodes positioned in the epidural space, (b) a convenient, smaller battery can be employed as enabled by using electrodes having reduced impedance and reduced need for battery power, (c) further size reduced, rod-shape battery can be a part of the lead wires (extension wires) for more compact implant geometry (multiple rod batteries can be connected in-series for higher voltage or in-parallel for higher current), taking a much smaller space.

FIG. 27. Spinal cord stimulator (SCS) device form-factor effect, (a) Regular SCS stimulator package with a large, bulky battery shape and pulse generator implanted near the hip region, with the “Lead” having attached electrodes positioned in the epidural space, (b) a convenient, smaller battery can be employed as enabled by using electrodes having reduced impedance and reduced need for battery power, (c) with a smaller battery capacity becoming sufficient, a single-incision surgery can be made feasible, instead of two incisions for lead wire placing and battery placement. This provides a patient-centric advantage.

FIG. 28. Feedback controlled neural stimulation. (a) Epidural space near spinal cord for electrode implanting, (b) ECAP-controlled or other response-signal-controlled adjustment of pulse stimulation with altered/optimized pulse intensity, mode and frequency.

FIG. 29. Improved sensing electrode comprising nanopillar textured MP35N alloy wire electrode. The sense signal amplitude matches the sensing current level of 5 regular, non-textured electrodes of the same alloy wire, thus far exceeding the sensitivity of 1 regular electrode. The nanopillar textured electrode is also the only one to resolve the downward pulse applied for the testing.

FIG. 30. On-chip (or on Si) nanopillar tip-sharpening for enhanced electric field concentration and improved focusing of electrical pulsing.

FIG. 31. ECAP feed-back controlled neurostimulator system with control microprocessor chips positioned on the lead itself, with the chip powered either by the implanted battery pack (enabled to be small enough due to our low impedance electrodes), with control software optionally embedded in the chip. The neuro-stimulator electrode array (some of which can also serve as ECAP neural response signal sensors) can be prepared in various inventive ways, including nanofabrication on Si or other semiconductor substrates, which also allows an easier integration of microprocessor or other signal process chips placed directly on the lead itself.

FIG. 32. One example method of protecting the surface nanopillars from mechanical damage on insertion surgery into epidural space in the spinal cord, by utilizing recessed geometry or temporarily protective coating.

FIG. 33. Example manufacturing procedure for large-scale industrial production of neuro-stimulation electrode array fabrication using chemical, electrochemical or electrophoretic approaches.

FIG. 34. Schematic illustration of Inductively Coupled Plasma (ICP), RF or microwave plasma processing of electrode alloys (such as MP35N or Pt—Ir alloy wires/ribbons) in a continuous or semi-continuous manner for industrial manufacturing.

FIG. 35. Use of airlock system for ease of supply of materials to be plasma etch nanotextured by ICP, RF plasma or microwave plasma processing of electrode alloys (such as MP35N or Pt—Ir alloy wires/ribbons) in a continuous or semi-continuous manner for industrial manufacturing.

FIG. 36. Electrode array configurations can be of geometrical shape, (a) ring array type, (b) paddle type. Each electrode can be utilized as a stimulating electrode for specific location of human body, and can also serve a dual function of pulsing electrode and sensing (e.g., for ECAP signals) electrode. Alternatively, the pulsing and sensing electrode can be separately provided if desired.

FIG. 37. Assembly into a neural stimulator lead (e.g., spinal cord stimulator lead) using an array of low impedance, ring-shape electrodes, e.g., comprising nanopillared and/or IrO₂-coated, structure.

FIG. 38. Mechanically compliant (springy), electrode-gap-reducing electrode structure. Onto the base electrode surface, mechanically flexible, micro-spring-like extension microwires are added (e.g., on the ring electrode surface or rectangle electrode surface on a paddle lead). The springy microwires can be temporarily retained in a compressed state, which can later be released when the water dissolvable retainer material (e.g., sucrose, honey, gelatin or other water-soluble solid) is dissolved away inside the human body after implanting of the neural stimulator device at the desired location.

FIG. 39. Protective shoulder to mechanically shield the nanopillar type, impedance-lowering structures during lead insertion operation as well as during assembly, handling, shipping, etc. The protective shoulder can be fabricated by machining, etching, metal press-forming, or by additive manufacturing. The shoulder can be made of the same ring material or other material.

FIG. 40. Manufacturing of ring electrodes (closed ring or split ring) having low impedance surface can be achieved by e.g., (i) plasma surface texturing to form nanopillar surface structure, (ii) chemical etching, (iii) anodization, (iv) electrochemical deposition of radial nanopillars, etc. Such processing can be performed with (a) a long cylinder first which is then sliced into short width ring electrodes, (b) processing or a stacked short rings followed by separation, or (c) processing of flat strips followed by bending/curbing into a ring configuration.

FIG. 41. Drug impregnated in the dense nanopillar forest of Pt—Ir or other electrode alloys (e.g., antibiotics, steroids, neuromodulator drugs, small molecule drugs, etc), to be slowly released after the neurostimulator device such as pain-reducing spinal cord stimulator is implanted and the temporary cap (water dissolvable material) is dissolved away at the implant site. The drug release speed can be controlled by the forest density, viscosity/concentration/solubility of the liquid drug, the nature, thickness, porosity of the temporary cap.

FIG. 42. Example impedance measurement setup with a basically saline type PBS solution vs pseudo-physiological environment of freshly ground steak solution as a tissue analog. The electrode was MP35N alloy wire, standard smooth-surface wire electrode vs five times RF plasma treated at 900° C. to further elongate the nanopillar array.

FIG. 43. Example impedance measurement set up for PBS vs ground steak solution.

FIG. 44A. Impedance measurement data in (a) PBS solution vs (b) in tissue analogue (pseudo-physiological environment).

FIG. 44B. Comparative impedance reduction behavior of PBS vs pseudo-physiological meat solution for MP35N nanopillar electrode relative to the regular electrode.

FIG. 45. Chemical or electrochemical pre-etch treatment to produce initial surface cavities to make the subsequent nanopillar formation easier during plasma etch process. Either inorganic or organic acids, electrolytes, can be utilized.

FIG. 46. Island array mask via high melting point metal/alloy island deposition using sputtering, electrodeposition, etc, optionally using nanotemplates such as anodized aluminum oxide (AAO) membranes or block copolymer (BCP) membranes.

FIG. 47. Pre-treatment modification of previously plasma textured electrode surface by mechanical, chemical, electrochemical, reactive ion removal of existing nanopillar type structures, followed by second plasma etch texturing for higher density, taller and more uniform nanopillar structures.

FIG. 48. Pre-deposit a nano membrane/mask to allow a subtractive process of making selective local surface pitting through the open regions of the membrane/mask.

FIG. 49. Pre-deposit a nano membrane/mask to produce selective local surface nano-protrusions to serve as guiding feature or nuclei feature for subsequent plasma etch texturing. The protrusion can be made by sputter deposition, evaporation, CVD, electrodeposit, etc) of either an identical material as the electrode (e.g., Pt—Ir alloy), or a different material (e.g., high mp metal/alloy or ceramic material protruding mask).

FIG. 50. Use of plastic and elastic deformation of nanopillars and associated nanogeometry by drawing the electrode wire through a die, rolling deformation of a strip of electrode paddle, contact sliding, contact rotating deformation, etc to bend nanopillar type structures, so as to expose previously hidden substrate regions (by nanopillar forest) for additional plasma etch. The strained nanopillar surfaces, because of plastic and elastic deformation, have more defects, which are also more favorable places for initiation of plasma etching. Such a higher density nanopillar type structures will contribute to lowered impedance and increased sensing signal (e.g., ECAP type signals).

FIG. 51. Some portion of the electrode surface area needs to be free of nanopillar or other nanostructures (e.g., on ring electrode cross-sectional surfaces and ring-inside-surfaces), so as to prevent inadvertent falling off of loose metallic nanopillars, or to avoid intereference with spot welding with extension conductor wires. (a) These ring cross-sectional surfaces and ring-inside-surfaces can be blocked from the plasma by an insulating or high melting point layer metal or ceramic coating (temporary or permanent) such as biocompatible TiO₂, Ta₂O₅, other refractory oxides, CrO₂, Al₂O₃, MgO, etc) during plasma etch texturing, so as to prevent nanopillar formation. (b) Another approach to prevent nanopillar formation is to assemble a stack of electrode rings together so that the cross-sectional regions and inside the ring regions are protected from plasma etch texturing.

FIG. 52. Location-controlled enhancement of plasma etch texturing. (a) Nanopillar/nanostructure forest on electrode alloy surface by plasma etch texturing using active gas or inert gas, (b) Nanopillar/nanostructure top is masked by higher mp or lower-rate-plasma-etchable metal or ceramic cap (using e.g., oblique incident sputtering or tip coating by dipping or particle solution spraying). The masking of nanopillar top surface helps to prevent the nanopillar height from getting continuously and excessively eroded during plasma etch, (c) Nanopillar/nanostructure top and side protected by sputtered lower mp or less-plasma-etchable coating so that the plasma etching more selectively continues at/into the valley locations to make the nanopillars taller, (d) improved, taller nanopillar/nanostructure configuration for lowered impedance and higher signal sensing capability.

FIG. 53. Electrode surface coating with chemically and mechanically stable nanoparticle structures for surface area increase, or surface porosity increase by selective etching (chemical or RIE etching) for reduced impedance.

FIG. 54. Hierarchical electrode surface modification by deposition of porous material (same as the electrode base or different biocompatible material), such as a nanoporous Pt or Pt—Ir layer on the surface of previously formed nanopillar type protruding features on Pt or Pt—Ir electrode (or other spinal cord stimulation or deep brain stimulation type electrodes).

FIG. 55. Surface modification of neural stimulation electrodes by deposition of porous material for reduced impedance. The porous deposit can be the same material as the electrode base or different biocompatible material, such as porous Pt or Pt—Ir on Pt or Pt—Ir electrode surface (or other spinal cord stimulation or deep brain stimulation type electrodes). (a) Porous material added on the surface of ring shape electrodes, (b) Porous layer added on paddle type electrode surface.

It is understood that the above-described embodiments are illustrative of only a few of the many possible specific embodiments which can represent applications of the invention. Numerous and varied other arrangements can be made by those skilled in the art without departing from the spirit and scope of the invention.

### DETAILED DESCRIPTION OF THE INVENTION

The impedance can be reduced by various methods, which are also described in this invention. Nanostructures provide greatly increased surface area which affect biological, mechanical, chemical and electrical behavior. The large surface area in nanostructured electrodes, often at least by a factor of two, preferably at least by a factor of five increased as compared with typical bulk macro electrodes, can provide unique properties and advantages in functional electrical stimulations such as spinal cord stimulations (SCS) and deep brain stimulations. While surface roughness can be introduced on electrode surfaces such as metallic electrodes made of Pt, Pt-10% Ir, or MP35N alloy (35% Co-35% Ni-20% Cr-10% Mo in wt. %) by a number of different methods, e.g., by sintering of powered starting materials to obtain porous surface, chemical or electrical etching or plasma etching, these methods usually produce random nanostructures. Innovative approaches are employed in this invention to prepare desirably large-surface-area electrodes, such as comprising nanoporous or preferably nanopillared structures, advantageous means/structures for imparting lowered electrode electrical impedance, for increasing sensing signals for detection of neural activities, for enabling feedback-based neural stimulation therapies, for providing anti-biofouling properties, for devising methods of providing mechanical flexibility and high amplitude electrical pulses, for mechanically protecting the nanopillar type structures with various configurations, as well as various other unique embodiments as described in more detail below.

[A]. Foreign Material Nanopillar Template Addition to Electrode Surface, Followed by Optional Biocompatible Thin Film Coating and/or Reduction to Metallic Base

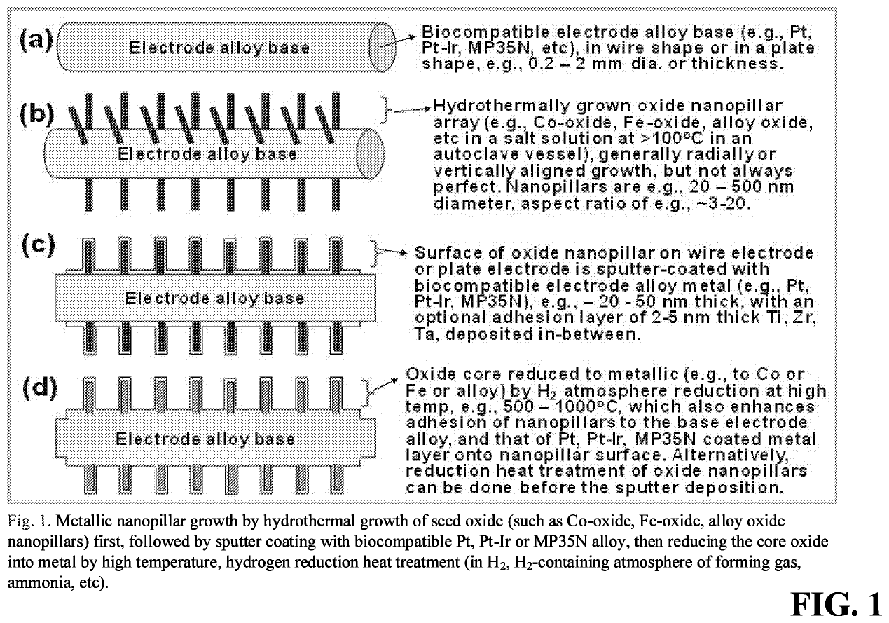

Referring to the drawings, FIG. 1 schematically illustrates an example process of metallic nanopillar growth, according to the invention, by utilizing hydrothermal growth of seed oxide (such as Co-oxide, Fe-oxide, Ni-oxide, Ti-oxide, refractive metal oxide, alloy oxide, in the form of nanopillars, nanowires, nanoribbons or other protruding nanostructures) first as an interim process, followed by sputter coating with biocompatible Pt, Pt—Ir or MP35N alloy, then reducing the core oxide into metallic nanopillar by high temperature, hydrogen reduction heat treatment. For reduction of oxide to metal, an annealing heat treatment can be utilized at 300° C. to 1,000° C., with the heat treatment atmosphere selected to be H₂ gas or hydrogen-containing atmosphere such as a forming gas (5-10% H₂ gas mixed with a nitrogen or argon gas base). Other gases containing hydrogen can also be utilized, such as an ammonia (NH₃) gas type atmosphere.

To produce nanostructures by hydrothermal process, biocompatible electrode alloy base (e.g., Pt, Pt—Ir, MP35N, and so forth) in wire shape, ribbon shape or in plate shape, e.g., 0.2-2 mm diameter or thickness, can be placed in an autoclave vessel to grow oxide nanopillar array (e.g., Co-oxide, Ni-oxide, Ti-oxide, refractive metal oxide, alloy oxide, in the form of nanopillars, nanowires, nanoribbons or other protruding nanostructures) in a salt solution at >100° C.). For wire shape substrate, generally radially grown nanopillars or related nanostructures are obtained while for plate shape substrate, vertically aligned nanopillars or other nanostructures are grown. Desired nanopillars are e.g., 20-1,000 nm in average diameter (preferably 50-200 nm), having an aspect ratio of e.g., ˜3-50, preferably 5-20.

Once the oxide nanopillars are grown by hydrothermal process, the surface of oxide nanopillar are sputter-coated with biocompatible electrode alloy metal (e.g., Pt, Pt—Ir, MP35N), e.g., −20-50 nm thick, with an optional adhesion layer of 2-5 nm thick Ti, Zr, Ta, deposited in-between. This is followed by a reduction treatment to reduce and convert the oxide core to metallic material (e.g., to Co or Fe or alloy) by H₂ atmosphere reduction at high temp, e.g., 500-1000° C. for 10 min to 24 hrs, which also enhances adhesion of nanopillars to the base electrode alloy, and that of Pt, Pt—Ir, MP35N coated metal layer onto nanopillar surface. The sequence of processing can optionally be changed, e.g., the reduction heat treatment of oxide nanopillars to metallic nanopillars can be done first before the sputter deposition.

At FIG. 1, illustrated is a metallic nanopillar growth by hydrothermal growth of seed oxide (such as Co-oxide, Fe-oxide, alloy oxide nanopillars) first, followed by sputter coating with biocompatible Pt, Pt—Ir or MP35N alloy, then reducing the core oxide into metal by high temperature, hydrogen reduction heat treatment (in H₂, H₂-containing atmosphere of forming gas, ammonia, etc). Furthermore, illustrated at reference letter (a) is a biocompatible electrode alloy base (e.g., Pt, Pt—Ir, MP35N, etc.), in wire shape or in a plate shape, e.g., 0.2-2 mm diameter or thickness. Illustrated at reference letter (b) is a hydrothermally grown oxide nanopillar array (e.g., Co-oxide, Fe-oxide, alloy oxide, etc. in a salt solution at >100° C. in an autoclave vessel), generally radially or vertically aligned growth, but not always perfect. Nanopillars are e.g., 20-500 nm diameter having an aspect ratio of e.g., ˜3-20. At reference letter (c), illustrated is a surface of oxide nanopillar on wire electrode or plate electrode is sputter-coated with biocompatible electrode alloy metal (e.g., Pt, Pt—Ir, MP35N), e.g., −20-50 nm thick, with an optional adhesion layer of 2-5 nm thick Ti, Zr, Ta, deposited in-between. At reference letter (d), illustrated is an oxide core reduced to metallic (e.g., to Co or Fe or alloy) by H₂ atmosphere reduction at high temp, e.g., 500-1000° C., which also enhances adhesion of nanopillars to the base electrode alloy, and that of Pt, Pt—Ir, MP35N coated metal layer onto nanopillar surface. Alternatively, reduction heat treatment of oxide nanopillars can be done before the sputter deposition.

### [B]. Patterned Additive Electro-Deposition or Physical Vapor Deposition of Nanopillared, Biocompatible Metal Electrode Material

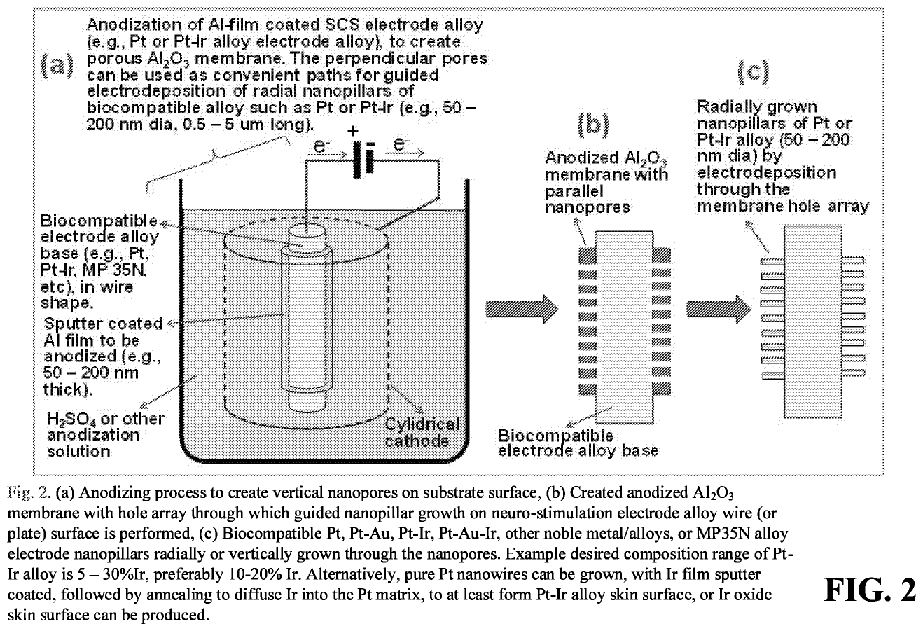

(1). Use of nanoporous template such as aluminum oxide membrane to deposit elongated metal array. Instead of hydrothermal process of FIG. 1, an alternative process of forming an aligned nanopillars on electrode surface is to utilize an anodized Al₂O₃ membrane for guided nanopillar growth, as illustrated in FIG. 2. This can be performed on SCS electrode alloy wire (or plate) surface, such as biocompatible Pt, Pt—Ir or MP35N alloy electrode, by first depositing an Al film (e.g., 50-200 nm thick, by sputtering or evaporation) on the electrode alloy substrate surface to be anodized, and then performing electroplating through the pores.

Electrochemical anodization of Al-film coated SCS electrode alloy (e.g., Pt or Pt—Ir alloy electrode alloy) can create porous Al₂O₃ membrane. The perpendicular pores can be used as convenient paths for guided electrodeposition of radial nanopillars of biocompatible alloy such as Pt or Pt—Ir (e.g., 50-200 nm dia, 0.5-5 um long). The anodization is performed in H₂SO₄ or other anodization solution, while a voltage is applied between the anode and the cathode, e.g., 10-120 Volts. The electrochemical anodization etching produces A1203 membrane with near-parallel elongated nanopores, vertically to the flat substrate and radially in the case of round substrate. Radially positioned nanopillars of Pt or Pt—Ir alloy (50-200 nm dia) can be grown by electrodeposition through these near-parallel membrane hole array. Thus, anodized Al₂O₃ membrane in the form of thin concentric cylinder on a SCS type wire or rod geometry electrode can be utilized to perform a follow-up deposition of metallic alloy (such as Pt or Pt—Ir) guided along the elongated paths, thus producing radially positioned nanopillar array of Pt or Pt—Ir (e.g., 50-200 nm diameter, 0.5-20 um tall) attached onto the base rod or wire electrode of Pt or Pt—Ir, e.g., 1-2 mm diameter. The desired composition range of Pt—Ir alloy is 5-30% Ir, preferably 10-20% Ir.

The Pt—Ir nanopillars or other electrode alloy nanopillars on Pt—Ir base rod, wire or ring can optionally annealed at 300-900° C. for the purpose of further increasing the adhesion/bonding of the Pt—Ir alloy nanopillar to the base Pt—Ir alloy substrate.

Turning now to FIG. 2, illustrated at (a) is an Anodizing process to create vertical nanopores on substrate surface, illustrated at (b) is a created anodized Al₂O₃ membrane with hole array through which guided nanopillar growth on neuro-stimulation electrode alloy wire (or plate) surface is performed, and illustrated at (c) is a biocompatible Pt, Pt—Au, Pt—Ir, Pt—Au—Ir, other noble metal/alloys, or MP35N alloy electrode nanopillars radially or vertically grown through the nanopores. Example desired composition range of Pt—Ir alloy is 5-30% Ir, preferably 10-20% Ir. Alternatively, pure Pt nanowires can be grown, with Ir film sputter coated, followed by annealing to diffuse Ir into the Pt matrix, to at least form Pt—Ir alloy skin surface, or Ir oxide skin surface can be produced.

(2). Use of nanoporous block copolymer membrane template to deposit elongated metal array. Instead of AAO type processing, an alternative method is to utilize diblock or triblock copolymer type polymer coated on the electrode alloy lead wire surface (such as Pt, Pt—Ir, Pt—Au—Ir or MP35N) to produce a desirably dimensioned vertical or radial nanohole array. Such diblock or triblock copolymers (e.g., Poly(styrene-block-methyl methacrylate), also called PS-PMMA, or polystyrene-block-poly (4-vinylpyridine), also called PS-b-P4VP, which on two-phase decomposition of the polymer, produces vertical or radial nanohole array through which the biocompatible metallic nanopillars such as Pt or Pt alloy can be electrodeposited, electroless deposited or sputter deposited, similarly as illustrated in FIG. 2. The polymer matrix can then be dissolved by solvent or burned away so that the deposited nanopillar type nanostructure is exposed for the purpose of increasing the surface area and reducing the impedance for improved neural stimulation or neural sensing.

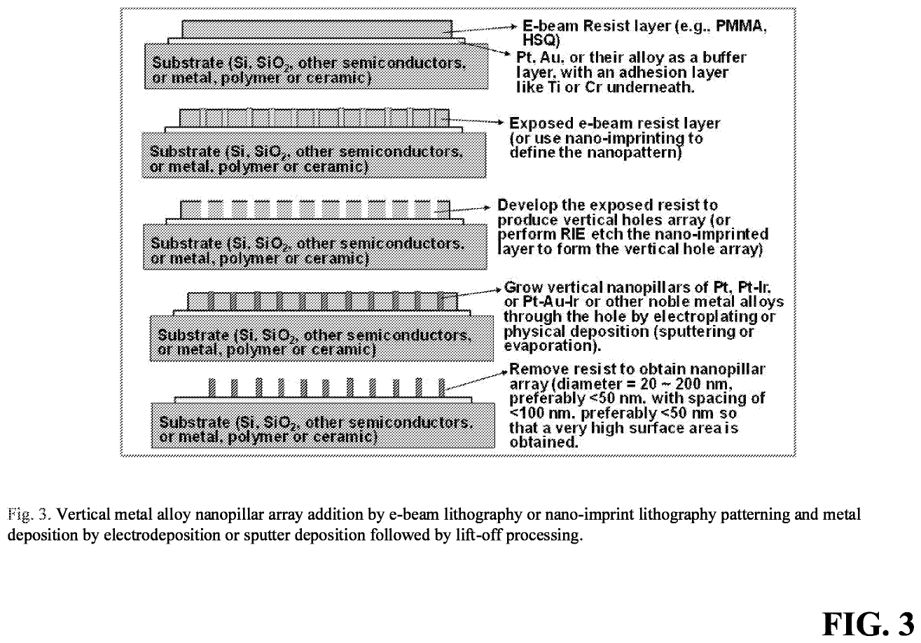

(3). Lithographically patterned membrane template to deposit elongated metal array—Yet another alternative approach is to utilize polymer nanopatterning, for example, e-beam lithography or nanoimprint lithography. Extremely fine nanopillars with diameter as small as ˜20 nm and aspect ratio as high as 10 can be obtained as shown in FIG. 3. Such nanopillars (e.g., Pt or Pt—Ir or Pt—Au—Ir) can be made taller, if desired, by employing electrodeposition, which will add more alloy material to the tip of elongated nanopillars, thus making the nanopillars to be even taller with an increased aspect ratio.

### [C]. Subtractive Process of Creating Nanopillar and Related Structure Such as by RF Plasma Etching

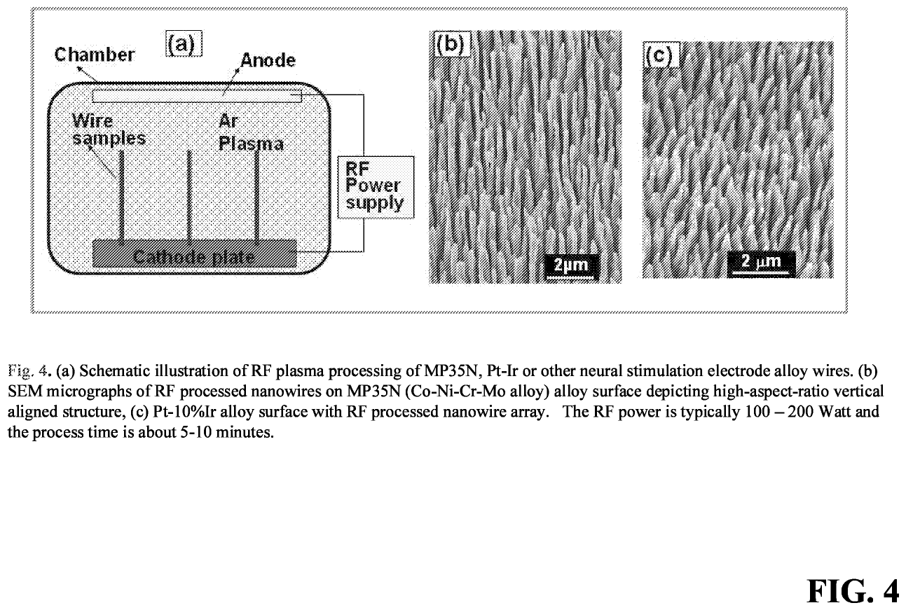

(1). Use of inert gas-based RF plasma texturing. Yet another method for growing nanopillar array is to utilize a RF plasma etching as described in FIG. 4. This high temperature plasma etching process in partial Ar atmosphere is a relatively fast process, typically taking less than e.g., 20 minutes to produce vertically aligned, dense, high-aspect-ratio nanopillar arrays, e.g., arrays with ˜200 nm diameter and ˜1-10 μm height on the surface of metallic ribbon or wire surface. Nanostructures are not necessarily of nanopillar geometry as other nanostructures such as nanoribbons or nanosheets or nanopores, while not as desirable as nanopillars, could also desirably reduce the impedance in electrode performance in neural stimulating or neural sensing. Example alloys that respond to this RF plasma etching include the biocompatible MP35N alloy (35% Co-35% Ni-20% Cr-10% Mo in wt. %), Pt-10˜20% Ir allo, Nichrome alloy (80% Ni-20% Cr), and 316 stainless steel. The process is not an additive process but is rather a substractive process using plasma-based topological etching on the metal surface. The nanopillars are perpendicular to the surface and are therefore radial for the wire sample and vertical for the ribbon sample, as would be anticipated since the electric field tends to be perpendicular to the local surface during plasma processing.

For RF processing, the alloy wires (e.g., 250 μm dia, 10 cm long), ribbons (25 μm thick, 4 cm wide and 6 cm long), cylinders or rings having various dimensions are mounted vertically at the cathode plate base and ˜14 MHz RF plasma (e.g., 1-50 MHz frequency, preferably 5-20 MHz) is provided with an operating condition of base pressure of e.g., 0.02 torr, 30 sccm of Ar gas, RF power of 100-500 Watt, and time of exposure to RF of 5 min to 60 minutes, an example time being about 10 minutes. The sample temperature rises due to RF plasma up to 700-1,000° C. range. The SEM micrographs in FIGS. 4(b) and 4(c) show that the RF processed nanopillars on MP35N and Pt—Ir alloy surfaces which are densely distributed, all vertically aligned on a ribbon surface (or radially aligned on a wire, rod, tube or ring surface). The desired nanopillar dimension is 50-300 nm average diameter and 0.5-30 μm tall. A typically obtained nanopillar dimensions are about 100-200 nm diameter, with the height being ˜1-10 μm tall. Instead of RF plasma, other types of plasma such as DC plasma etching, microwave plasma etching, inductively coupled plasma (ICP) etching, can also be utilized alone or in combination with RF plasma etching.

Illustrated at FIG. 4, reference letter (a) is a schematic illustration of RF plasma processing of MP35N, Pt—Ir or other neural stimulation electrode alloy wires. At reference letter (b) are SEM micrographs of RF processed nanowires on MP35N (Co—Ni—Cr—Mo alloy) alloy surface depicting high-aspect-ratio vertical aligned structure. At reference letter (c) is a Pt-10% Ir alloy surface with RF processed nanowire array. The RF power is typically 100-200 Watt and the process time is about 5-10 minutes.

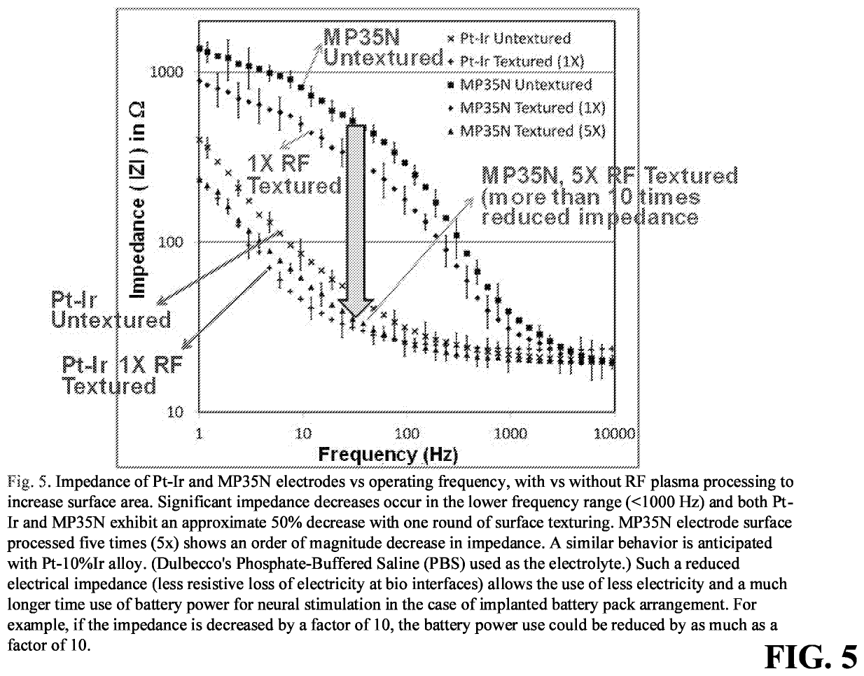

Shown in FIG. 5 is the plot of impedance of Pt—Ir and MP35N electrodes in wire shape as a function of operating frequency, with vs without RF plasma processing to increase surface area. Significant impedance decreases occur in the lower frequency range (<1000 Hz), and both Pt—Ir and MP35N exhibit an approximate 50% decrease with one round of surface texturing. When the MP35N electrode surface is RF plasma processed five times (5×), an order of magnitude decrease in impedance is observed. A similar behavior is anticipated with Pt-10% Ir alloy. Here Dulbecco's Phosphate-Buffered Saline (PBS) solution is used as the electrolyte. A similar results are obtained using a Normal saline solution (0.9% NaCl). Such a reduced electrical impedance (less resistive loss of electricity at bio interfaces) allows the use of less electricity and a much longer time use of battery power for neural stimulation in the case of implanted battery pack arrangement. For example, if the impedance is decreased by a factor of 10, the battery power use could be reduced in principle by as much as by a factor of 10.

Illustrated at FIG. 5. is an impedance of Pt—Ir and MP35N electrodes vs operating frequency, with vs without RF plasma processing to increase surface area. Significant impedance decreases occur in the lower frequency range (<1000 Hz) and both Pt—Ir and MP35N exhibit an approximate 50% decrease with one round of surface texturing. MP35N electrode surface processed five times (5×) shows an order of magnitude decrease in impedance. A similar behavior is anticipated with Pt-10%Ir alloy. (Dulbecco's Phosphate-Buffered Saline (PBS) used as the electrolyte.) Such a reduced electrical impedance (less resistive loss of electricity at bio interfaces) allows the use of less electricity and a much longer time use of battery power for neural stimulation in the case of implanted battery pack arrangement. For example, if the impedance is decreased by a factor of 10, the battery power use could be reduced by as much as a factor of 10.

Such a substantial reduction of impedance is important for neural stimulation and bio-energy harvesting applications, as more current can be supplied/measured for the given voltage, and the required threshold voltage can be reduced for neural stimulation tests/applications.

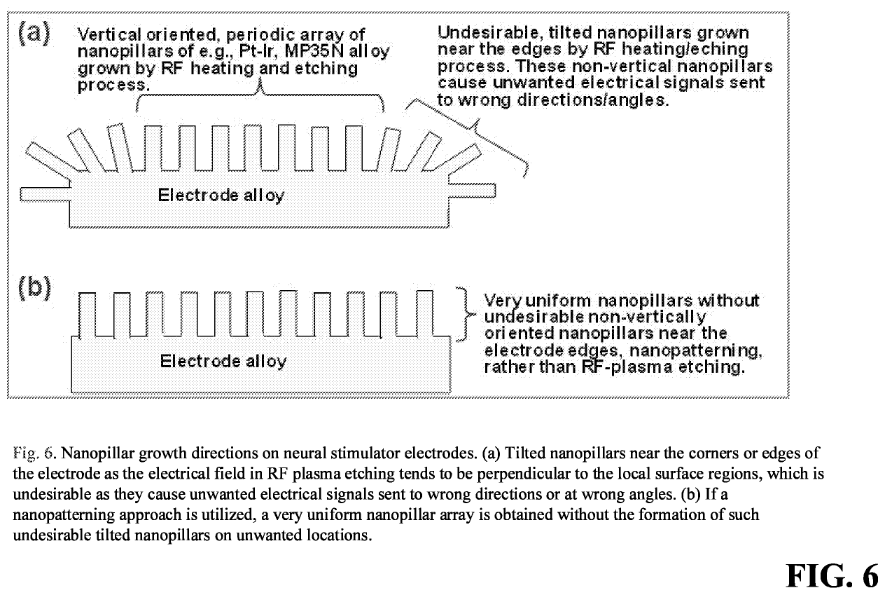

Turning now to FIG. 6, illustrated are nanopillar growth directions on neural stimulator electrodes. At reference letter (a) are tilted nanopillars near the corners or edges of the electrode as the electrical field in RF plasma etching tends to be perpendicular to the local surface regions, which is undesirable as they cause unwanted electrical signals sent to wrong directions or at wrong angles. At reference letter (b), illustrated is a uniform nanopillar array, such that if a nanopatterning approach is utilized, a very uniform nanopillar array is obtained without the formation of such undesirable tilted nanopillars on unwanted locations.

Furthermore, FIG. 6 illustrates nanopillars prepared by RF plasma based high-temperature etching (FIG. 6(a)), while providing a much better electrical stimulation performance than a macroscale bulk electrode surface, there are some disadvantages associated with such RF plasma etched electrodes, which include:

(i) Non-uniform nanowire growth directions near the edges or corners, and hence unwanted electrical signal directions. According to the present invention, this can be mitigated by adding a dummy plate on the edges to prevent electric field bending;

(ii) Large area nano-texturing is generally difficult as RF plasma chamber size (vacuum equipment size) is generally limited. According to the invention, this drawback can be mitigated by utilizing a continuous feeding type plasma etching (using RF, microwave or ICP plasma etching approach), using unrolling and rolling of wound sheets or transporting within the etch chamber a series of pre-cut sheets for continual plasma etch one or several sheets at a time;

(iii) RF plasma etching generally works best with a thin electrode sheet or thin diameter electrode as a thicker electrode is difficult to do surface nano-texturing and nanopillar growth by RF plasma etching due to the thermal conduction loss of heat and temperature drop. However, for applications that do not require thick plate material, such as in the case of neural stimulation electrodes, this is not a major issue; and (iv) High aspect ratio nanopillars are not always easy to obtain on a flat sample.

When a lithography based nanopatterning approach is utilized as illustrated in FIG. 6(b), a very uniform and often periodic nanopillar array is obtained on a flat electrode surface without the formation of such undesirable tilted nanopillars on unwanted locations, and the aforementioned problems and disadvantages are eliminated. For uniformity, reliability and focused electrical signaling, a periodic nanostructure, rather than a random nanostructure, is preferred. If alternative methods of introducing the nanopillars such as in FIG. 1-FIG. 3 are utilized, some of the issues with RF plasma method (such as the nonuniformity aspects, tilted angle nanopillar growth, difficulty of nanopillar growth on large area substrates or thick substrates) can be resolved.

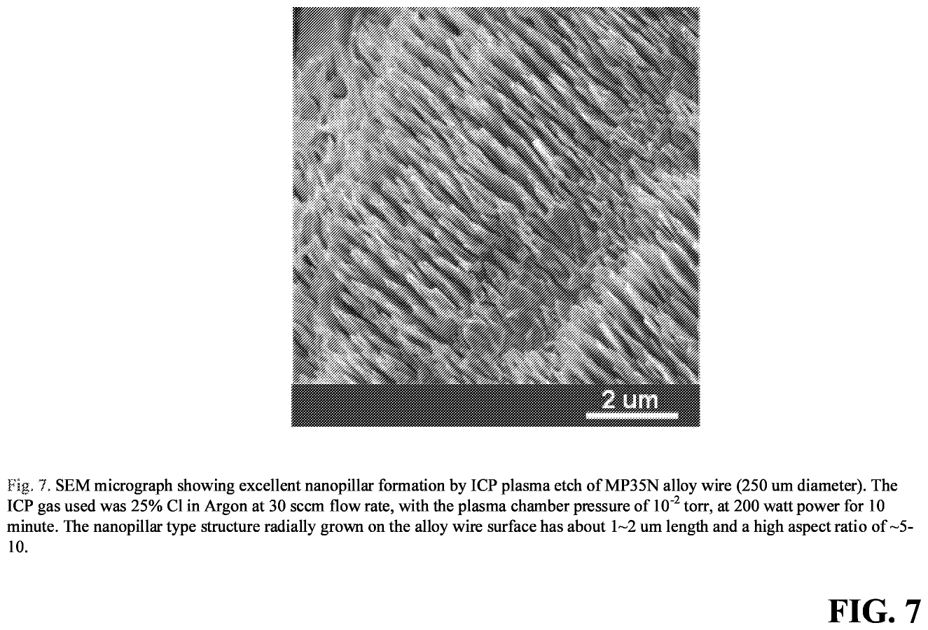

(2). Active gas-containing atmosphere to perform RF plasma texturing or Inductively-Coupled-Plasma (ICP) texturing. The plasma to etch metal electrode surface can be accelerated if a reactive gas such as chlorine- or fluorine-containing Ar gas is used during the plasma etch process. Either RF plasma of ICP plasma can be utilized. ICP (inductively coupled plasma) is a type of plasma source utilizing electric currents as energy source provided by electromagnetic induction in time-varying magnetic fields. An example SEM micrograph of ICP plasma etching using chlorine-containing Ar gas is shown in FIG. 7, which indicates a high-aspect-ratio nanopillar or nanoribbon type structure that can be obtained even with a single cycle.

Furthermore, FIG. 7 illustrates an SEM micrograph showing excellent nanopillar formation by ICP plasma etch of MP35N alloy wire (250 um diameter). The ICP gas used was 25% Cl in Argon at 30 sccm flow rate, with the plasma chamber pressure of 10⁻² torr, at 200 watt power for 10 minute. The nanopillar type structure radially grown on the alloy wire surface has about 1˜2 um length and a high aspect ratio of ˜5-10.

### [D]. Use of High Pressure Ar Atmosphere for Deeper Penetration of Sputter Deposited Electrode Alloy Into a Deeper Cavity.

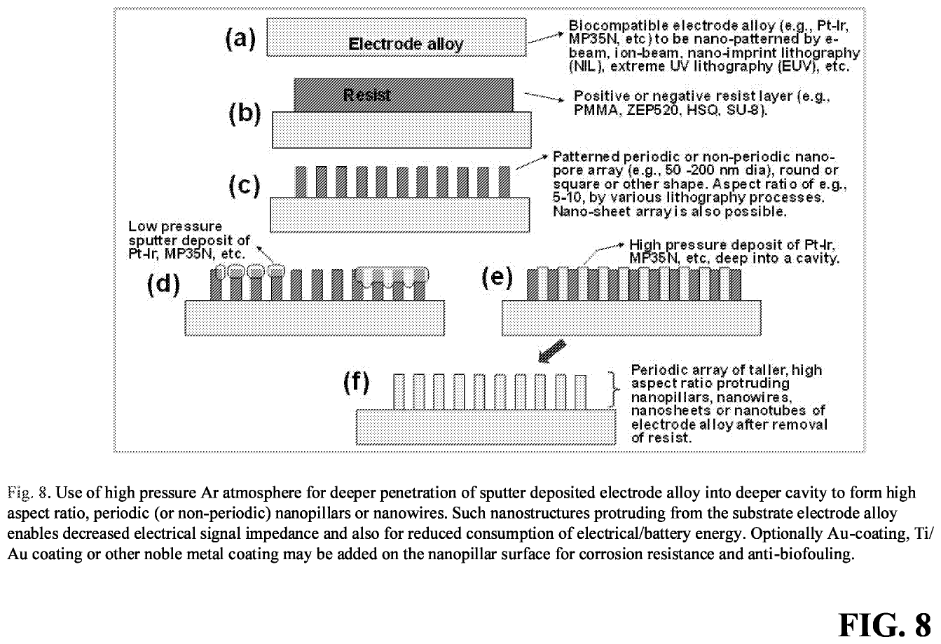

Referring to the drawings, FIG. 8 schematically illustrates a uniform nanostructure of periodic nanopillars, and methods to obtain such structures. The nanopillars are desirably protruding from the substrate electrode alloy with the nanopillars all essentially parallel aligned toward the biological subject (e.g., neural regions to be stimulated), the alignment with the average orientation of the nanopillars deviates from the vertical direction desirably as little as possible, e.g., by at most 45 degree angle, preferably at most 30 degree angle, more preferably at most 15 degree angle. Such subdivided electrode surface provides a very large surface area and also decreases electrical signal impedance in in-vivo environment. The reduced impedance enables less electrical loss of current/voltage energy), which results in desirably reduced consumption of electrical/battery energy.

In FIG. 8, the surface of a biocompatible electrode alloy (e.g., Pt, Pt—Ir, MP35N, etc) is nano-patterned by e-beam lithography, ion-beam lithography, nano-imprint lithography (NIL), extreme UV lithography (EUV), and other lithographical methods. For patterning, the surface of the electrode alloy is coated with a polymer resist layer by spin coating or other means, and then exposed to electron beam, UV optical beam or extreme UV beam by localized pattern writing or a blanket exposure through a mask with desired array of hole pattern. The thickness of the resist layer can be e.g., 10 nm to 500 nm thickness, and the type of resist can be either a positive resist (e.g., PMMA or ZEP520) or a negative resist layer (e.g., hydrogen silsesquioxane (HSQ) or SU-8).

In order to produce a desired, protruding pillar array, the resist is first patterned into periodic nano-pore array (e.g., 50-200 nm dia), round or square or other shape. The aspect ratio of the vertically aligned, periodic holes is in the range of e.g., 2-20, preferably 5-10. (Instead of nanopores, a nano-gap array can also be utilized, to eventually produce a nano-sheet array, rather than a nanopillar array). In order to penetrate into deep cavity and form nanopillar or nanosheet type structures, the invention calls for use of high pressure Ar gas (e.g., higher than 5 mTorr, preferably higher than 10 mTorr, even preferably higher than 30 mTorr) during sputtering, which induces more atomic collision and bouncing off the cavity wall to induce deposition even into the bottom of tall cavities.

Turning now to FIG. 8, illustrated is a use of high pressure Ar atmosphere for deeper penetration of sputter deposited electrode alloy into deeper cavity to form high aspect ratio, periodic (or non-periodic) nanopillars or nanowires. Such nanostructures protruding from the substrate electrode alloy enables decreased electrical signal impedance and also for reduced consumption of electrical/battery energy. Optionally Au-coating, Ti/Au coating or other noble metal coating may be added on the nanopillar surface for corrosion resistance and anti-biofouling. At FIG. 8, reference letter (a) illustrates biocompatible electrode alloy (e.g., Pt—Ir, MP35N, etc.) to be nano-patterned by e-beam, ion-beam, nano-imprint lithography (NIL), extreme UV lithography (EUV), etc. At reference letter (b), illustrated is a positive or negative resist layer (e.g., PMMA, XEP520, HSQ, SU-8). At reference letter (c), illustrated is a patterned periodic or non-periodic nanopore array (e.g., 50-200 nm diameter), round or squared or other shape. Aspect ratio of e.g., 5-10, by various lithography processes. Nano-sheet array is also possible. At reference letter (d), illustrated is a low-pressure sputter deposit of Pt—Ir, MP35N, etc. At reference letter (e), illustrated is a high-pressure deposit of Pt—Ir, MP35N, etc., deep into a cavity. At reference letter (f), illustrated is a periodic array of taller, high aspect ratio protruding nanopillars, nanowires, nanosheets or nanotubes of electrode alloy after removal of resist.

Once the high-aspect-ratio nanoholes are produced, the holes are filled with metal electrode alloy material, e.g., by depositing the alloy into the pores or parallel gaps, preferably with the same composition as the substrate (e.g., Pt, Pt—Ir, MP35N, etc so as to minimize heterogeneity and adhesion issues) by e.g., high pressure sputtering, evaporation, or electrodeposition. The polymer resist is then dissolved away by solvent so as to expose the protruding array of nanopillars. Optionally Au-coated or Ti/Au coated on the nanopillar surface, according to the invention, for improved corrosion resistance and anti-biofouling.

### [E]. Disk Shape Shadow Mask for Creation of Elongated Nanopillar Structure

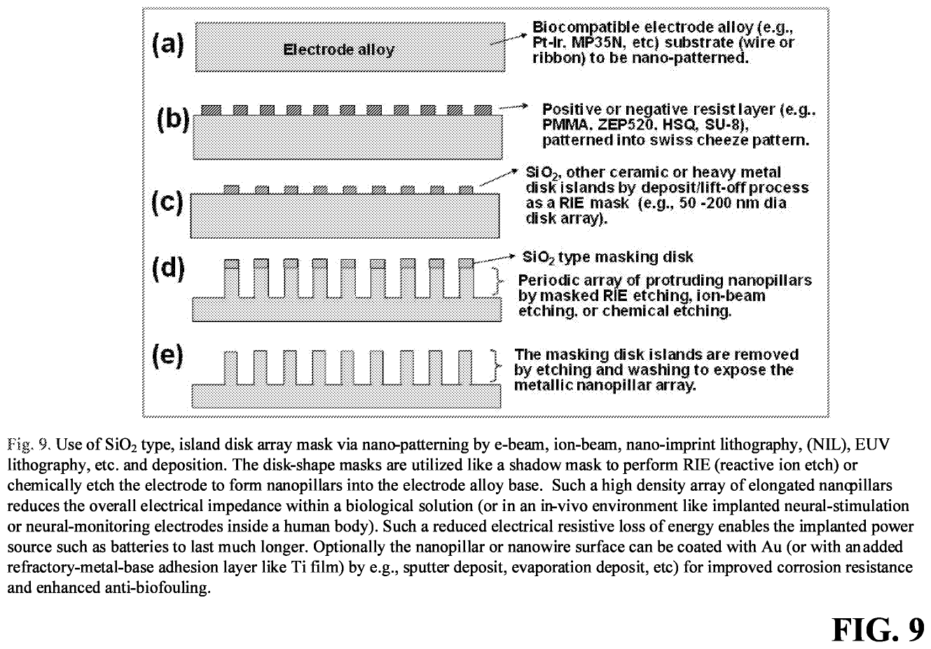

There are other variations of nanopatterning methods to produce a uniform and periodic nanopillar array. For example, as illustrated in FIG. 9, a disk shaped patterned mask (e.g., made of SiO₂, other ceramic or difficult-to-sputter metal layer) can be first prepared on the electrode surface, so as to allow RIE (reactive ion etch) or chemically etch the electrode to form nanopillars into the electrode alloy base. Such a high-density array of elongated nanopillars reduces the overall electrical impedance within a biological solution (or in an in-vivo environment like implanted neural-stimulation or neural-monitoring electrodes inside a human body). Such a reduced electrical resistive loss of energy enables the implanted power source such as batteries to last much longer, at least by 30% longer time, preferably by a factor of 2, more preferably by a factor of 5. Optionally the nanopillar or nanowire surface can be coated with Au or other noble metals such as Pt, Pd, Ir, Ru or their alloys (with an optionally added refractory-metal-base adhesion layer like Ti film) by e.g., sputter deposit, evaporation deposit, etc) for improved corrosion resistance and enhanced anti-biofouling.

Illustrated at FIG. 9 is a use of SiO₂ type, island disk array mask via nano-patterning by e-beam, ion-beam, nano-imprint lithography, (NIL), EUV lithography, etc. and deposition. The disk-shape masks are utilized like a shadow mask to perform RIE (reactive ion etch) or chemically etch the electrode to form nanopillars into the electrode alloy base. Such a high density array of elongated nanopillars reduces the overall electrical impedance within a biological solution (or in an in-vivo environment like implanted neural-stimulation or neural-monitoring electrodes inside a human body). Such a reduced electrical resistive loss of energy enables the implanted power source such as batteries to last much longer. Optionally the nanopillar or nanowire surface can be coated with Au (or with an added refractory-metal-base adhesion layer like Ti film) by e.g., sputter deposit, evaporation deposit, etc) for improved corrosion resistance and enhanced anti-biofouling.

At reference letter (a) of FIG. 9 illustrated is a biocompatible electrode alloy (e.g., Pt—Ir, MP35N, etc.) substrate (wire or ribbon) to be nano-patterned. At reference letter (b), illustrated is a positive or negative resist layer (e.g., PMMA, ZEP520, HSQ, SU-8), patterned into swiss cheeze pattern. At reference letter (c), illustrated is SiO₂, other ceramic or heavy metal disk islands by deposit/lift-off process as a RIE mask (e.g., 50-200 nm diameter disk array). At reference letter (d), illustrated is an SiO₂, masking disk. Also illustrated is a periodic array of protruding nanopillars by masked RIE etching, ion-beam etching, or chemical etching. At reference letter (e), illustrated is an exposed metallic nanopillar array, such that as the masking disk islands are removed by etching and washing, the metallic nanopillar array are exposed.

[F]. Nanopillars Growth Through Lithography-Nanopatterned Periodic Template Hole Array by Additive Electrochemical Deposition or Electroless Plating

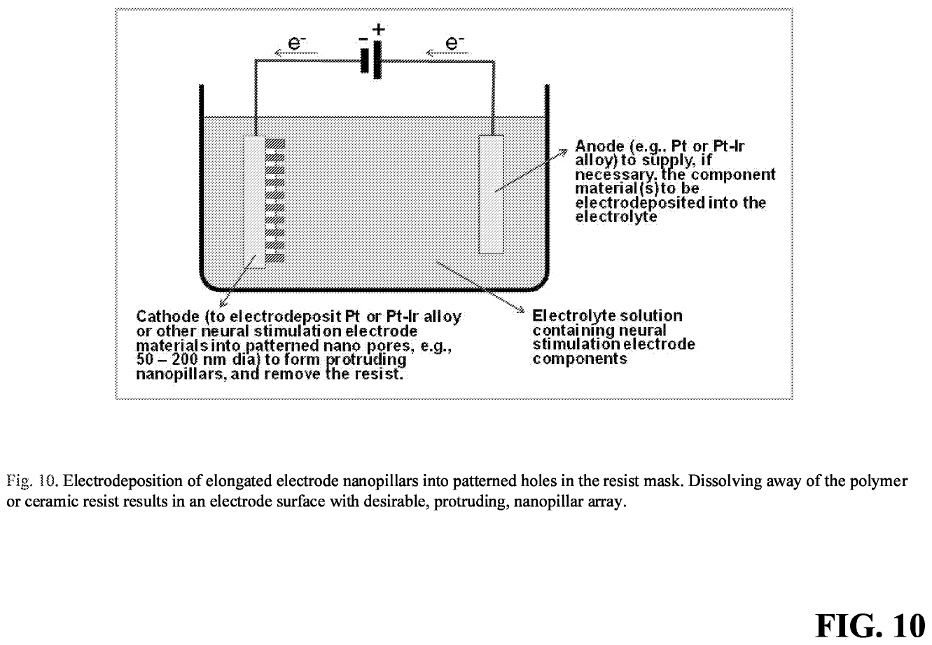

Shown in FIG. 10 is an alternative method to produce a uniform and periodic elongated nanopillars by utilizing electrodeposition of electrode material such as Pt or Pt—Ir alloy into the patterned holes in the resist mask. Dissolving away of the polymer or ceramic resist after the electrodeposition of the alloy material into the aligned nanopores results in an electrode surface with desirable, protruding nanopillar array. Similar approaches of producing high aspect ratio protruding structure for reduced electrode impedance properties are also available for lithography or shadow mask processed vertical nanopillars or nanoribbons, with either periodic or intentionally non-periodic nanostructures. For electrochemical deposition of Pt or Pt—Ir alloy, various electrolytes based on H₂PtCl₆, (NH₄)₂PtCl₆, H₃Pt(SO₃)₂OH or Pt(NO₂)₂(NH₃)₂ and similar Ir-containing chemicals (with a mix of Ptocontaining solution and Ir-containing solution) can be used for electrodeposition of Pt or Pt—Ir alloy. The electrodeposited Pt or Pt—Ir alloy can be solid or can have a nanoparticle based, nanoporous or microporous surface microstructures. High temperature heat treatment can be applied to the Pt or Pt—Ir deposit coating to consolidate the nanoparticle deposits and to reduce residual stress. In the case of porous Pt of Pt—Ir alloy electrodeposit, a preferred nanoporosity after sintering heat treatment is at least 30%, preferably at least 50%, even more preferably at least 70%. Furthermore, at FIG. 10 is an electrodeposition of elongated electrode nanopillars into patterned holes in the resist mask. Dissolving away of the polymer or ceramic resist results in an electrode surface with desirable, protruding, nanopillar array.

### [G]. Use of Seed Nanopillars for Growth of High-Aspect-Ratio Nanostructures

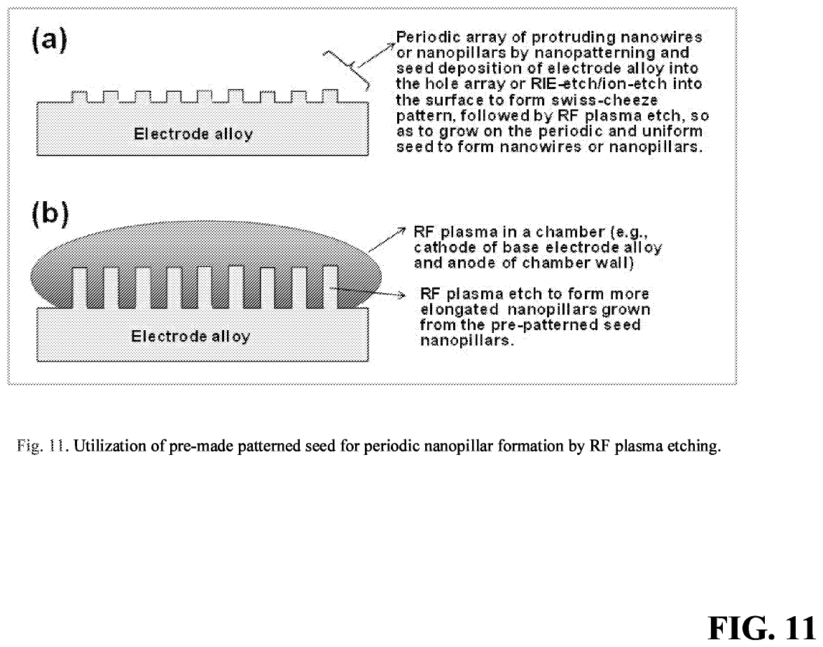

(1) Near-room-temperature, reactive-plasma-etch induced seed nanopillars. According to the invention, seed nanopillars (FIG. 11) can be provided on the electrode surface for easier build up of nanopillars (or related protruding nanostructures). Such seed nanopillars can be produced by nanopatterning of a mask island array followed by plasma etch (e.g., RIE reactive ion etch) or chemical etch. On subsequent high temperature plasma etching using RF, microwave or ICP plasma etching, the nanopillar becomes longer due to the presence of the guiding seed structure. Furthermore, at FIG. 11 illustrated is a utilization of pre-made patterned seed for periodic nanopillar formation by RF plasma etching. Furthermore, at reference letter (a) is a periodic array of protruding nanowires or nanopillars by nanopatterning and seed deposition of electrode alloy into the hole array or RIE-etch/ion-etch into the surface to form swiss-cheeze pattern, followed by RF plasma etch, so as to grow on the periodic and uniform seed form nanowires or nanopillars. At reference letter (b) is an RF plasma in a chamber (e.g., cathode of bass electrode alloy and anode of chamber wall). Furthermore is an RF plasma etch to form more elongated nanopillars grown from the pre-patterned seed nanopillars.

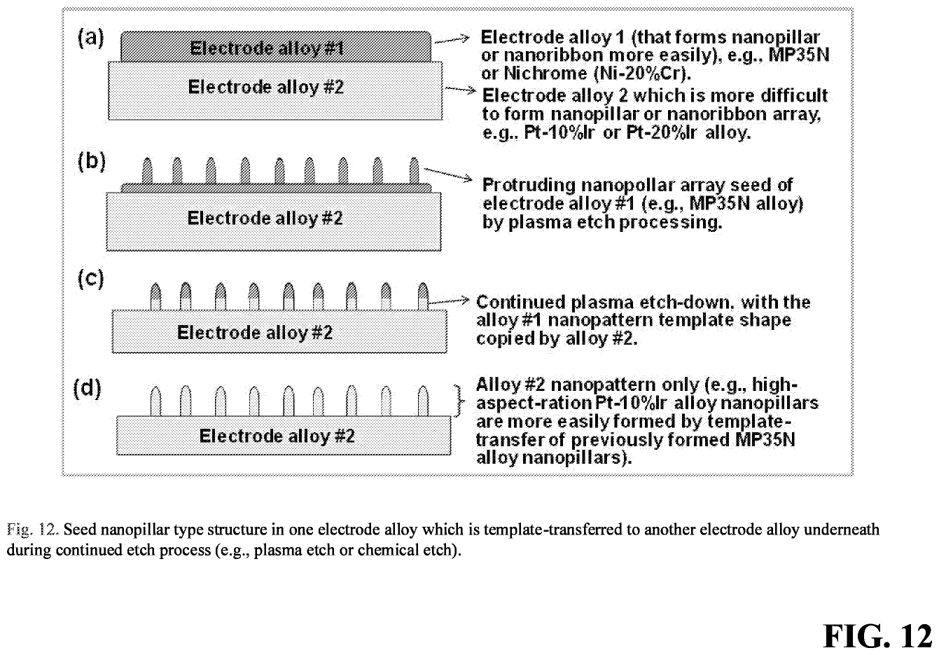

(2) Two-layered electrode with plasma-etch induced seed nanopillars—The efficiency of nanopillar formation by RF plasma etching or ICP plasma etching varies from material to material. For example, MP35N type electrode alloy or Nichrome (Ni-20% Cr) alloy responds much better to the RF processing than Pt—Ir electrode alloy in terms of resultant height and vertical aspect ratio of nanopillars or nanoribbons, with more uniform distribution of nanostructures observed. Therefore, one of the desirable process variations is to utilize MP35N or Nichrome alloy layer pre-deposited on Pt—Ir electrode alloy as a sacrificial alloy, and proceed with RF texturing to create desirably-shaped MP35N or Nichrome nanopillars or nanoribbons first, and then continue with RF plasma etching into the bottom layer Pt—Ir alloy matrix, so that the MP35N or Nichrome alloy seed layer nanostructure is continued and transferred into the Pt—Ir alloy underneath upon continued plasma etching. This approach is schematically illustrated in FIG. 12. Furthermore, FIG. 12 illustrates a seed nanopillar type structure in one electrode alloy which is template-transferred to another electrode alloy underneath during continued etch process (e.g., plasma etch or chemical etch).

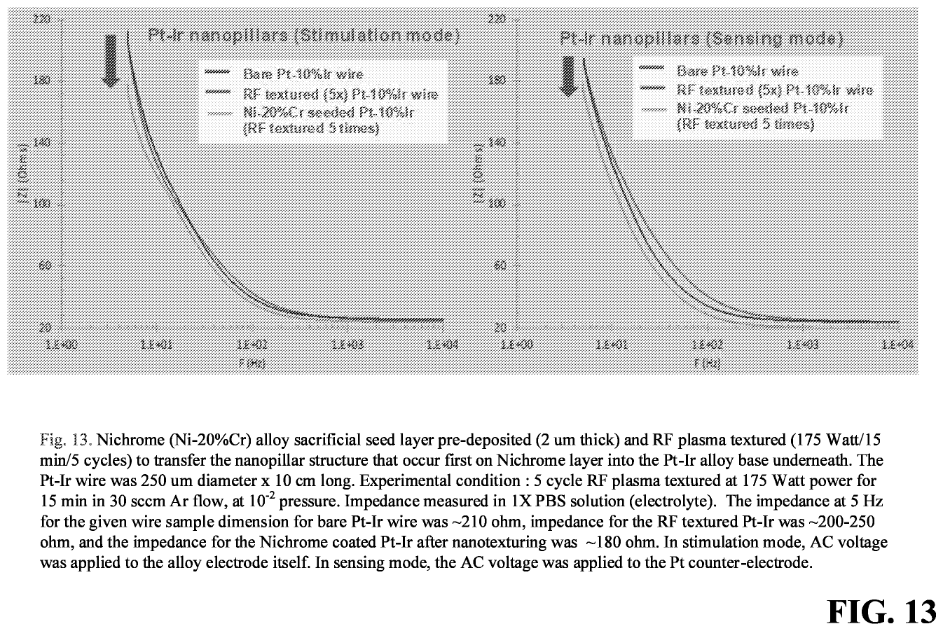

Shown in FIG. 13 is the experimental impedance measurement data, which shows that the use of sacrificial Nichrome seed layer placed on top of Pt—Ir electrode alloy results in improved properties as the impedance is lowered by such a nanopattern transfer processing. At FIG. 13, illustrated is a Nichrome (Ni-20% Cr) alloy sacrificial seed layer pre-deposited (2 um thick) and RF plasma textured (175 Watt/15 min/5 cycles) to transfer the nanopillar structure that occur first on Nichrome layer into the Pt—Ir alloy base underneath. The Pt—Ir wire was 250 um diameter×10 cm long. Experimental condition: 5 cycle RF plasma textured at 175 Watt power for 15 min in 30 sccm Ar flow, at 10⁻² pressure. Impedance measured in 1× PBS solution (electrolyte). The impedance at 5 Hz for the given wire sample dimension for bare Pt—Ir wire was ˜210 ohm, impedance for the RF textured Pt—Ir was ˜200-250 ohm, and the impedance for the Nichrome coated Pt—Ir after nanotexturing was ˜180 ohm. In stimulation mode, AC voltage was applied to the alloy electrode itself. In sensing mode, the AC voltage was applied to the Pt counter-electrode.

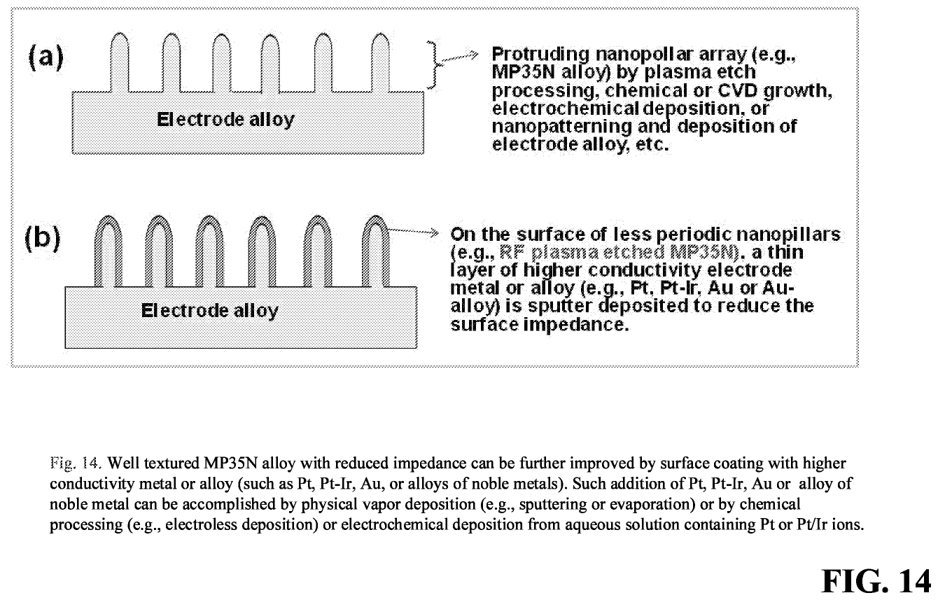

(3) High conductivity film coating on nanopillared electrode alloy for lowered impedance. While MP35N alloy responds better than Pt—Ir electrode alloy in terms of developing high aspect ratio nanopillar type structure (desirable for lowering of the impedance), MP35N has inherently higher electrical resistivity and tends to form thin surface natural oxide, so as to yield generally higher impedance values than those for identical dimension Pt—Ir alloy electrodes. Therefore, this invention teaches a new approach of adding a highly conductive layer coating of noble metal (such as Pt, Pt—Ir, Pd, Ru, Au and their alloys, e.g., 5-100 nm thick coating by sputtering, evaporation, ion deposition, chemical, electroless or electrolytic deposition), so as to reduce the interfacial impedance. This approach is schematically illustrated in FIG. 14.

Furthermore, FIG. 14 illustrates a well textured MP35N alloy with reduced impedance can be further improved by surface coating with higher conductivity metal or alloy (such as Pt, Pt—Ir, Au, or alloys of noble metals). Such addition of Pt, Pt—Ir, Au or alloy of noble metal can be accomplished by physical vapor deposition (e.g., sputtering or evaporation) or by chemical processing (e.g., electroless deposition) or electrochemical deposition from aqueous solution containing Pt or Pt/Ir ions.

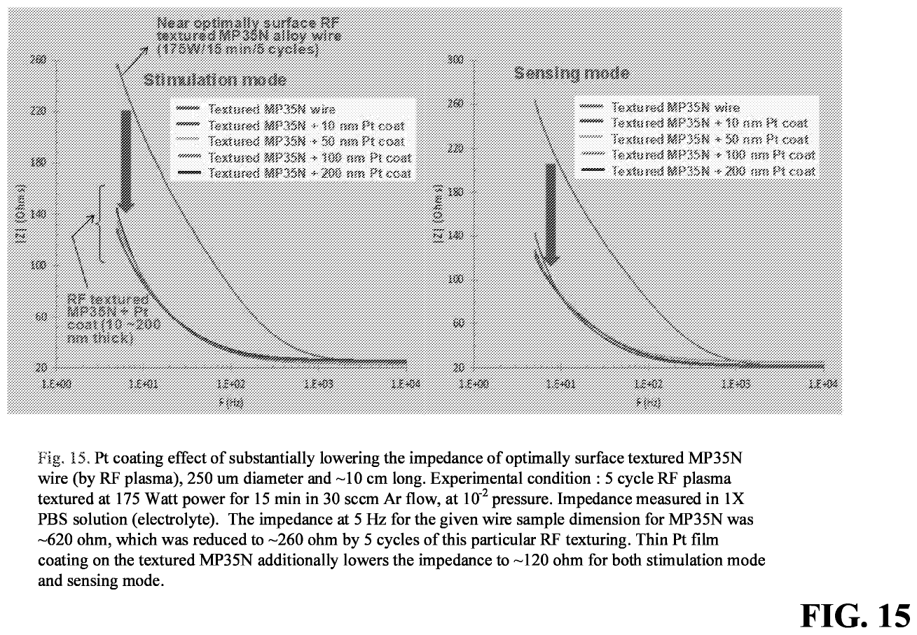

A sample processed this way (Pt coating on optimally surface textured MP35N wire (by RF plasma), 250 um diameter and ˜10 cm long, resulted in a substantial reduction of impedance as shown in FIG. 15. The experimental condition includes 5 cycle RF plasma texturing at 175 Watt power for 15 min in 30 sccm Ar flow, at 10⁻² pressure. The impedance was measured in a phosphate-buffered saline (PBS) solution as the electrolyte. The impedance at 5 Hz for the given wire sample dimension for MP35N was ˜620 ohm, which was reduced to ˜260 ohm by 5 cycles of this particular RF texturing. Thin Pt film coating on the textured MP35N additionally lowers the impedance to ˜120 ohm for both stimulation mode and sensing mode, as can be seen in FIG. 15.

For electroless deposition of Pt or Pt—Ir alloy on the surface of base alloy nanopillar or nanostructure surface, organoplatinum precursor such as cis-dichlorobis(styrene)platinum(II) dissolved in a solvent like toluene can be utilized, with accelerated reaction enabled by heating of the electroless plating solution (e.g., at 50-200° C., preferably at 70-150° C.). The electroless deposited films can be 5 nm to 1,000 nm thick, preferably 10-100 nm thick, and can have either continuous film morphology or nanoporous morphology depending on the process conditions. For Pt—Ir coating a mixed organo-(platinum-iridium) compound can be utilized. Nanoporous Pt or nanoporous Pt—Ir coating (e.g., made of nanoparticles or 0.5-20 nm size, preferably 2-10 nm size) is preferred than a smooth film as the nanoscale surface roughness and porosity further lowers the impedance. Several different methods of Pt electroless deposition can be utilized. Another example of Pt electroless deposition is to utilize an aqueous solution of HClO₄ which contains K₂PtCl₆. The Pt or Pt—Ir deposit can be lightly annealed at high temperature (e.g., 200-800° C. for 1 min to 1 hr) to partially sinter consolidate the nanoparticles for enhanced mechanical robustness, and also to reduce residual stress in the deposited film for more reliable coating adhesion. A preferred nanoporosity after sintering heat treatment is at least 30%, preferably at least 50%, even more preferably at least 70%.

Electrochemical deposition of Pt or Pt—Ir alloy can also be utilized to coat the nanopillar type shaped electrode base structure. Various electrolytes based on H₂PtCl₆, (NH₄)₂PtCl₆, H₃Pt(SO₃)₂OH or Pt(NO₂)₂(NH₃)₂ and similar Ir-containing chemicals can be used for electrodeposition, preferably with nanoparticle based, nanoporous or microporous surface microstructures. High temperature heat treatment can be applied to the Pt or Pt—Ir deposit coating to consolidate the nanoparticle deposits and to reduce residual stress. A preferred nanoporosity after sintering heat treatment is at least 30%, preferably at least 50%, even more preferably at least 70%.

Furthermore, at FIG. 15. Is a Pt coating effect of substantially lowering the impedance of optimally surface textured MP35N wire (by RF plasma), 250 um diameter and ˜10 cm long. Experimental condition: 5 cycle RF plasma textured at 175 Watt power for 15 min in 30 sccm Ar flow, at 10⁻² pressure. Impedance measured in 1× PBS solution (electrolyte). The impedance at 5 Hz for the given wire sample dimension for MP35N was ˜620 ohm, which was reduced to ˜260 ohm by 5 cycles of this particular RF texturing. Thin Pt film coating on the textured MP35N additionally lowers the impedance to ˜120 ohm for both stimulation mode and sensing mode.

### [H]. Pulse Sensing Data

For feedback based electrical stimulation, a high-resolution, reliable sensing of body neural signals such as ECAP (electrically evoked compound action potential) signals is important. Closed-loop electrical stimulation systems such as spinal cord stimulation (SCS) or deep brain stimulation (DBS) are promising as they can relieve the clinical burden of controlling electrical stimulation parameter for improved electrical stimulation based treatments. In such a system, A feedback signal can be utilized to automatically adjust and control stimulation process specifics in order to optimize the efficacy of stimulation treatment.

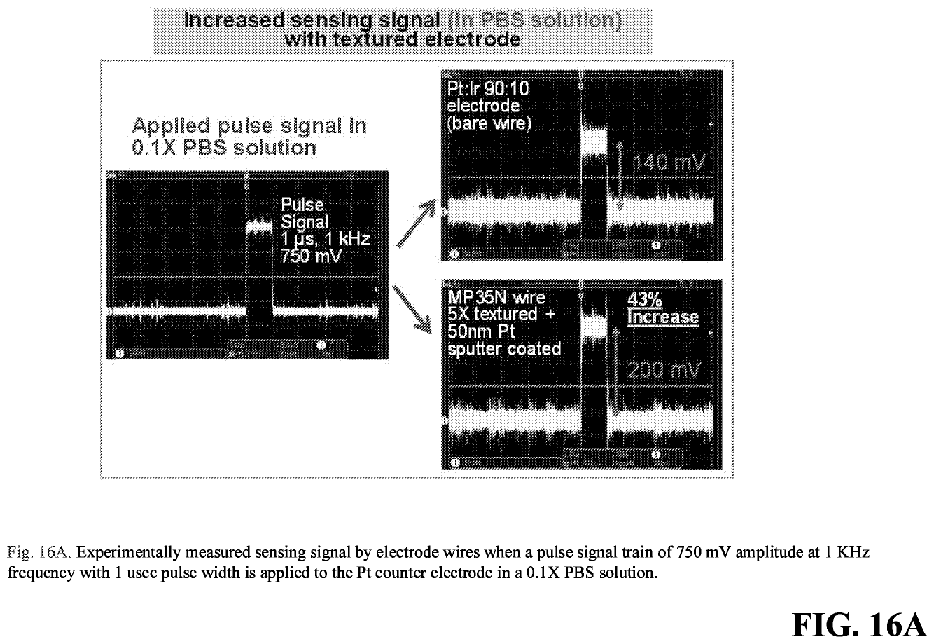

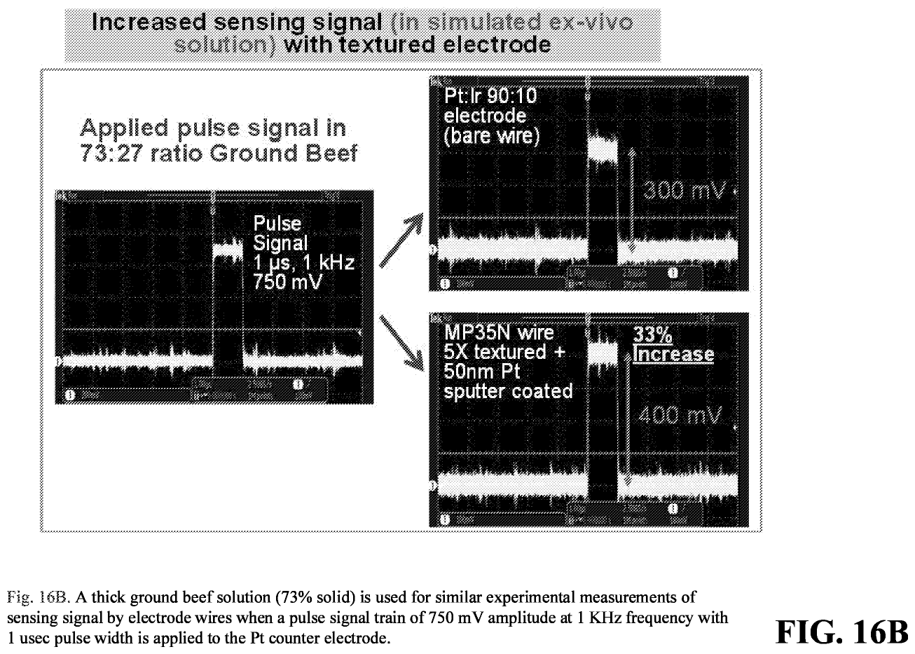

Shown in FIG. 16A is the experimentally measured sensing signal by electrode wires when a pulse signal train of 750 mV amplitude at 1 KHz frequency with 1 usec pulse width is applied to the Pt counter electrode in a 0.1× PBS solution. Compared to the standard Pt-10% Ir electrode wire, the nanotextured MP35N alloy wire (5 times RF plasma textured for taller nanopillar formation followed by 50 nm Pt coating by sputtering), which represents the example schematics shown in FIG. 14, produces a noticeably improved sense signal, increased by 43% in this example. Shown in FIG. 16B is also experimentally measured sensing signals under similar conditions by electrode wires but in a thick (73% volume) ground beef solution to simulate ex-vivo environment, closer to the in-vivo environment than the simple PBS (phosphate-buffered saline) solution. In this case, the sensing signal amplitude is improved by 33%.

At FIG. 16B, illustrated is a thick ground beef solution (73% solid) is used for similar experimental measurements of sensing signal by electrode wires when a pulse signal train of 750 mV amplitude at 1 KHz frequency with 1 usec pulse width is applied to the Pt counter electrode.

### [I]. Further Elongation of Nanopillars by Additive Electrochemical Deposition or Electroless Plating

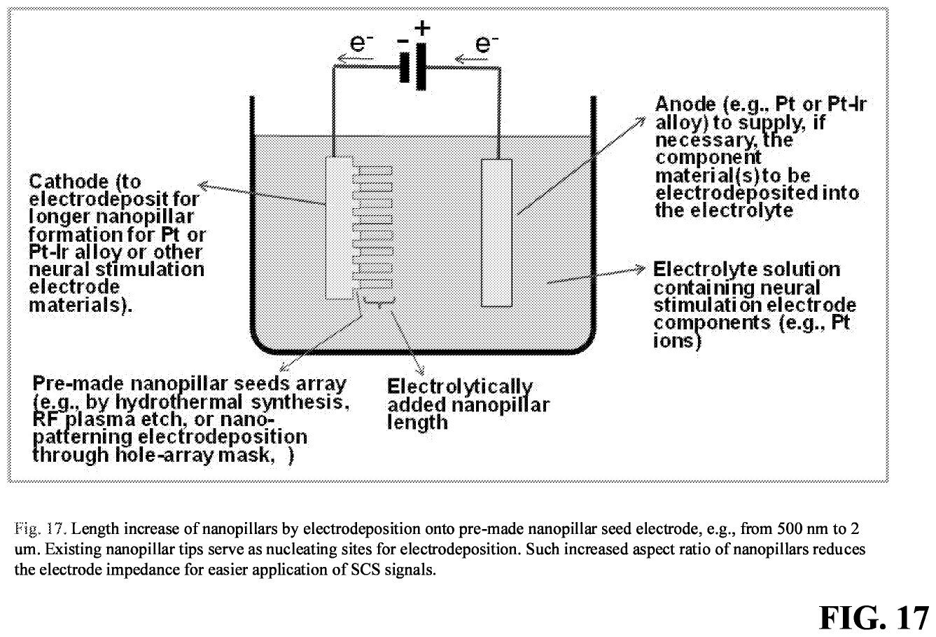

Uniform and periodic nanopillar array can be obtained by RF plasma process, according to the invention, if one uses a seed array of periodically positioned short nanopillars, e.g., obtained by nanopatterning process, which is illustrated e.g., in FIG. 6, FIG. 7, FIG. 11 or FIG. 12. As the length of the nanopillars plays an important role of dictating the degree of impedance reduction, longer nanopillars are preferred. It is highly desirable to find a method of increasing the nanopillar height. One such method, according to the invention, is to utilize electrochemical deposition on previously grown nanopillars such as by hydrothermal method, template growth (using AAO type patterned membrane), RF plasma etch process, and so forth. One example is shown in FIG. 17 in which a length increase of nanopillars by electrodeposition onto pre-made nanopillar electrode (e.g., from 500 nm length to an increased length of 2 um) is illustrated. Existing nanopillar tips (e.g., prepared by nanopatterning, RF plasma etching, microwave plasma etching, inductively coupled plasma (ICP) etching, hydrothermal growth, or nanopillar growth guided through a channeled mask) serve as nucleating sites for electrodeposition of Pt or Pt—Ir alloy on seed nanopillars of identical or similar metallic composition. For electrodeposition of Pt or Pt—Ir alloy can be performed using various electrolytes such as based on H₂PtCl₆, (NH₄)₂PtCl₆, H₃Pt(SO₃)₂OH or Pt(NO₂)₂(NH₃)₂, and similar Ir-containing chemicals (with a mix of Ptocontaining solution and Ir-containing solution) can be used for electrodeposition, preferably with nanoparticle based, nanoporous or microporous surface microstructures. High temperature heat treatment can be applied to the Pt or Pt—Ir deposit coating to consolidate the nanoparticle deposits and to reduce residual stress. A preferred nanoporosity after sintering heat treatment is at least 30%, preferably at least 50%, even more preferably at least 70%.

Such increased aspect ratio of nanopillars reduces the electrode impedance for easier application of SCS signals. Electrolytically induced nanopillar length increase occurs on cathode where the electrodeposition occurs onto the tips of Pt or Pt—Ir nanopillars.

### [J]. Reduction of Eddy Current Loss at High Frequency Electrical Stimulation and Sensing

Electrical stimulation such as for SCS can use either low frequencies of e.g., <1 KHz or higher frequencies of 10 KHz or higher, with the latter utilized to eliminate or reduce the paresthesia (e.g., tingling sensations). When the electrical stimulation electrode is operated at a higher frequency such as 5 to 20 KHz, the higher frequency tends to induce eddy current loss on the conductive electrode surface. Even at lower frequency stimulations, if the current pulse applied is a square-loop shape, the steep rise time of the applied current pulse may be considered a high frequency in nature. In order to reduce such eddy current loss on high frequency or square-loop electrical stimulations, it is desirable to subdivide the electrode alloy into smaller diameters or smaller grain structures. It is also helpful to make the surface of the nanopillar to exhibit an ultra-fine grain size with higher electrical resistance to minimize the eddy current. According to the invention, at least four different innovative approaches are presented to reduce the eddy current loss.

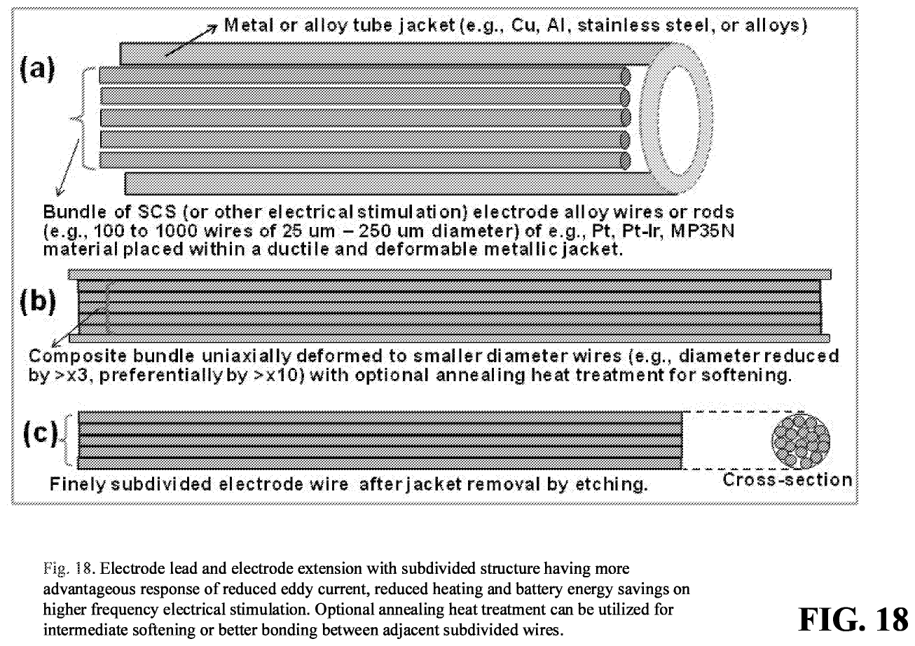

(a) Reduce the alloy lead wire diameter—Shown in FIG. 18 is an electrode lead extension wire with subdivided structure having a more advantageous response of reduced eddy current, reduced heating, and battery energy savings on higher frequency electrical stimulation. Optional annealing heat treatment can be utilized for intermediate softening or better bonding between adjacent subdivided wires. A bundle of SCS (or other electrical stimulation) electrode alloy wires or rods (e.g., 100 to 1000 wires of 25 um-100 um diameter) of e.g., Pt, Pt—Ir, MP35N material is placed within a ductile and deformable metallic jacket, such as Cu, Al, stainless steel, or other alloys. The composite bundle inside a jacket is uniaxially deformed (e.g., by swaging, extrusion, rod drawing or wire drawing) to smaller diameter wires (e.g., diameter reduced by a factor of >×3, preferentially by >×10) with optional annealing heat treatment(s) for softening. The desirable final dimension of this multi-strand subdivided extension wire is 25-500 um, preferably 50-100 um. The electrical resistivity of this multi-strand wire is at least 50% higher, preferably at least ×2 higher than that of a solid wire having an identical volume.

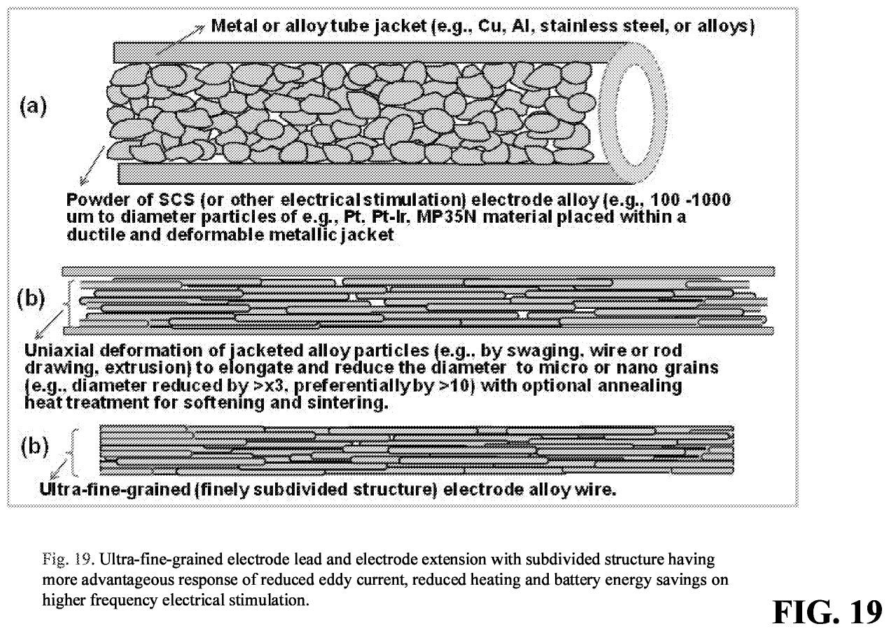

(b) Reduce the alloy particle diameter—Instead of subdividing the lead extension wire (or electrode) into smaller diameter wire bundles, the starting material can be powders of the electrode material (such as Pt, Pt—Ir, or MP35N) placed in a metal tube jacket. The composite material is then uniaxially deformed (e.g., by swaging, extrusion, rod drawing or wire drawing) to smaller diameter wires (e.g., the overall diameter reduced by a factor of >×3, preferentially by >×10) with optional annealing heat treatment(s) for softening, as shown in FIG. 19. With such a processing, an ultra-fine-grained material with subdivided structure is obtained, which exhibits a more advantageous response of reduced eddy current, reduced heating and battery energy savings on higher frequency electrical stimulation. The reduced eddy current also allows to use higher frequency (e.g., increased by a factor of ×2-100) electrical signal pulsing if needed.

The desirable final dimension of this multi-strand subdivided extension wire is 25-500 um, preferably 50-100 um. The grain elongation aspect ratio is at least 2, preferably at least 5, more preferably at least 10. The electrical resistivity of this multi-strand wire is at least 50% higher, preferably at least ×2 higher than that of a solid wire having an identical volume. The average diameter of the elongated grains is typically less than 20 um, preferably less than 5 um, even more preferably less than 1 um. Sub-dividing of extension wire with smaller size strands further reduces the eddy current loss (by a factor of ×2, preferably ×5) and allows higher frequency operations of pulsing to the neural receptors for pain relief. The grain size within each strand is also reduced when the subdivided wire diameter is made smaller.

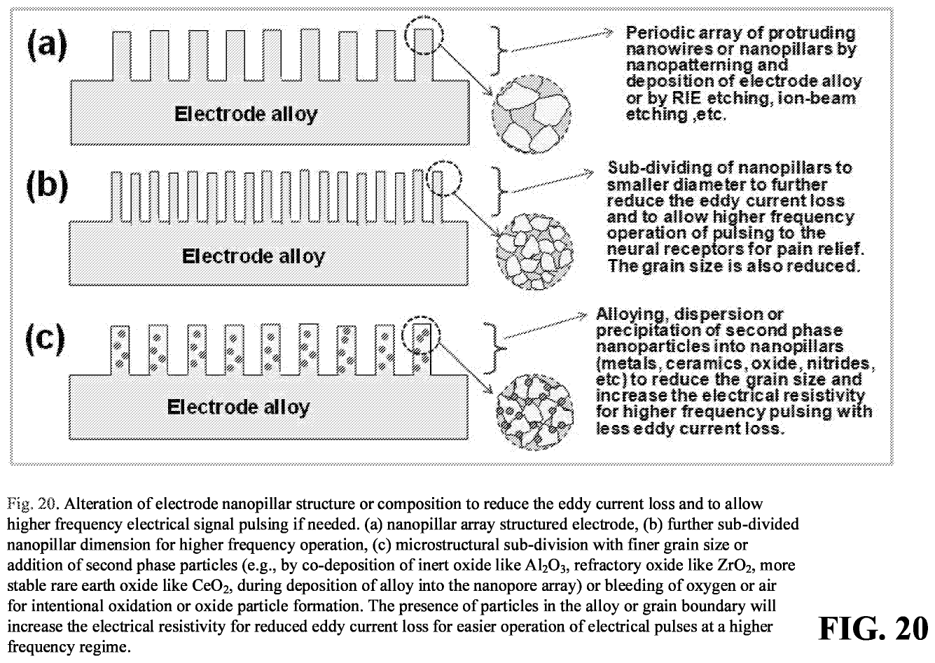

(c) Make nanopillar to have a metal-oxide composite structure—Shown in FIG. 20 is an altered electrode nanopillar structure or composition to reduce the eddy current loss and to allow higher frequency electrical signal pulsing if needed. A periodic array of protruding nanopillars is formed by nanopatterning and deposition of electrode alloy or by RIE etching, ion-beam etching, etc (FIG. 20(a)). In FIG. 20(b), a subdivided (smaller diameter) nanopillar structure is illustrated. Alteration of electrode nanopillar structure or composition is performed to reduce the eddy current loss and to allow higher frequency (e.g., ×2-100 increased) electrical signal pulsing if needed. Sub-dividing of nanopillars with a smaller grain size further reduces the eddy current loss (by a factor of ×2, preferably ×5) and allows higher frequency operations of pulsing to the neural receptors for pain relief. The grain size within each nanopillar is also reduced when the nanopillar diameter is made smaller.

When comparing the structure of FIG. 20(a) having a larger diameter nanopillar array structured electrode with the structure of FIG. 20(b) showing a further diameter reduced nanopillar dimension, the smaller diameter nanopillar structure of FIG. 20(b) reduces the eddy current loss (by a factor of ×2, preferably ×5) which is more desirable for higher frequency operations. In FIG. 20(c), microstructural sub-division is schematically illustrated, with finer grain size or addition of second phase particles (e.g., by co-deposition of inert oxide like Al₂O₃, refractory oxide like ZrO₂, more stable rare earth oxide like CeO₂, during deposition of the alloy into the nanopore array) or by bleeding of oxygen or air for intentional oxidation or oxide particle formation. The presence of particles (desirably 2-200 nm diameter, preferably 5-100 nm) in the alloy or grain boundary will increase the electrical resistivity and also make the grain size smaller for reduced eddy current loss for easier operation of electrical pulses at a higher frequency pulsing stimulation regime.

The desirable grain size dimension is at least 50%, preferably by a factor of ×2, more preferably by a factor of ×5 reduced as compared to the identical material without the grain refinement. The grain elongation aspect ratio is at least 2, preferably at least 5, more preferably at least 10. The electrical resistivity of this multi-strand wire is at least 50% higher, preferably at least ×2 higher than that of a solid wire having an identical volume. The average diameter of the elongated grains is typically less than 20 um, preferably less than 5 um, even more preferably less than 1 um.

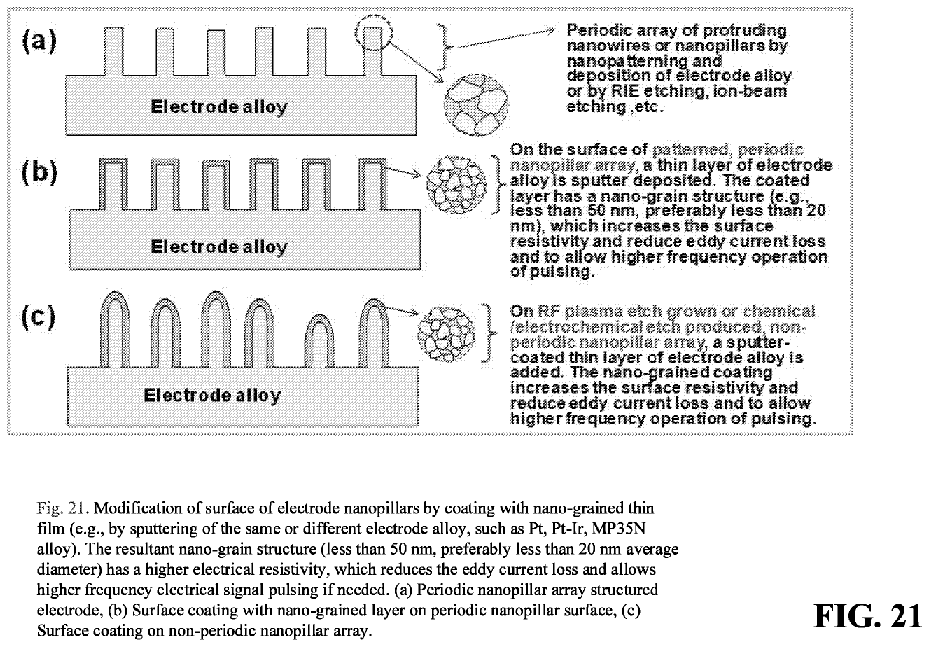

(d) Deposit ultra-fine grain size surface coating on the nanopillar surface—Shown in FIG. 21 is a modification of surface of electrode nanopillars by coating with nano-grained thin film (e.g., by sputtering or evaporation deposition of the same or different electrode alloy, such as Pt, Pt—Ir, MP35N alloy) on a periodic nanopillar array, FIG. 21(b). The resultant nano-grain structure on the surface (with a dimension of less than 100 nm, preferably less than 50 nm, even more preferably less than 20 nm average diameter) has a higher electrical resistivity, which reduces the eddy current loss (by at least 50%, preferably by a factor of ×2, more preferably by a factor of ×5) and allows higher frequency electrical signal pulsing if needed. Such a nanograined, higher-resistivity coating can also be applied onto non-periodic nanopillars, such as formed by hydrothermal synthesis, by template-guided nanopillar growth, or by RF plasma etching, as illustrated in FIG. 21(c), in order to utilize the benefit of nanograins having a reduced eddy current loss. The electrical resistivity of this nanograin-coated electrode material is at least 50% higher, preferably at least ×2 higher than that of a nanopillar structure having an identical geometry. The average diameter of the surface grains is typically less than 20 um, preferably less than 5 um, even more preferably less than 1 um.

An alternative approach to increase the electrical resistivity and reduce the eddy current loss is to apply a thin coating of conductive yet higher resistivity alloy (e.g., Nichrome alloy, alloys of Pt such as Pt—Ir, Pt—Au, or other noble metal alloys), or aqueous-solution-stable conductive oxide such as ferrites, perovskite Mn-oxide, indium tin oxide, fluorinated tin oxide. Some conductive carbide or conductive nitride coating is also possible.

### [K]. IrO₂ Coating for Reduced Impedance

The nanotextured electrode alloy such as MP35N or Pt—Ir can be coated with a thin layer of IrO₂, which has been found to reduce the electrode impedance and enhance electrical signal sensing (such as ECAP signals). According to the invention, the following processing nethods can be utilized to introduce a thin IrO₂ layer on the electrode surface.

(1). Use of natural oxidation or high temperature intentional oxidation. (i) Pt-20% Ir alloy wire surface, (ii) Pt-10% Ir alloy can be heat treated, for example at 200-900° C. for 1 minute to 48 hrs, in air or in oxygen-containing atmosphere, or (iii) pre-coat any Pt-containing alloy or MP35N alloy with a thin Ir metal film (e.g., 0.5-30 nm, preferably 1-10 nm thickness, by sputtering or evaporation, or electrochemical deposition), followed by oxidizing heat treatment to create an IrO₂ on electrode surface.

(2) PVD, CVD or electrochemical deposition of IrO₂. A thin layer of IrO₂ (desirably 0.5-30 nm, preferably 1-10 nm thickness) can be deposited on the MP35N, Pt—Ir or other neural stimulation electrode or sensing electrode surface by physical vapor deposition (PVD) such as sputtering, e-beam evaporation, or chemical processing (such as chemical vapor deposition (CVD) or electrolytic deposition)

### [L]. Neural Electrode Shape Alterations

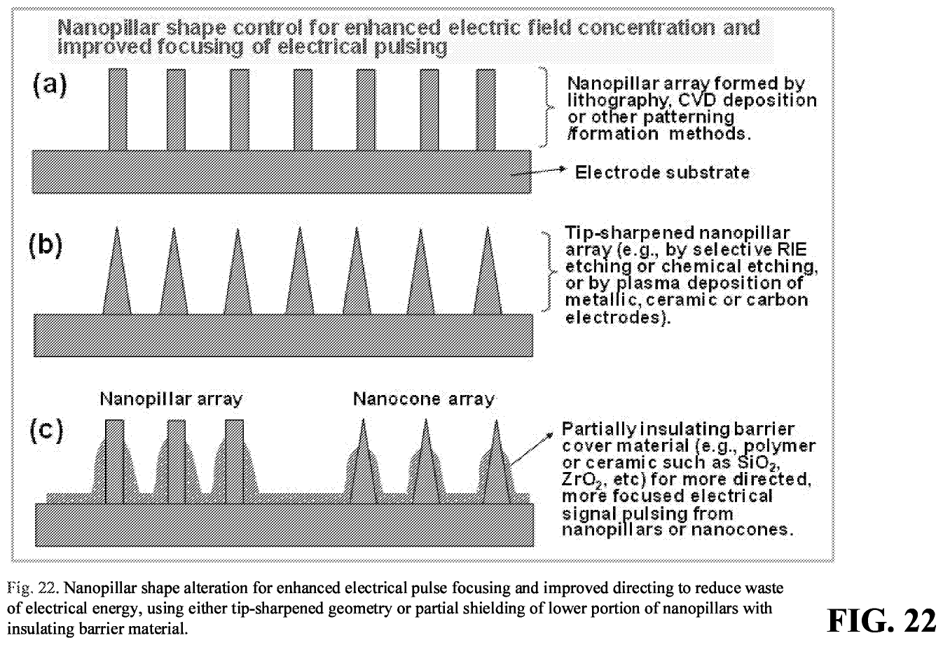

Shown in FIG. 22 is an example of nanopillar shape alteration method and structure for enhanced electrical pulse focusing and improved directing to reduce waste of electrical energy, using either tip-sharpened geometry or partial shielding of lower portion of nanopillars with insulating barrier material. Nanopillar array formed by lithography, CVD deposition or other patterning or growth methods. Either on the surface of regular nanopillars or on tip-sharpened nanopillars (e.g., by selective RIE etching or chemical etching, or by plasma deposition of metallic, ceramic or carbon electrodes), a partially insulating barrier cover material (e.g., polymer or ceramic such as SiO₂, ZrO₂, etc) is applied so that the tip region is selectively exposed for electrical pulse release, which enables a more directed, more focused electrical signal pulsing from the nanopillars or nanocones. Si-based micropillars can be repeatedly oxidized and chemically etched with gradual sharpening of the tip region, as etching kinetics are usually faster near the end of the protruding tip. The Si sharp tip can be electrically conductive via previous doping. Alternatively such a sharp tip of Si, SiO₂, ZrO₂, etc can be coated with conductive metals such as Pt, Pt—Ir, Pd, Ru, Au, and their alloys, MP35N, stainless steel and so forth (with adhesion layer of Ti or Cr pre-deposited at the interface).

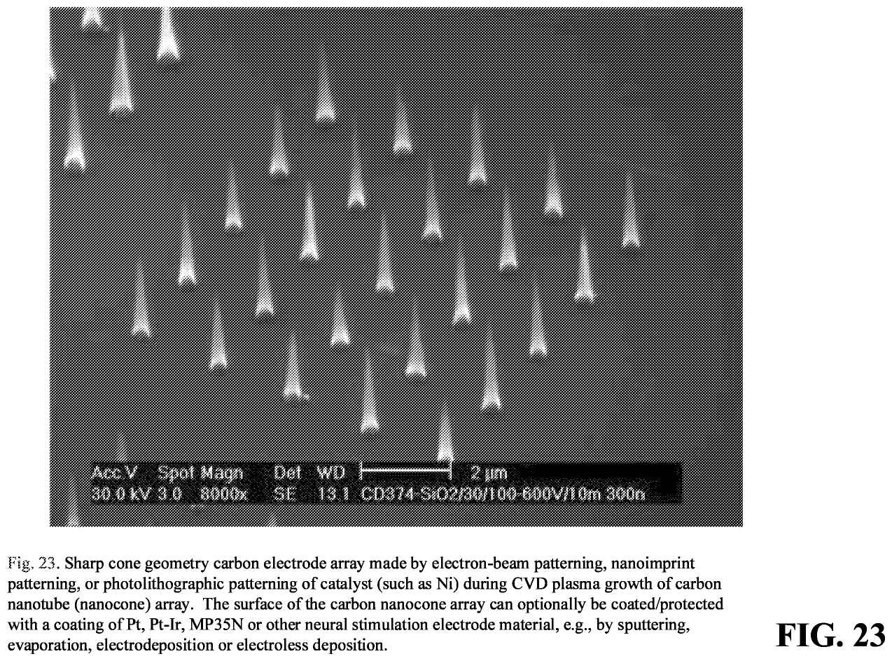

Another example of tip-sharpened nanopillar array structure is shown in FIG. 23. Sharp cone geometry carbon electrode array is made by electron-beam patterning, nanoimprint patterning, or photolithographic patterning of catalyst (such as Ni) prior to the chemical vapor deposition (CVD) plasma growth of carbon nanotube (nanocone) array. The surface of the carbon nanocone array can optionally be coated/protected with a coating of Pt, Pt—Ir, MP35N or other neural stimulation electrode material, e.g., by sputtering, evaporation, electrodeposition or electroless deposition.

### [M]. Anti-Biofouling Structure

Coating of biological or biomedical devices/materials with certain polymers such as PTFE (Polytetrafluoroethylene, also known as teflon) or PEG (polyethylene glycol) helps to minimize biofouling. However, application of these electrically insulating polymer materials as a coating on the metallic electrode surface would block the electrical pulse signal to make the electrode ineffective. In order to circumvent this problem, a unique electrode structure of having the nanopillar tips coated by PTFE or PEG but allowing the sidewall of the nanopillars to electrically conduct and send current pulses to the intended target area for electrical stimulation in human body has been developed according to the invention.

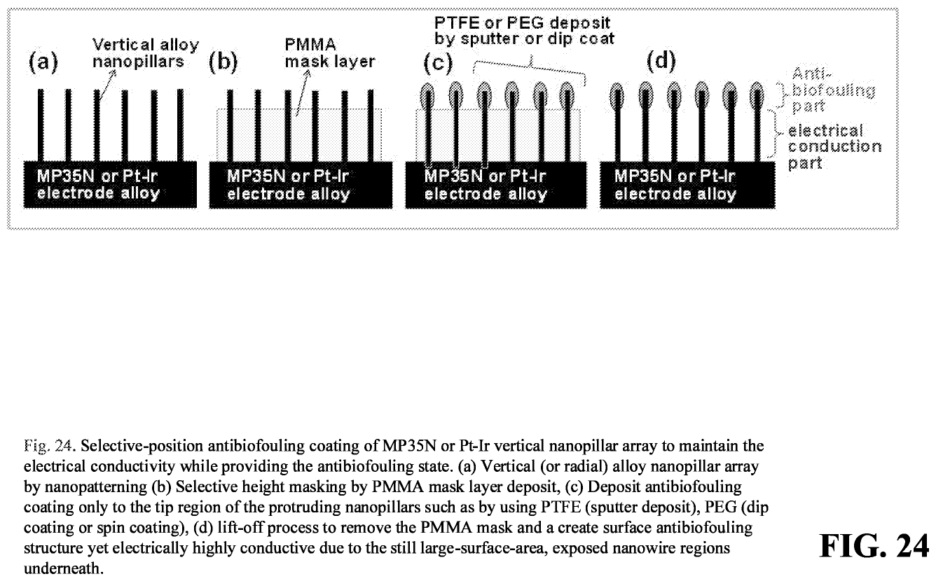

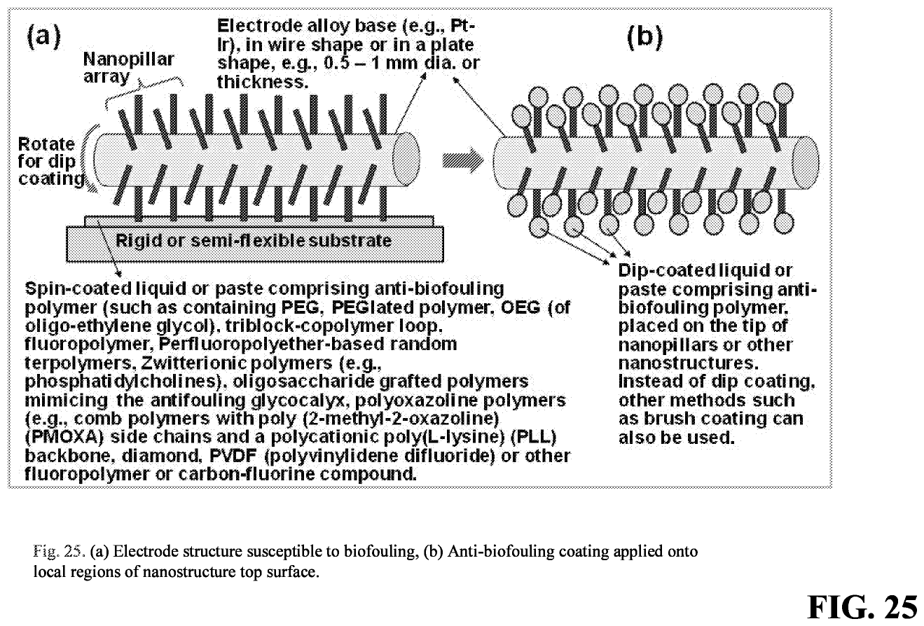

Referring to the drawings, FIG. 24 schematically illustrates such an invention. First, a nanopillar array on electrode materials (such as MP35N or Pt—Ir) is prepared (FIG. 24(a)), e.g., by nanopatterning, RF plasma etching, hydrothermal growth or template nanopillar growth. A Selective tip height masking by PMMA (polymethyl methacrylate) mask layer deposition is carried out (FIG. 24(b)). An example processing is to have the PMMA film spin coated on the substrate with nanopillars to fill the trenches and to cover the nanopillar, then subsequently baked on a hotplate at 115° C. for 90 sec. to vaporize the solvent in PMMA. The substrate is then re-etched by RIE to remove the “overfilled” regions and the tip regions (300˜500 nm) of the nanowires on the substrate. ˜50 nm Teflon (using PTFE target) can be sputtered on the pre-treated substrate. Teflon coated remains only on the tip regions selectively after a lift-off process using acetone.

The nanopillars are then selective-position antibiofouling coated with PTFE (polytetrafluoroethylene, often called Teflon) or PEG (polyethylene glycol) on the nanopillar tips (FIG. 24(c)), e.g., by dip coating. As biofouling cells or proteins have a certain size dimension, they cannot easily penetrate through the nanoscale gaps between the adjacent nanopillars. A lift-off type process is carried out to remove the PMMA mask (FIG. 24(d)) and create a surface antibiofouling structure yet electrically highly conductive due to the still large-surface-area, exposed nanowire regions underneath.

After the anti-biofouling teflon coating is applied to the upper portion, e.g., ¼ of the nanopillar length, the impedance can be increased slightly, but the remaining portion, e.g., ¾ height of the metallic nanopillar is still exposed to carry out the electrical pulsing operation through the medium in the human body toward the targeted regions (e.g., neural positions to be stimulated in the spine). Thus, the antibiofouling insulating coating can be added without sacrificing much of the original electrode conductivity. Structurally, the desirable anti-biofouling neural electrode of the present invention has at least 10%, preferably at least 20% length of the top region of the protruding shape (such as nanopillars, nanowires or nanoribbons) covered by antibiofouling coating such as PTFE or PEG, with adjacent elongated features (e.g., nanopillar type geometry) still remain separated, so that the bottom part of the elongated features continue to participate in electrical conduction during pulsing or sensing. The antibiofouling coating desirably covers less than 50% of the length of the elongated features so that the electrical conduction sacrifice is not excessive.

(2) Nanopillar or nanoribbon tip coating with anti-biofouling polymer—Anti-biofouling coating can be applied onto local regions of nanostructure top surface by dip coating or contact-print-coating type methods as illustrated in FIG. 18.

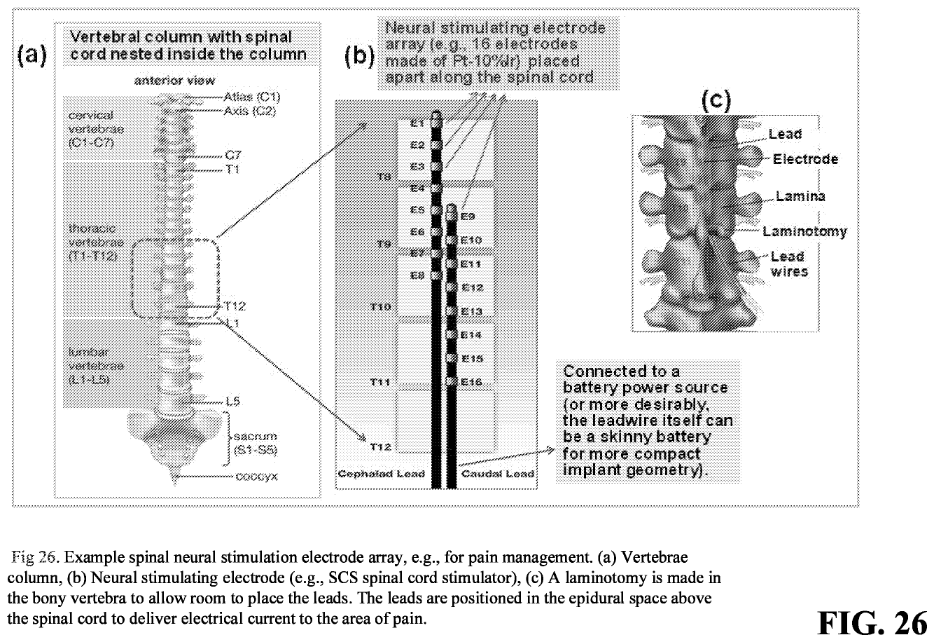

Low impedance, and/or anti-biofouling neural stimulation electrode in the epidural space—Shown in FIG. 26 are schematics of an example spinal neural stimulation electrode array, according to the invention, shown together with bony vertebra structures. These spinal neural stimulation electrode array is e.g., for pain management. Vertebrae column is described in FIG. 26(a), with an example neural stimulating electrode lead (e.g., SCS spinal cord stimulator array with spaced-apart individual electrodes) is shown in FIG. 26(b). A laminotomy is made in the bony vertebra to allow room to place the leads. The lead (with a series of electrodes) is then inserted into the epidural space above the spinal cord and positioned (FIG. 26(c)) to deliver electrical current to the area of pain as needed.

[N]. Reduced Battery Size by Lowered Impedance and/or Skinny Battery Shape

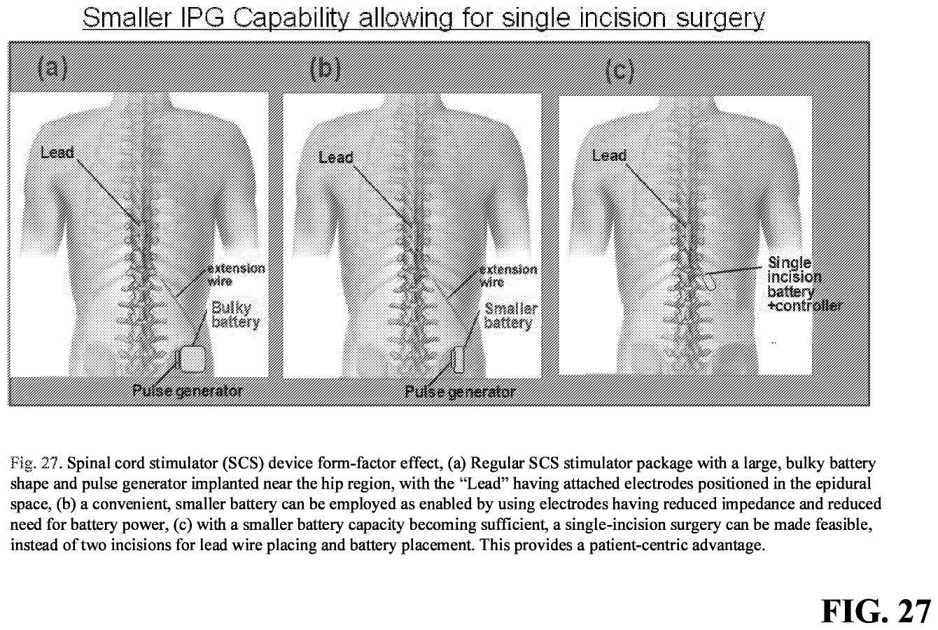

The size and efficiency of power source, e.g., implanted battery pack for spinal cord stimulation (SCS) are important parameters that dictate the useful lifetime of the implanted electrical stimulation package. Shown in FIG. 27 is a schematic illustration of the spinal cord stimulator device form-factor effect. Illustrated in FIG. 27(a) is the regular SCS stimulator package with a large, bulky battery shape (e.g., Li ion rechargeable battery) and pulse generator control circuit implanted near the hip region, with the elongated device “Lead” having attached electrodes positioned in the epidural space. As shown in FIG. 27(b), a convenient, smaller battery can be employed as enabled by using electrodes having reduced impedance and reduced need for battery power. If the impedance of the electrode is reduced by a factor of 5 (as is possible by utilizing a metallic nanopillar structure on the electrode surface, according to the invention), the battery power usage is presumed to be ˜⅕ of the regular electrode, which means that the implanted battery size can be reduced by a factor of 5 to perform with a similar efficiency as the regular SCS stimulator package. Such a drastic reduction of implanted battery size will be advantageous for patients' comfort and maintenance of safe operation of SCS devices and systems.

Shown in FIG. 27(c) is an alternative scheme of dramatically reducing the battery size, for example, by altering the form factor of the battery into size-reduced and elongated, or rod-shape battery which can simultaneously be a part of the lead wires (extension wires) for more compact implanting geometry (multiple rod batteries can be connected in-series for higher voltage or in-parallel for higher current), taking a much smaller space inside a human body. If desired, such a smaller battery can allow a “single-incision” surgery for implanting of spinal stimulation electrode system at the same time as installing of the battery pack, instead of currently implemented, two-incision process of IPG (implantable pulse generator) implanting incision and battery pack placement incision, thus providing a patient-centric advantage of reduced inconvenience to the patient going to the SCS procedures.

Yet another alternative is to have a remote rechargeable system (not shown) by which the implanted battery power is restored once in a while via remote recharging, such as by using a transformer or RF operation of supplying electrical energy for charging of the implanted battery across human body skin.

### [N]. Feedback-Controlled Electrical Stimulatuion of Spinal Cord, Brain and Other Neural Prosthesis

According to the invention, the anti-biofouling, low-impedance electrodes described in the present invention are useful for various neural stimulation or neural sensing devices including cochlear implants for hearing impaired patients, brain neural stimulator implants for patients with epilepsy, Parkinson's Disease, pace maker electrodes, and other neural therapeutics and measurement/monitoring devices.

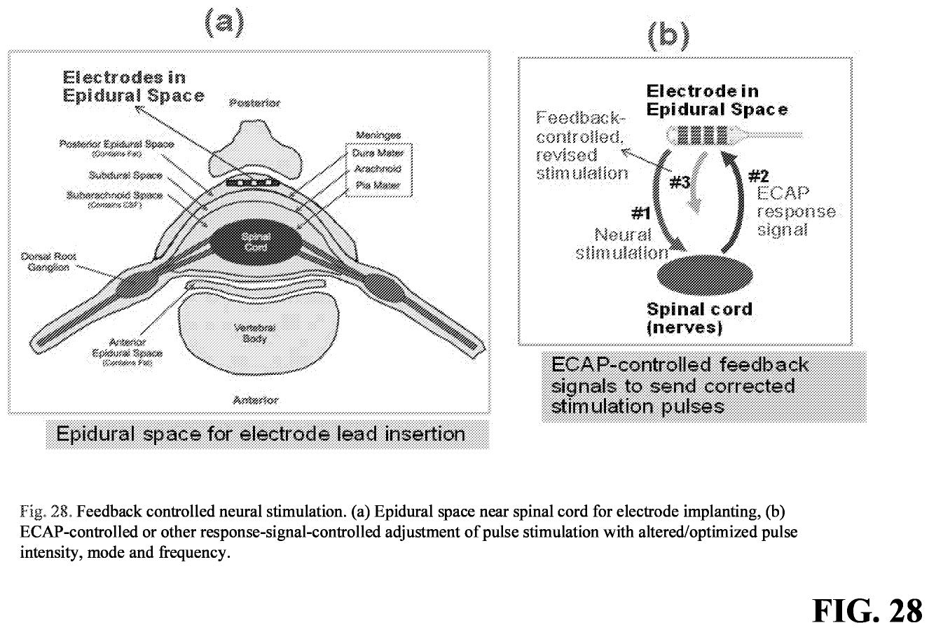

Illustrated in FIG. 28 are schematics associated with feedback controlled neural stimulation, with FIG. 28(a) describing the epidural space near the spinal cord for electrode implanting, FIG. 28(b) describing ECAP-controlled or other response-signal-controlled adjustment of pulse stimulation with altered/optimized pulse intensity, mode and frequency, so that more optimized pulse signals with corrected stimulation intensity or mode can then be supplied. Electrically evoked compound action potential (ECAP) is a human body response signal as a result of each stimulating electrical pulse applied to spinal cord, beep brain or cochlear neural elements. For example, during neural stimulation, transmembrane currents are generated to create recordable voltages near the electrode, and accurate sensing of these ECAP signals is an important aspect to enable feedback-controlled, adjusted pulsing. A reliable and accurate measurement of ECAP signals with high signal-to-noise ratio is therefore important for dependable feedback-controlled neural stimulations. Lowered electrode impedance as disclosed in this invention reduces weakening or partial loss of electrical signal intensity and resolution.

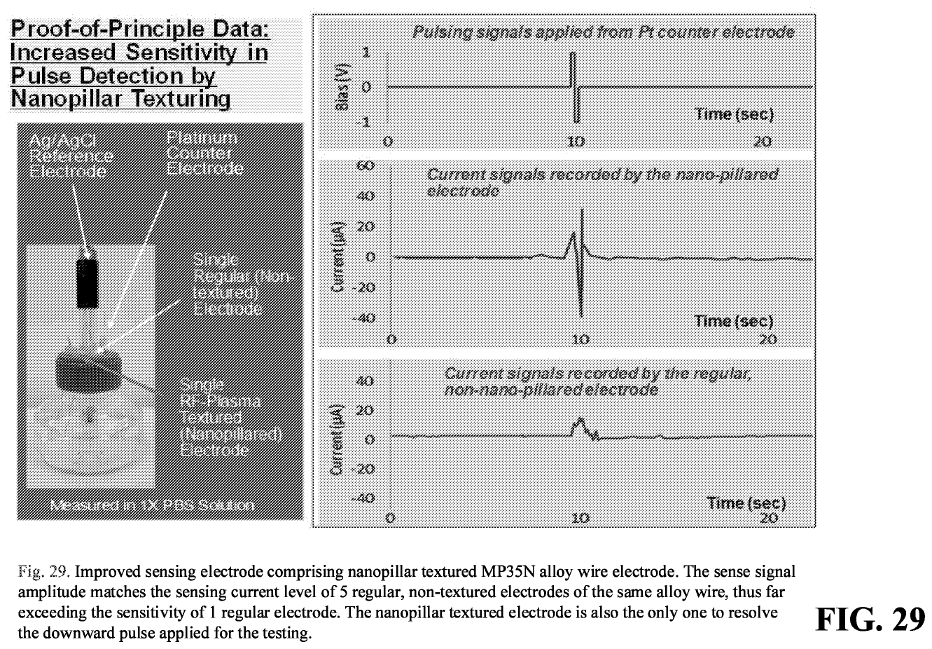

Shown in FIG. 29 is an example experimental data demonstrating the behavior of improved sensing electrode comprising nanopillar textured MP35N alloy wire material, having ˜200 nm diameter and ˜1 um tall nanopillars. RF plasma texturing is performed with the power of 50-500 watt (preferably 100-300 watt) for a duration of 1-60 minutes, preferably 5-20 minutes. RF, microwave or ICP plasma can be utilized for nanotexturing to produce nanopillar or related structures.

The desired nanopillar (or nanopillar-like protruding structure) dimension in the sensing electrode (e.g., made of MP35N type alloy, Pt, Pt—Ir, other novel metal based alloys, stainless steel based alloys or other electrode materials made of metal, alloy, silicon, ceramic, carbon, carbide, nitride, composite and other electrode materials, with optional coating of biocompatible and conducting film coated on the surface) for improved sensing signal is typically in the range of 10-500 nm average diameter, preferably 20-300 nm diameter, and 0.3-100 um height, preferably 1-20 um height. In FIG. 29(a), a train of pulse signal is applied from the Pt counter electrode to the MP35N sensing electrodes. The sense signal amplitude from the nanopillar textured electrode, FIG. 29(b), is significantly higher, matching that of five regular, non-textured electrodes of the same alloy wire FIG. 29(c), thus far exceeding and outperforming the sensitivity of 1 regular, non-textured (smooth surfaced) MP35N electrode of identical dimension. The nanopillar textured electrode is also the only one to resolve the downward pulse shape applied for the sensing tests as the regular electrode failed to detect the downward pulse segment.

The nanotextured electrode alloy such as MP35N or Pt—Ir alloy modified by plasma treatment, thermal treatment, chemical treatment, electrochemical processing for control of elongated nanostructures by additive or subtractive processes provides much enhanced sensing signal, as experimentally demonstrated in FIG. 29. The nanostructured stimulation electrode of the present invention desirably provides at least 50% increased sense signal (in peak current amplitude), preferably at least 100% increased signals, more preferably at least 200% increased signals as compared to the identical sized electrode material with non-textured smooth surface.

### [O]. On-Chip Neural Stimulators and Sensors

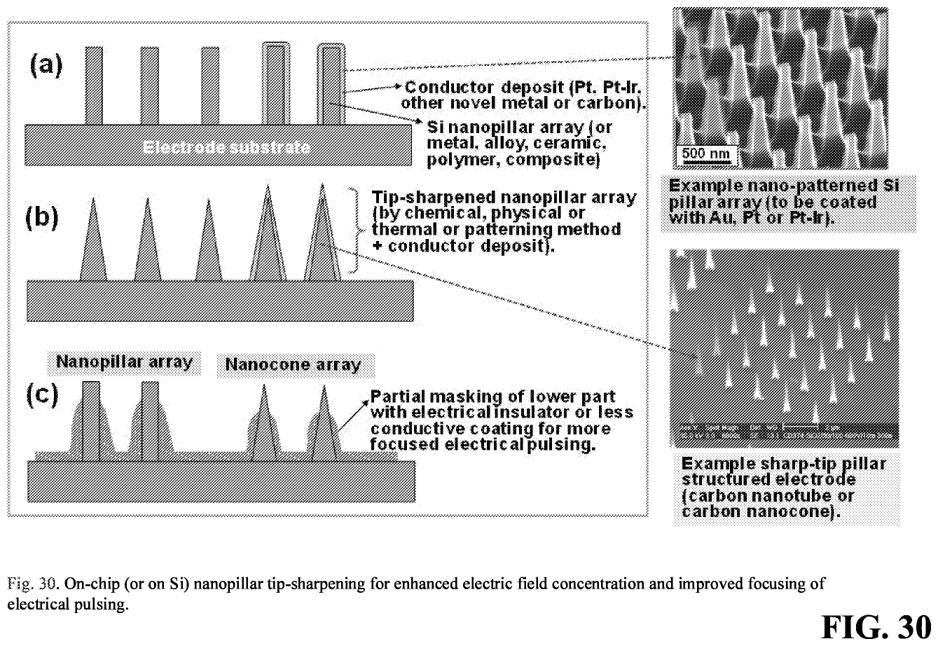

Shown in FIG. 30 are some schematics of on-chip (or on Si) nanopillar array of Si type electrodes (e.g., shaped by lithographical patterning of DUV patterned Si, by additional steps of repeated oxidation and chemical etching of surface SiO₂) or carbon nanotube type electrodes, with an optional (but desirable) coating of conductors (e.g., 3-20 nm thick adhesion layer of Cr, Ti, Zr etc+e.g., 20-200 nm thick Pt, Pt—Ir, Au or other high electrical conductivity electrode metals or alloys or stable and biocompatible carbon or graphite based coating. The coating of conductive metal coating can be achieved by sputter deposition, evaporation deposition, ALD (atomic layer deposition) or other CVD methods, electroless coating or electrodeposition on patterned Si substrate electrodes. These micrometer or sub-micrometer dimension electrodes exhibit lowered impedance values which will result in higher signal sensing resolution, and much lower impedance than Utah or Michigan Micro-electrode array. On-chip nanopillar array electrodes are useful when recording of neural signals are pursued, as the collected electrical signals (such as ECAP or other neural signals) can be processed using implanted circuitry and computerized processor functions within the Si based or other semiconductor-based chips, such that a miniaturization of circuitry can be enabled. The collected electrical signals can alternatively sent to the central process unit, e.g., near the battery type power source, or can be wirelessly transmitted to the processor unit outside human body, and the feed-back controlled electrical pulsing command can be sent again wirelessly to the controller implanted within human body.

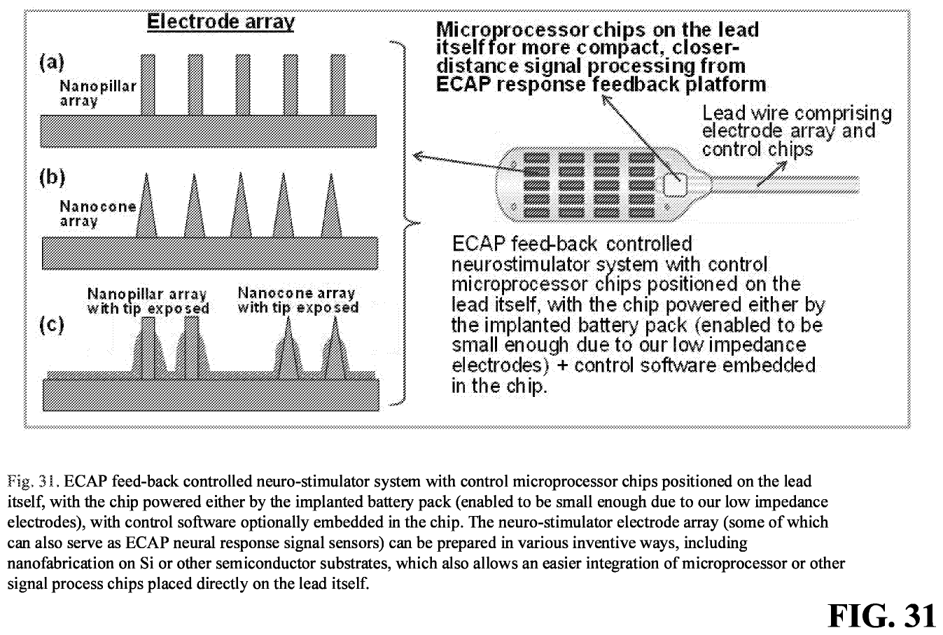

Nanopillar shape alteration can be done by nanopatterning and follow-up processing, (a) Nanopillar array formed by lithography, CVD deposition or other patterning /formation methods. (b) Tip-sharpened nanopillar array (e.g., by selective RIE etching or chemical etching, or by plasma deposition of metallic, ceramic or carbon electrodes), (c) Partially insulating barrier cover material (e.g., polymer or ceramic such as SiO₂, ZrO₂, etc) for more directed, more focused electrical signal pulsing from nanopillars or nanocones. These configurations are illustrated in FIG. 31.

### [P]. Battery Size Reduction

For implantable pulse generator (IPG) devices to be implanted inside human body, a surgery to open up the skin tissue is necessary. Typical spinal cord stimulators package includes electrode lead wires comprising an array of multiple electrodes and a battery pack to supply electrical energy for providing the desired pulse signals. The battery pack also incorporates some control circuits for pulsing. Electrode impedance is one area where changes occurring at the electrode-tissue interface affect power usage. Electrode impedance can be described as the resistance to charge exchange between the electrode surface and the electrolyte. Power is directly proportional to electrode impedance, such that increases in electrode impedance result in increases in the device's power requirements. When the impedance of the electrode is reduced, the electrical power requirement for pulse stimulation is also reduced. For example, if the impedance is reduced by a factor or ×5-10, the battery power requirement can be likewise reduced (which may or may not be proportional). This implies that in order to meet the same electrical power requirement, the size of the battery can be reduced by a factor of ,e.g., roughly ×5-10. Such a substantially reduced battery size also means smaller weight, and the miniaturized battery pack can then be inserted into human body by surgery in a much easier way.

### [Q]. Mechanical Protection of Nanostructured Neural Electrodes

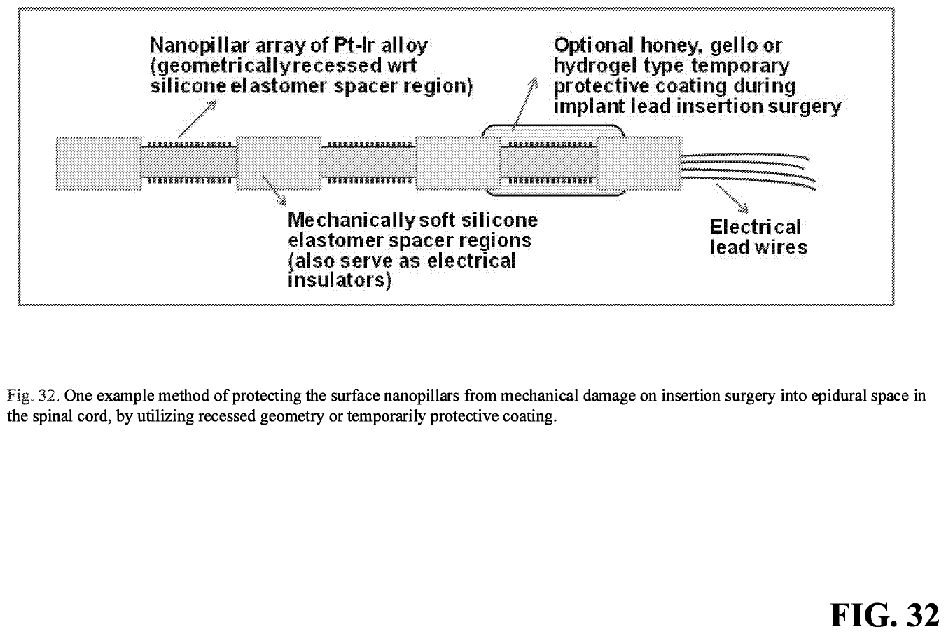

The nanopillar or nanowire configuration on the electrode surface can be damaged during surgery on insertion to the epidural space, if the process is not carefully performed. In order to minimize such mechanical damage (e.g., nanowire falling off or plastic bending), the nanowire surface can be structured so as to be geometrically recessed relative to the plastic or polymer insulating spacer as illustrated in FIG. 32. Another possibility is to utilize a temporarily protective coating such as made of gelatin (a mixture of peptides and proteins produced by partial hydrolysis of collagen from the skin, bones, and connective tissues of animals), starch, jello, syrup, honey, hydrogel and other dissolvable material.

### [R]. Other Power Sources for Neural Stimulator

As the reduction of battery power use is an important factor, which can be realized e.g., by reduced electrical impedance, there are other means of minimizing the power consumption. Some examples include the use of natural power generation using human body itself, such as enzymatic biofuel cell or glucose based biofuel cells for power generation, thermoelectric power generation utilizing temperature gradient or temperature difference between different parts of human body, or use of body motion (e.g., walking) utilizing piezoelectric generator or electromagnetic power generation (e.g., walking motion inducing movement of magnetic component near solenoid array).

### [S]. Manufacturing Methods for Scale-Up Applications

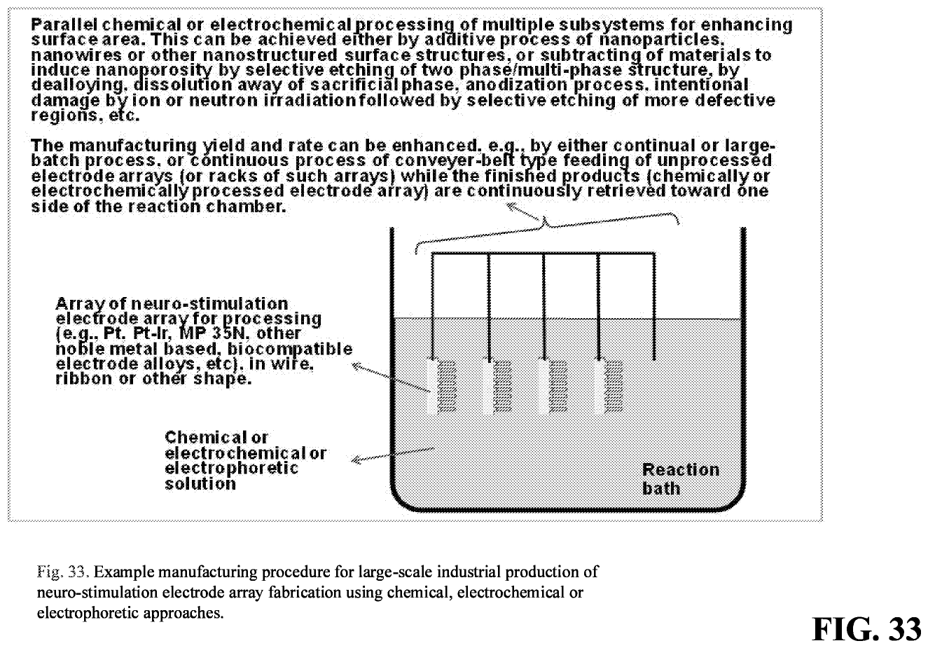

For scale up manufacturing of advanced, large-surface-area neural electrodes, the scale and speed of electrode processing is one of the important parameters. According to the invention, some new manufacturing process approaches can be utilized as discussed in the following schematics. These are illustrated in FIG. 33 to FIG. 35.

Illustrated at FIG. 33 is an example manufacturing procedure for large-scale industrial production of neuro-stimulation electrode array fabrication using chemical, electrochemical or electrophoretic approaches.

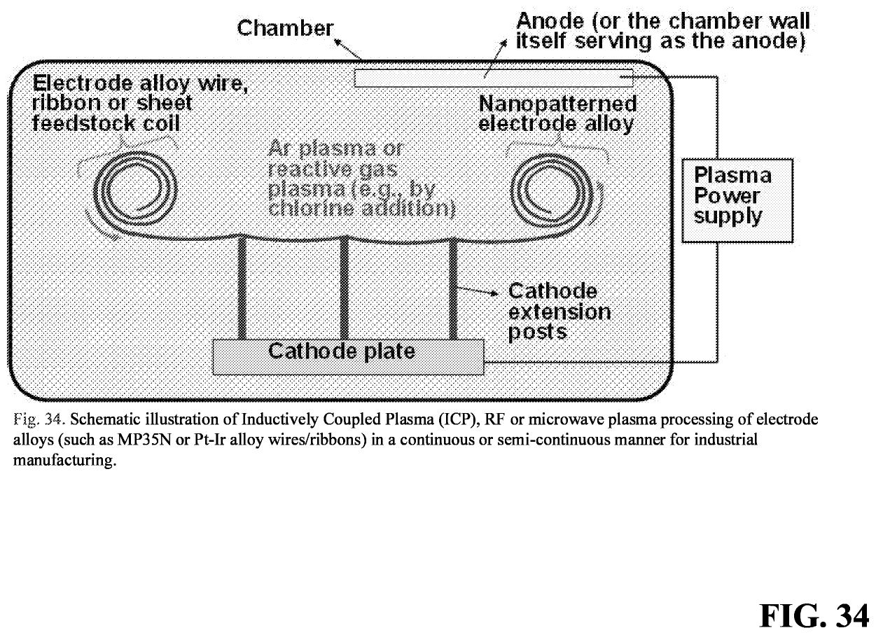

Illustrated at FIG. 34. Schematic illustration of Inductively Coupled Plasma (ICP), RF or microwave plasma processing of electrode alloys (such as MP35N or Pt—Ir alloy wires/ribbons) in a continuous or semi-continuous manner for industrial manufacturing.

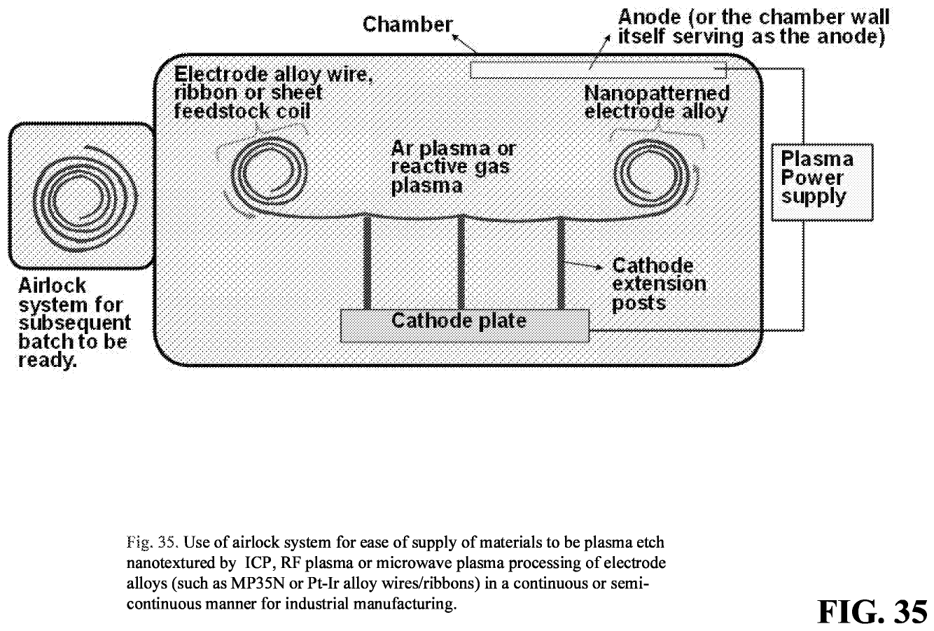

Illustrated at FIG. 35 is the use of airlock system for ease of supply of materials to be plasma etch nanotextured by ICP, RF plasma or microwave plasma processing of electrode alloys (such as IVIP35N or Pt—Ir alloy wires/ribbons) in a continuous or semi-continuous manner for industrial manufacturing.

### [T]. Electrode Geometry Selection.

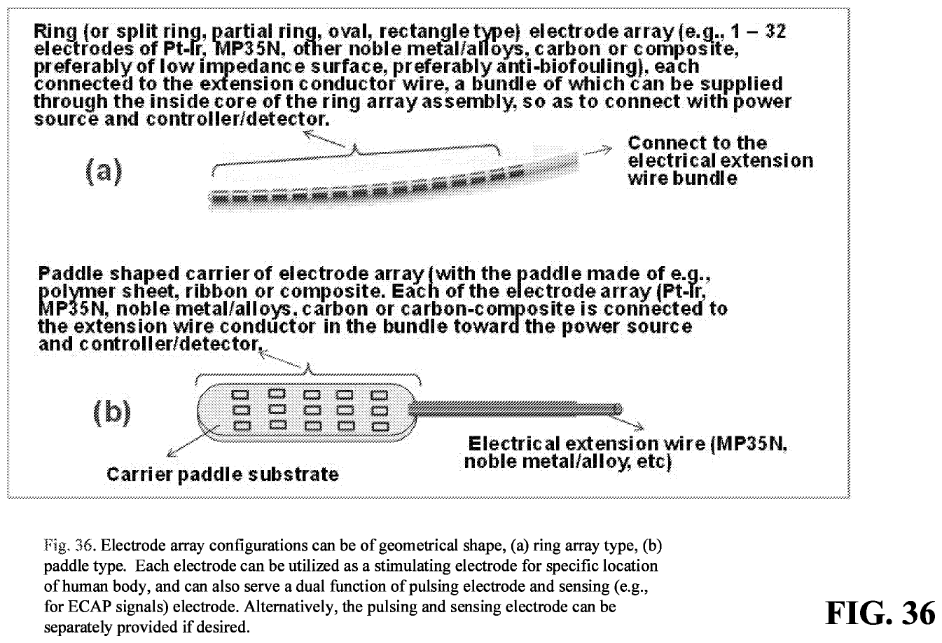

Shown in FIG. 36 are electrode array configurations with different geometrical shape, (a) ring array type, (b) paddle type. Each electrode can be utilized as a stimulating electrode for specific location of human body, and can also serve a dual function of pulsing electrode and sensing (e.g., for ECAP signals) electrode. Alternatively, the pulsing and sensing electrode can be separately provided if desired. The number of electrodes can be in the range of e.g., 1-120, preferably in the range of 4-32 especially for SCS type applications. The electrodes on the paddle can face only one side or can be exposed to both top and bottom sides.

Illustrated at FIG. 36 is an electrode array configurations can be of geometrical shape, (a) ring array type, (b) paddle type. Each electrode can be utilized as a stimulating electrode for specific location of human body, and can also serve a dual function of pulsing electrode and sensing (e.g., for ECAP signals) electrode. Alternatively, the pulsing and sensing electrode can be separately provided if desired.

[U]. Lead Assembly from Ring Electrode Array

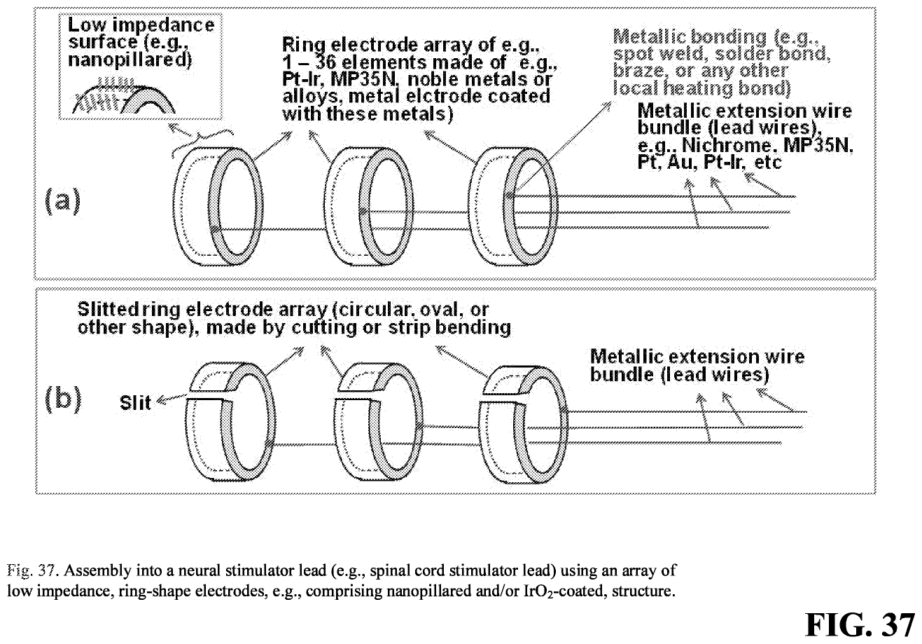

Shown in FIG. 37 is a process of assembling an array of ring electrodes into a neural stimulator lead (e.g., spinal cord stimulator lead) using low impedance, ring-shape electrodes, e.g., comprising nanopillared and/or IrO₂-coated, structure. Each of the electrode ring can have a geometry of circular ring (FIG. 37(a)), slitted ring FIG. 37(b)), or other shapes. The slitted shape ring can be made by either bending of strip electrode or length-cutting of slitted cylinder made by bending of metal strip. Once the electrodes are connected to the extension conductive wire (high electrical resistance wire is desired if high frequency pulsing is to be used), the electrode array can be position-fixed by casting with a heat-curable, UV-curable, or catalyst-curable polymer that can be hardened. Optionally the core of the ring array can be partially filled with a stiffening material (polymer, composite, metal, etc) for ease of handling or insertion into desired neural location. Alternatively for the ease of lead insertion into the epidural space in the spinal cord region, the outside of the lead wire can be stiffened using a high strength polymer, such as polyether ether ketone (PEEK) or ultra-high-molecular-weight polyethylene (UBMWPE), which can be removed together with the guide tube.

Alternatively, a dissolvable polymer such as sucrose, honey, gelatin or other water-soluble polymer can be utilized as a temporary stiffener for ease of lead insertion into the epidural space, which can be naturally dissolved away some time after the insertion surgery.

### [V]. Springy, Gap-Reducing Electrode Structure.

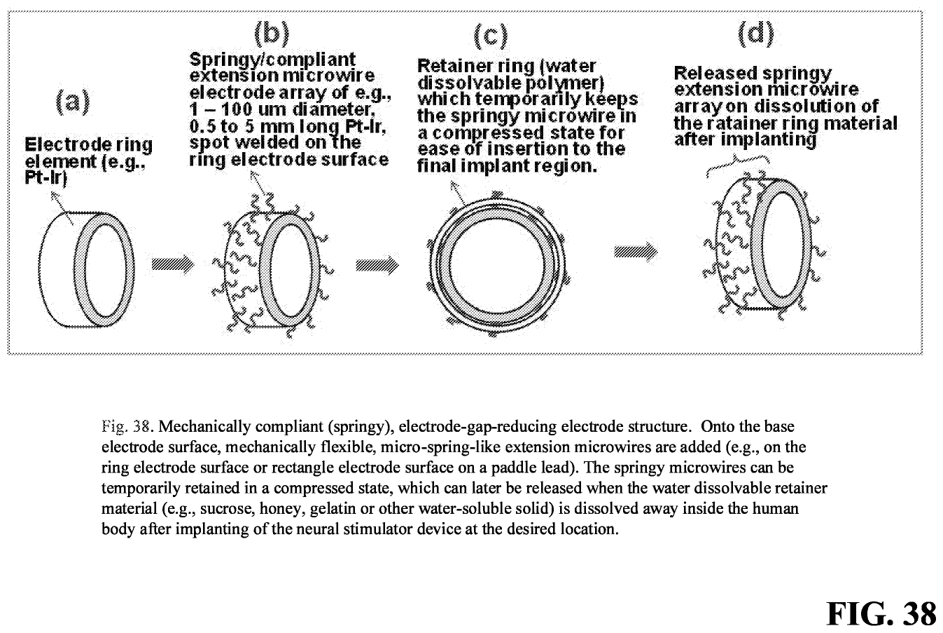

Shown in FIG. 38 is a schematic illustration of mechanically compliant (springy) extension microwire from the base electrode (e.g., off the ring electrode surface or rectangle electrode surface on a paddle lead). These microwire springs can be temporarily retained in a compressed state, which can later be released when the water dissolvable retainer material such as sucrose, honey, gelatin or other water-soluble polymer or compound is dissolved away inside the human body after implanting of the neural stimulator device at the desired location.

The main advantage of such a springy microwire structure is that the springy electrode tip can become closer to (or even directly contact) the tissue or neuro-responsive organ for more powerful electrical pulse amplitude stimulation due to the reduced gap. In reducing the gap between actuating/pulsing electrode tip and the tissue or organ that is to be pulsed, the mechanical compliance is a very important requirement to prevent undesirable poking into the tissue or organ and to ensure safety of the human subject. Instead of the dissolvable solid, the microwire array can also be retained in a compressed state by an alternative configuration of tentative mechanical confinement of pre-outward-stretched (diameter wise) microwire bundle within a guide tube, with the microwire array allowed to be released to be expanded/stretched outward by removing the guide tube once the device is inserted into the desired location of human body.

The spot welding (or laser welding) of microwires can be performed in the FIG. 38(b) stage or FIG. 38(c) stage. The latter could be easier for mass production as the whole ring-shaped microwire assembly can be spot welded to the base ring electrode using a one step process of utilizing a clam-shell type spot welder configuration.

### [W]. Protection of Nanopillar Structure by Shoulder Arrangement

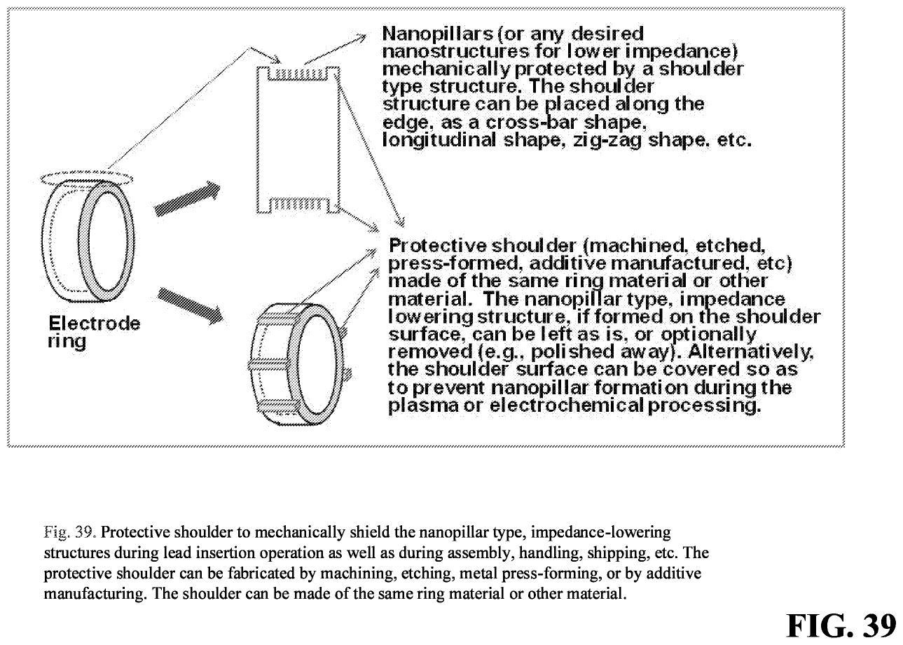

In order to make sure that the nanopillar structure or related nanostrucutres on the electrode surface does not get scrubbed away during the lead insertion operation, a protective shoulder is provided (FIG. 39) to mechanically shield the nanopillar type, impedance-lowering structures during lead insertion, as well as during assembly, handling, shipping, etc. The protective shoulder can be fabricated by various means, such as machining, etching, metal press-forming, or by additive manufacturing.

The shoulder can be made of the same ring material or other material. The nanopillar type, impedance lowering structure, can optionally be removed from the shoulder surface if desired (e.g., by polishing or etching away). Alternatively, the shoulder surface can be masked to prevent nanopillar formation during the plasma or electrochemical processing.

### [X]. Manufacturing Approaches for Ring Electrode Process for Low Impedance

Multiple electrode rings with low impedance as prepared according to the invention have to be put together to construct neurostimulation lead device. Shown in FIG. 39 is an example manufacturing process of ring electrodes (closed ring or split ring) having low impedance surface can be achieved by e.g., (i) plasma surface texturing to form nanopillar surface structure, (ii) chemical etching, (iii) anodization, (iv) electrochemical deposition of radial nanopillars, etc.

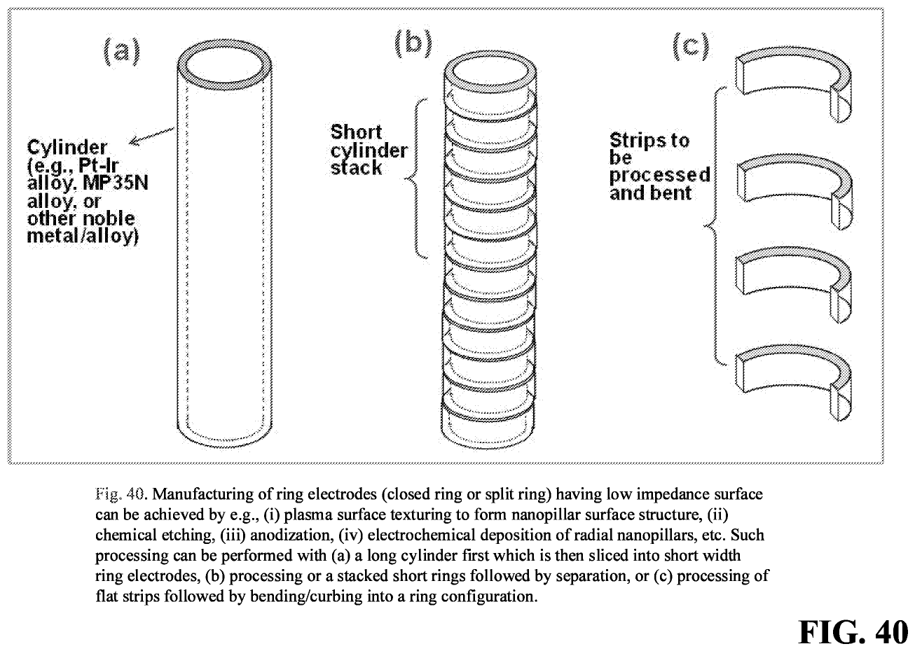

Such surface structure altering processing can be performed with a long cylinder first (FIG. 40(a)), which is then sliced into short width ring electrodes.Alternatively, a stacked short rings can be surface processed ((FIG. 40(b)), followed by separation.Another option is to process flat strips followed by bending/curbing into a ring configuration (FIG. 40(c)). Some shoulder structure can optionally be added near the edge of the strips so that the nanopliiars are not mechanically damaged during bending operation or other mechanical shaping, or during handling.

At FIG. 40 is a manufacturing of ring electrodes (closed ring or split ring) having low impedance surface can be achieved by e.g., (i) plasma surface texturing to form nanopillar surface structure, (ii) chemical etching, (iii) anodization, (iv) electrochemical deposition of radial nanopillars, etc. Such processing can be performed with (a) a long cylinder first which is then sliced into short width ring electrodes, (b) processing or a stacked short rings followed by separation, or (c) processing of flat strips followed by bending/curbing into a ring configuration.

### [Y]. Drug Release Structure Using Nanopillar Type Configuration

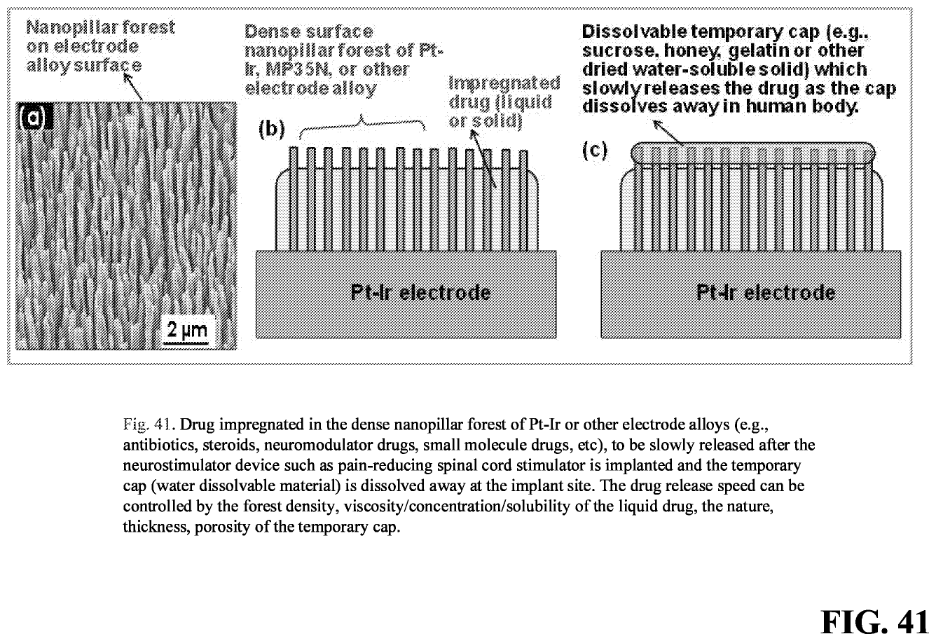

The nanopillar type structure that lowers electrode impedance (FIG. 41(a)) can also be utilized, according to the invention, as a convenient means of slow drug delivery. In spinal cord related surgery, application of some drug is useful, such as antibiotics, steroids, neuromodulator drugs, or small molecule drugs. Shown in FIG. 41(b) is an example schematics of drug impregnation in the dense nanopillar forest of Pt—Ir or other electrode alloys, to be slowly released after the neurostimulator device such as pain-reducing spinal cord stimulator is implanted and the temporary dissolvable cap material (such as sucrose, honey, gelatin or other water-soluble polymer) is dissolved away in human body. The drug release speed can be controlled by the nanopillar forest density, viscosity/concentration/solubility of the drug, the nature, thickness and porosity of the temporary dissolvable cap. A solid shape drug (e.g., by drying off the impregnated liquid drug) can also be utilized.

Illustrated at FIG. 41 is a drug impregnated in the dense nanopillar forest of Pt—Ir or other electrode alloys (e.g., antibiotics, steroids, neuromodulator drugs, small molecule drugs, etc), to be slowly released after the neurostimulator device such as pain-reducing spinal cord stimulator is implanted and the temporary cap (water dissolvable material) is dissolved away at the implant site. The drug release speed can be controlled by the forest density, viscosity/concentration/solubility of the liquid drug, the nature, thickness, porosity of the temporary cap.

### [Z]. Ex-Vivo Simulated Evaluation of Electrode Performance.

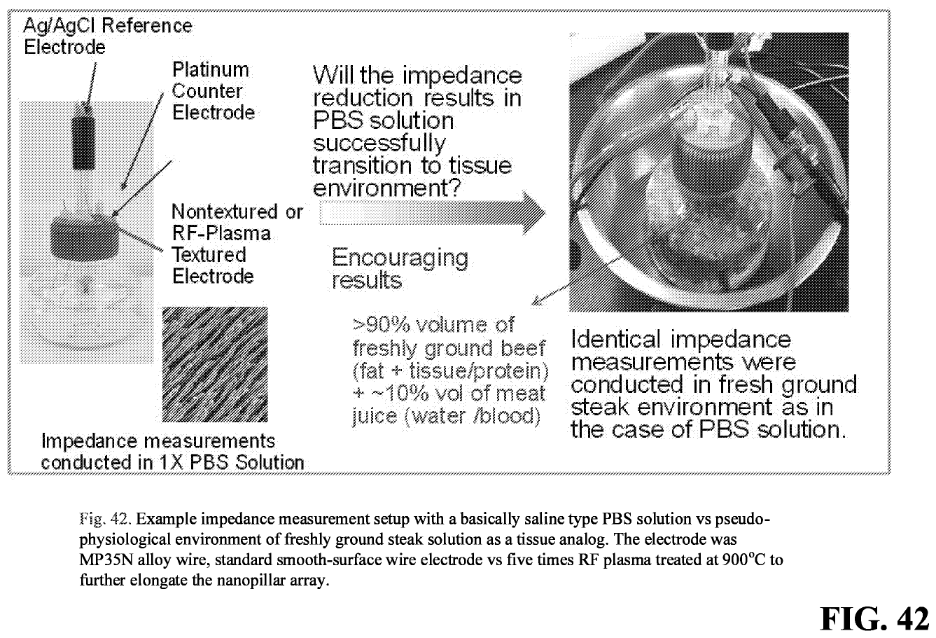

For more accurate evaluation of electrode performance in real animal or human body, simple PBS solution environment may not be truly accurate. Therefore, a simulated ex-vivo environment is useful for more reliable evaluation of electrode performance as described in FIG. 42 to FIG. 44.

Illustrated at FIG. 42 is an example impedance measurement setup with a basically saline type PBS solution vs pseudo-physiological environment of freshly ground steak solution as a tissue analog. The electrode was MP35N alloy wire, standard smooth-surface wire electrode vs five times RF plasma treated at 900° C. to further elongate the nanopillar array.

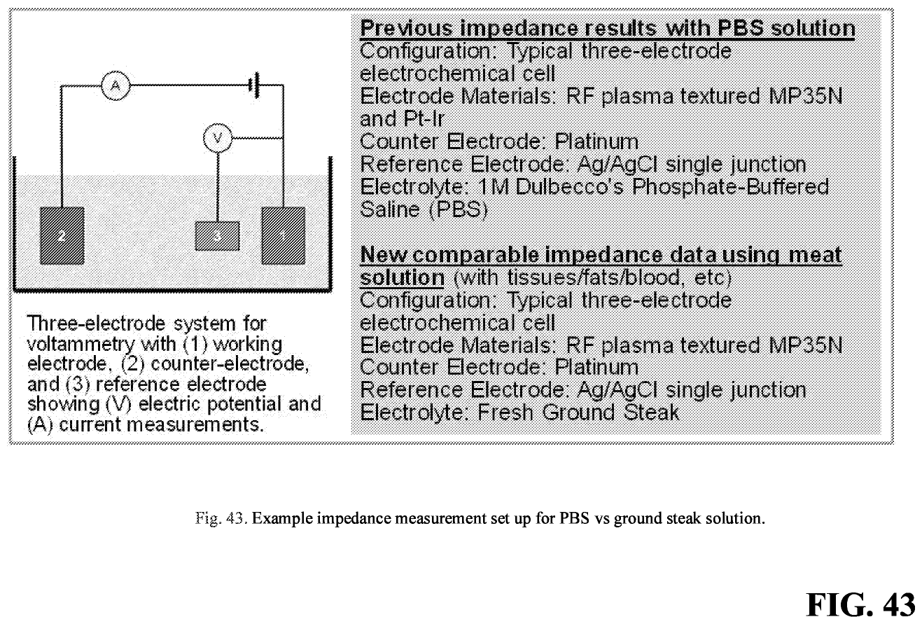

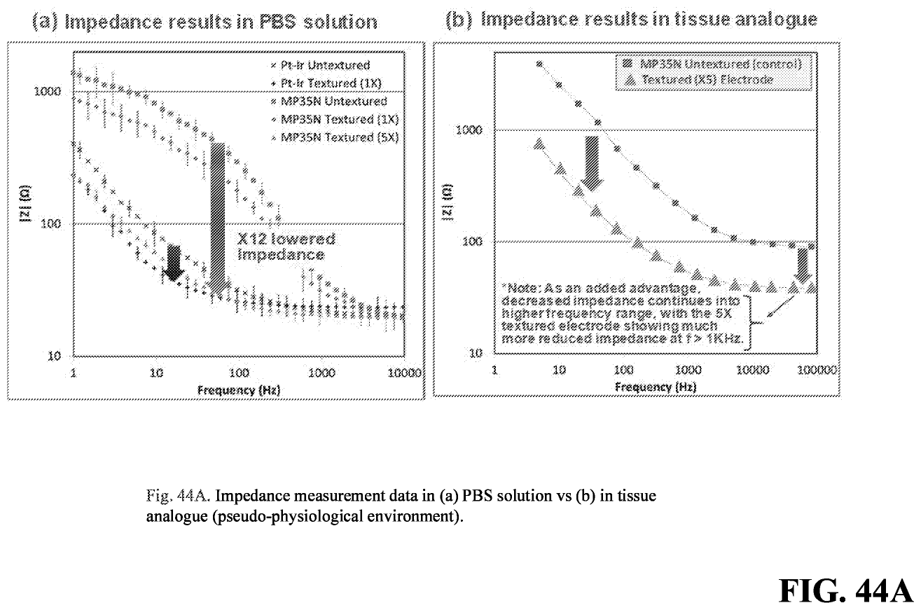

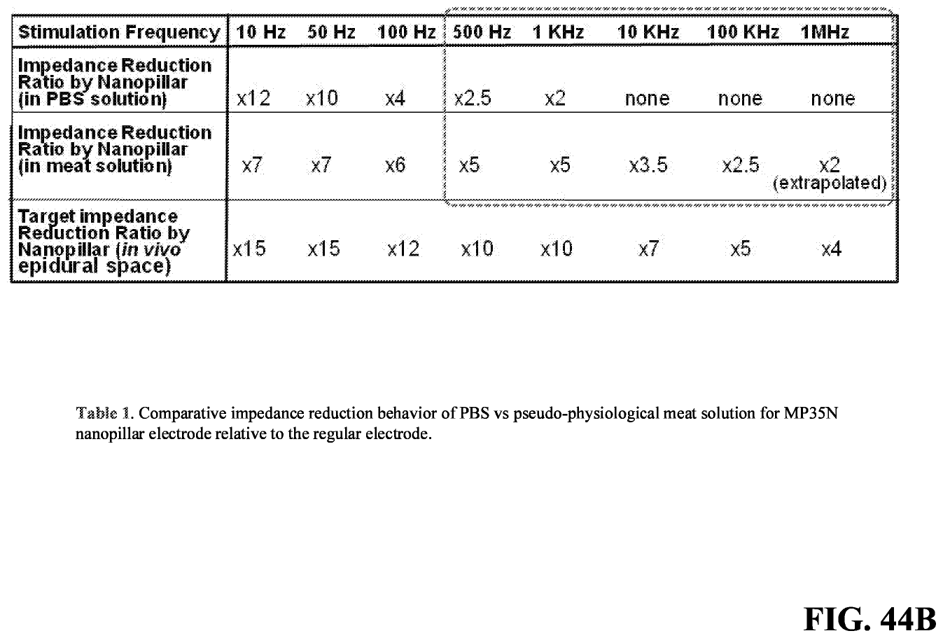

Illustrated at FIG. 43 is an example impedance measurement set up for PBS vs ground steak solution. Illustrated at FIG. 44 is an impedance measurement data in (a) PBS solution vs (b) in tissue analogue (pseudo-physiological environment). Illustrated at Table 1 is a comparative impedance reduction behavior of PBS vs pseudo-physiological meat solution for MP35N nanopillar electrode relative to the regular electrode.

In simulated physiological environment (freshly ground steak meat, 90% volume solution), the impedance reduction by nanopillar electrodes is still maintained. For high frequency stimulation, the meat solution environment actually increases the impedance reduction. At 1 KHz or higher, the meat solution makes the nanopillar electrode even more attractive than the regular electrode. For higher frequency of 100 KHz to 1 MHz, the PBS solution does not make the nanopillar electrode any better than the regular electrode, but the pseudo-physiological environment makes the nanopillar electrode superior to regular electrode (which shows no improvement in impedance reduction). With a possibility to make the nanopillar taller, e.g., by a factor of two with additional cycles of RF plasma processing, a further reduction in impedance is anticipated and targeted as shown in Table 1.

### [AA]. Chemical or Electrochemical Pre-Etch Treatment on Electrode Surface

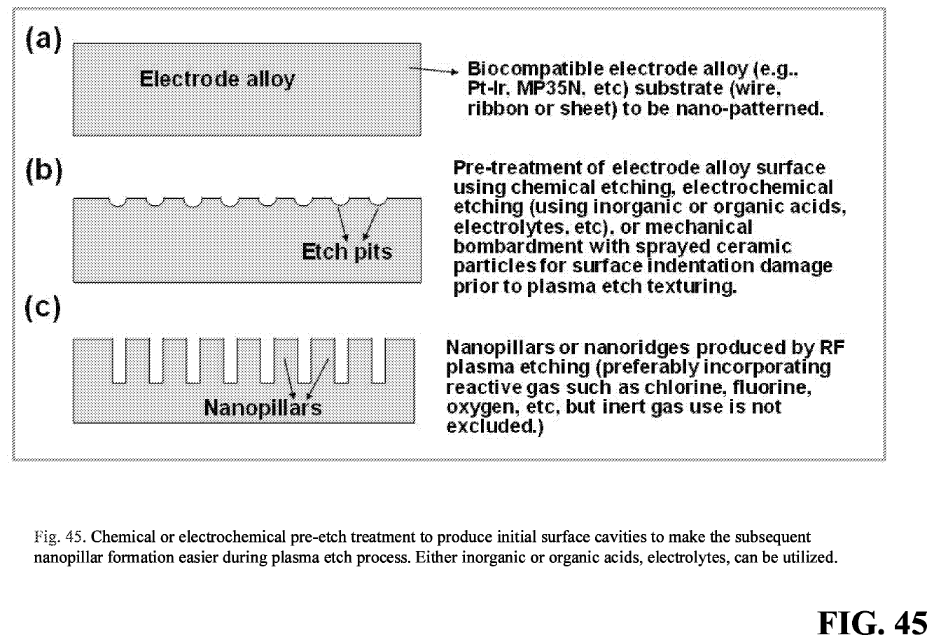

The surface nanotexturing using plasma etching, either using a reactive gas (e.g., comprising chlorine, fluorine, bromine, oxygen, hydrogen) or inert/semi-inert gas (such as Ar, He), can be improved if a chemical or electrochemical pre-etch treatment is provided onto the electrode surface, as described in FIG. 45. Such a pre-etch treatment produces initial surface cavities (inhomogenieties) to make the subsequent nanopillar formation easier during plasma etch process. Either inorganic or organic acids or electrolytes can be utilized for chemical pitting pre-treatment.

An alternative method of introducing etch pit seeds for local activation of plasma etching is to employ mechanical bombardment with sprayed ceramic particles (such as alumina, titania, diamond nanoparticles, or other hard material micro- or nano-particles) for surface indentation damage prior to plasma etch texturing. A beneficial side effect is that some of the particles might get embedded into the electrode alloy surface, in which case, the particle could serve as a mask particle for desirably non-uniform plasma etching.

For the subsequent plasma etching process of nanopillar or nanostructure formation, various plasma etch process can be employed, e.g., RF plasma, microwave plasma, DC plasma, etc, preferably incorporating a reactive gas (at least partially) such as chlorine, fluorine, oxygen, etc. The possibility of using a fully insert gas atmosphere (such as Ar, He, N2) is not excluded.

### [BB]. Pre-Depositing of Less-Plasma-Etchable Nanoscale Masks

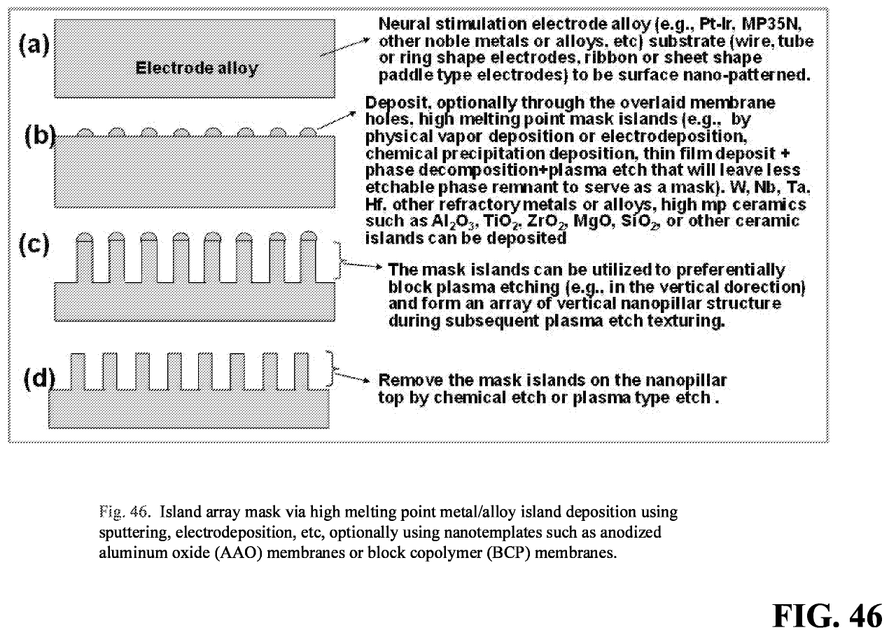

Yet another means of introducing inhomehenieties in plasma etching is to pre-deposit masking nanostructures on the electrode surface desirably in the form of nano islands or nano features in general, prior to the plasma etch texturing, as illustrated in FIG. 46. Such deposition can be carried out by sputtering or e-beam evaporation (of e.g., high melting point metals such as W, Nb, Ta, Hf, other refractory metals and alloys that tend to plasma etch less, high mp ceramics such as Al₂O₃, TiO₂, MgO, SiO₂, refractory metal oxide, rare earth oxide, nitrides, carbides, or mixed ceramics, etc that tend to resist plasma etch). Alternatively, electrodeposition or cold spray deposition can be used to deposit high mp metal islands (such as W, Nb, Ta, Hf, other refractory metals or alloys, oxides, nitrides, carbides, fluorides) onto electrode surface.

Optionally nanopatterning by AAO (anodized aluminum oxide membranes, diblock copolymer membranes, or by lithography means (for flat substrates) can also be utilized to provide higher mp nanoscale caps on the electrode alloy surface, which is then followed by plasma etching process to form nanopillars utilizing the masking cap.

Illustrated at FIG. 46 is an island array mask via high melting point metal/alloy island deposition using sputtering, electrodeposition, etc, optionally using nanotemplates such as anodized aluminum oxide (AAO) membranes or block copolymer (BCP) membranes.

### [CC]. Previously Plasma Textured Surface Almost Removed for Next Plasma Texturing Seed Formation

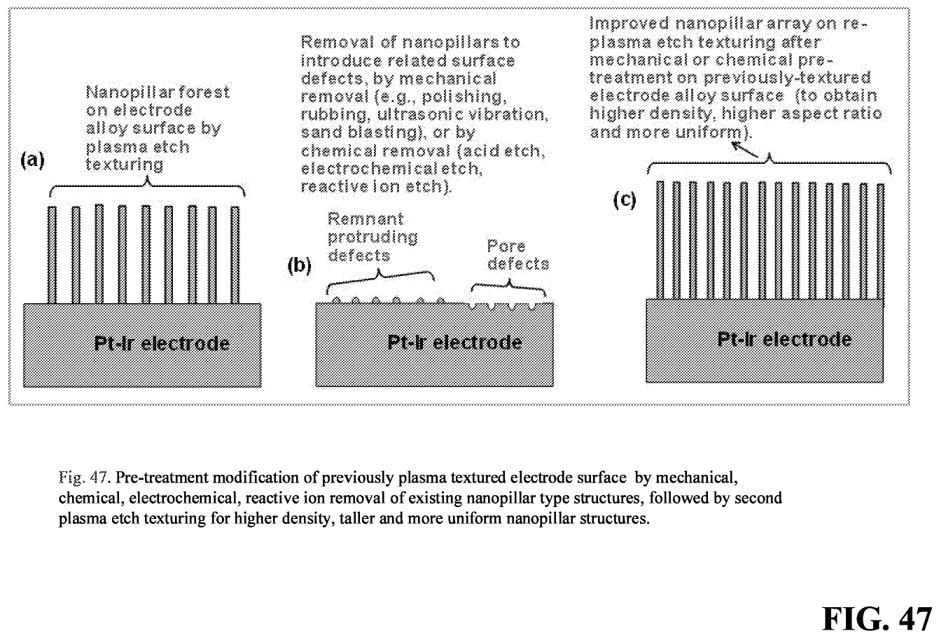

Another alternative method of introducing defective or strained electrode surface is to perform a pre-treatment modification of previously plasma textured electrode surface by mechanical, chemical, electrochemical, reactive ion removal of existing nanopillar type structures, followed by second plasma etch texturing for higher density, taller and more uniform nanopillar type structures (or nanostructures in general). This is schematically illustrated in FIG. 47.

Various techniques can be utilized for removal of existing nanopillars and associated materials from electrode surface, such as mechanical removal (e.g., polishing, rubbing, ultrasonic vibration, sand blasting), or by chemical removal (acid etch, electrochemical etch, reactive ion etch). These mechanical or chemical pre-treatments introduce surface defects such as protruding defects, recessed pores, elastically or plastically stressed regions, etc so as to make nucleation of nanostructure formation easier during the second plasma etch texturing toward a higher density and taller nanotexturing.

This process (nanopillar type formation+removal) can be repeated multiple times (e.g., 2-10 times) in order to gradually improve the density of the nanopillar or related structures on the electrode surface.

Illustrated at FIG. 47 is a pre-treatment modification of previously plasma textured electrode surface by mechanical, chemical, electrochemical, reactive ion removal of existing nanopillar type structures, followed by second plasma etch texturing for higher density, taller and more uniform nanopillar structures.

### [DD]. Pre-Place a Nano Membrane/Mask for Subtractive, Selective Local Surface Pitting, Through the Membrane Openings

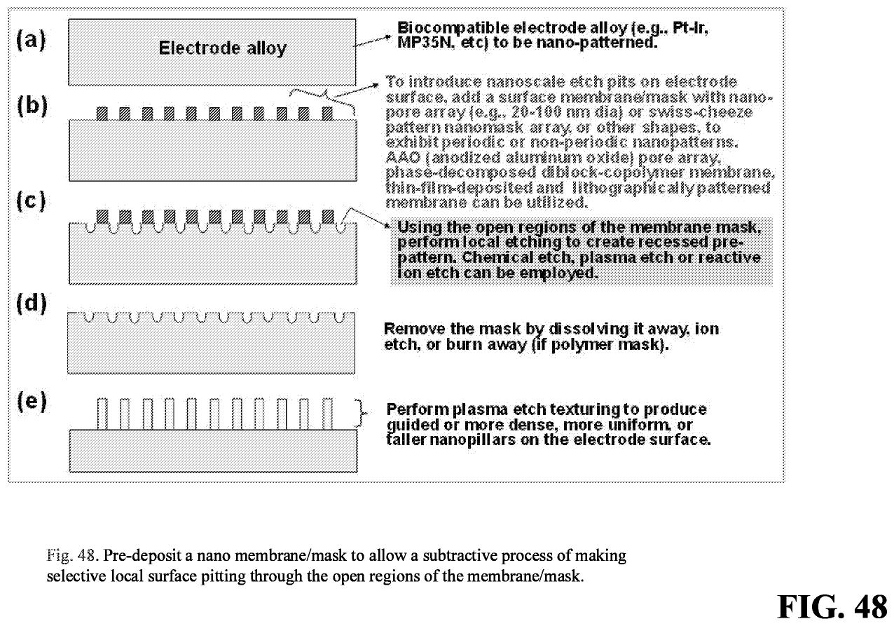

Another way of providing a plasma-etch-starting preform on electrode surface is to pre-place a nano membrane/mask to allow selective local surface pitting through the open regions of the membrane/mask, as described in FIG. 48.

In this approach, the surface is pre-patterned with a periodic or non-periodic membrane/mask with nano-pore array (e.g., 20-100 nm dia) or swiss-cheeze pattern nanomask array, or other shapes. The membrane can have an aspect ratio of e.g., 2-10. Various techniques can be utilized for this approach, such as aluminum sputter deposition and anodizing to form AAO (anodized aluminum oxide) membrane pore array, scoop-up placement of pre-made AAO membranes floating on water, alcohol or other liquids, formation of nanohole array block-copolymer membrane (diblock or triblock-copolymer) by depositing a thin film of e.g., PMMA-polystyrene diblock copolymer and decomposing or scoop-up placement of floating membrane from liquid, lithographically patterned membrane and related methods can be used.

### [EE]. Additive, Selective Local Surface Protrusions Through the Membrane Openings for Improved Plasma Texturing

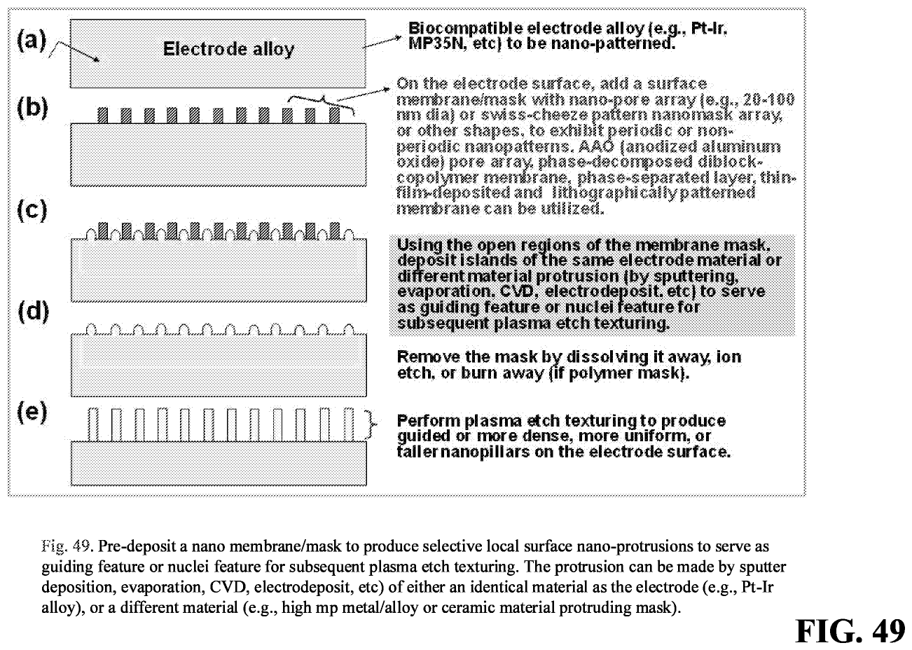

Yet another method to make the plasma etch more controllable is to pre-place a nano membrane/mask on electrode surface to produce selective local surface nano-protrusions (nanobumps) to serve as guiding feature or nuclei feature for subsequent plasma etch texturing, as shown in FIG. 49. The protrusion can be made by material deposition through the nanopores in the membrane, e.g., by sputter deposition, evaporation, CVD, electrodeposit, etc) of either an identical material as the electrode (e.g., Pt—Ir alloy nanobumps on Pt—Ir alloy electrode surface), or a different material.

The use of different material as the nanobumps, especially higher melting point, metal/alloy or ceramic material have certain advantages as these nanobumps can serve as nanomask islands during plasma etch texturing to assist in producing finer, well defined nanopillar array. The materials for the nanobumps deposited through the membrane pores can be selected from e.g., W, Nb, Ta, Hf, other refractory metals and alloys that tend to plasma etch less, high mp ceramics such as Al₂O₃, TiO₂, MgO, SiO₂, refractory metal oxide, rare earth oxide, nitrides, carbides, or mixed ceramics, etc that tend to resist plasma etch. Alternatively, electrodeposition or cold spray deposition can also be used to deposit high mp nanobump islands onto the electrode surface.

The surface membrane/mask can have a nano-pore array (e.g., 20-100 nm dia) or other shapes. The membrane can have an aspect ratio of e.g., 2-10. Various techniques such as AAO (anodized aluminum oxide) pore array, phase-decomposed diblock-copolymer membrane, lithographically patterned membrane and related methods can be utilized.

Illustrated at FIG. 49 is a pre-deposit a nano membrane/mask to produce selective local surface nano-protrusions to serve as guiding feature or nuclei feature for subsequent plasma etch texturing. The protrusion can be made by sputter deposition, evaporation, CVD, electrodeposit, etc) of either an identical material as the electrode (e.g., Pt—Ir alloy), or a different material (e.g., high mp metal/alloy or ceramic material protruding mask).

### [FF]. Plastic and Elastic Deformation of Nanopillar Type Structures and Re-Plasma Etch Texturing

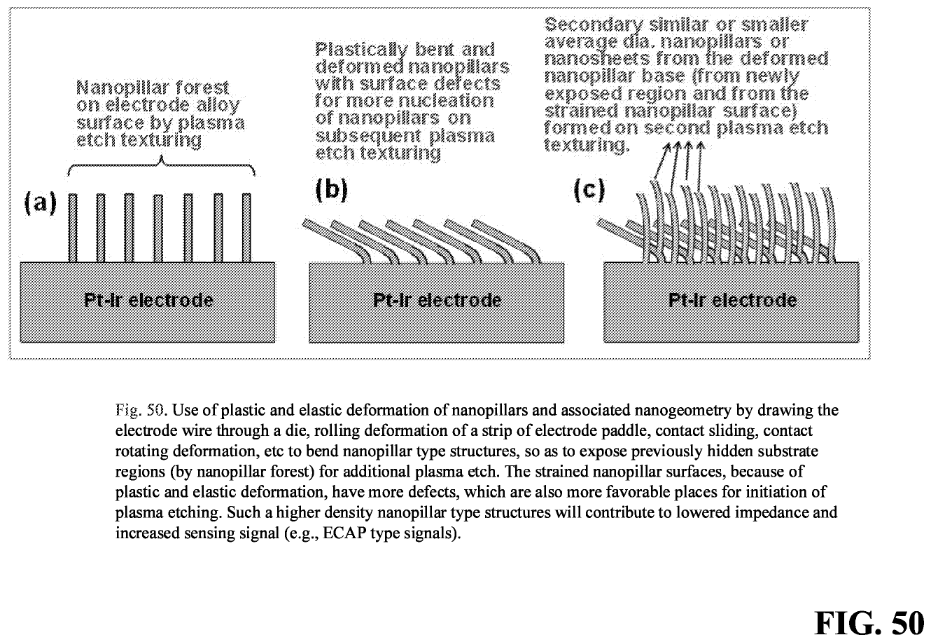

Yet another method of introducing more defects and inhomogenieties for finer scale, enhanced plasma etch texturing is to incorporate plastic and elastic deformation of nanopillars and associated nanogeometry, see FIG. 50, by drawing the electrode wire through a die, rolling a strip of electrode paddle, contact sliding, contact rotating deformation, etc to bend nanopillar type structures, so as to expose previously hidden substrate regions (by nanopillar forest) as well as the side surface of nanopillars, for additional plasma etch and nanostructure development. The strained nanopillar surfaces, due to the plastic and elastic deformation, have more defects, which are also more favorable places for initiation of plasma etching. As discussed in the specification, higher density nanopillar type structures exhibit increased electrode surface area and contribute to lowered impedance as well as to increased sensing signals including ECAP (evoked compound action potential) type signals.

Illustrated at FIG. 50 is a use of plastic and elastic deformation of nanopillars and associated nanogeometry by drawing the electrode wire through a die, rolling deformation of a strip of electrode paddle, contact sliding, contact rotating deformation, etc to bend nanopillar type structures, so as to expose previously hidden substrate regions (by nanopillar forest) for additional plasma etch. The strained nanopillar surfaces, because of plastic and elastic deformation, have more defects, which are also more favorable places for initiation of plasma etching. Such a higher density nanopillar type structures will contribute to lowered impedance and increased sensing signal (e.g., ECAP type signals).

### [GG]. Prevention of Plasma Texturing and Nanopillar Formation on Selected Regions of SCS Stimulation Electrodes.

In SCS leads comprising an array of ring electrodes, some portion of the electrode surface area needs to be free of nanopillar or other nanowires (e.g., ring electrode cross-sectional surfaces and ring-inside-surfaces), so as to prevent inadvertent falling off of metallic nanopillars and resultant loose metallic nano pillars or nano whiskers that might cause electrical shorting or induce nanotoxicity-type health hazards of sharp nanofibers. The presence of such nanopillar type whiskers on the electrode ring side surface or ring-inside-surfaces may also interfere with spot welding connection of extension conductor wires. These problems could occur on both complete-ring shape electrodes or split-ring shape electrodes.

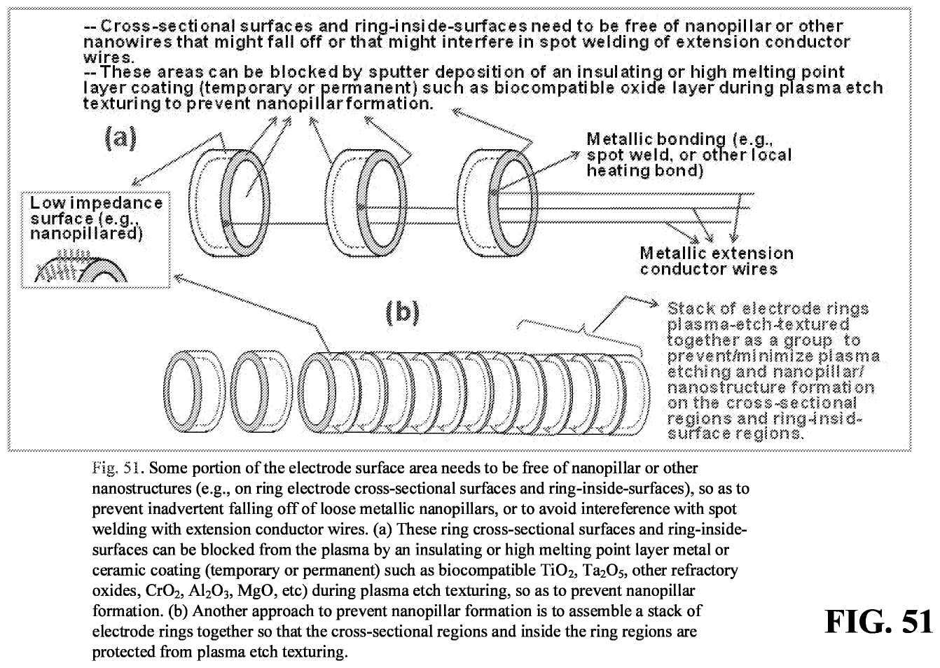

To prevent nanopillar (or nanofeature) formation on ring electrode cross-sectional surfaces and ring-inside-surfaces, these areas can be blocked by an insulating or high melting point layer metal or ceramic coating (temporary or permanent) such as biocompatible TiO₂, Ta₂O₅, other refractory oxides, CrO₂, Al₂O₃, MgO, etc) as a masking layer during plasma etch texturing, as illustrated in FIG. 51(a). These methods to prevent plasma etch and formation of nanopillars or nanostructures in general can also be useful if an array of planar electrodes in square, rectangle or oval form needs to be achieved, e.g. on a paddle electrode array surface.

Yet another approach to prevent nanopillar formation is to assemble a stack of electrode rings together so that the cross-sectional regions and inside the ring regions are not directly exposed to plasma region, and hence are protected from plasma etch texturing, FIG. 51(b).

Illustrated at FIG. 51 is some portion of the electrode surface area that needs to be free of nanopillar or other nanostructures (e.g., on ring electrode cross-sectional surfaces and ring-inside-surfaces), so as to prevent inadvertent falling off of loose metallic nanopillars, or to avoid interference with spot welding with extension conductor wires. (a) These ring cross-sectional surfaces and ring-inside-surfaces can be blocked from the plasma by an insulating or high melting point layer metal or ceramic coating (temporary or permanent) such as biocompatible TiO₂, Ta₂O₅, other refractory oxides, CrO₂, Al₂O₃, MgO, etc) during plasma etch texturing, so as to prevent nanopillar formation. (b) Another approach to prevent nanopillar formation is to assemble a stack of electrode rings together so that the cross-sectional regions and inside the ring regions are protected from plasma etch texturing.

### [HH]. Cap or Sheath Based, Location-Controlled Enhancement of Plasma Etch Texturing.

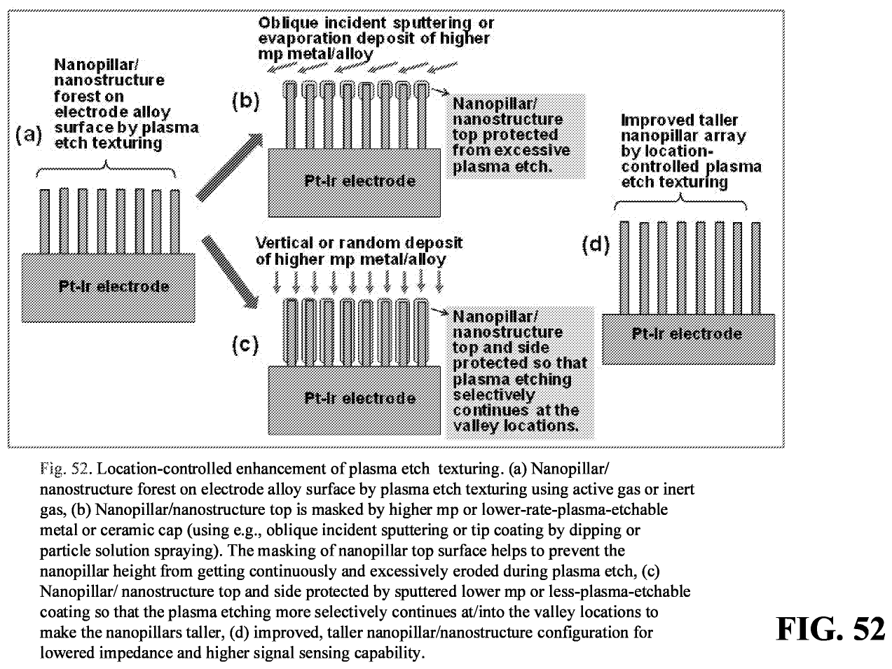

For specific location-controlled enhancement of plasma etch texturing, the invention calls for utilization of Cap or sheath based, suppression of vertical-direction plasma etch, as shown in FIG. 52. The top ends of nanopillar/nanostructure forest on electrode alloy surface by plasma etch texturing (FIG. 52(a)) using active gas or inert gas, are coated with higher mp or lower-rate-plasma-etchable metal or ceramic cap (using e.g., oblique incident sputtering or tip coating by dipping or particle solution spraying followed by annealing to make the tip mask to adhere better), as illustrated in FIG. 52(b). If a much more portion of the nanopillar length can be coated with lower-rate-plasma-etchable metal or ceramic cap (e.g., by using a high-pressure sputtering for deeper plasma penetration toward the nanopillar valley region), FIG. 52(c), most of the nanopillar length can be protected from excessive plasma etch, and hence the continuation of plasma etch would then proceed preferentially from the valley regions, thus resulting in a higher-aspect-ratio nanopillars, FIG. 52(d), for additionally lowered electrode impedance and increased signal sensing capability.

Illustrated at FIG. 52 is a location-controlled enhancement of plasma etch texturing. (a) Nanopillar/nanostructure forest on electrode alloy surface by plasma etch texturing using active gas or inert gas, (b) Nanopillar/nanostructure top is masked by higher mp or lower-rate-plasma-etchable metal or ceramic cap (using e.g., oblique incident sputtering or tip coating by dipping or particle solution spraying). The masking of nanopillar top surface helps to prevent the nanopillar height from getting continuously and excessively eroded during plasma etch, (c) Nanopillar/ nanostructure top and side protected by sputtered lower mp or less-plasma-etchable coating so that the plasma etching more selectively continues at/into the valley locations to make the nanopillars taller, (d) improved, taller nanopillar/nanostructure configuration for lowered impedance and higher signal sensing capability.

### [II]. Hierarchical Nanoporous Surface Coating on Nanopillar Surface for Reduced Impedance and Improved Signal Sensing.

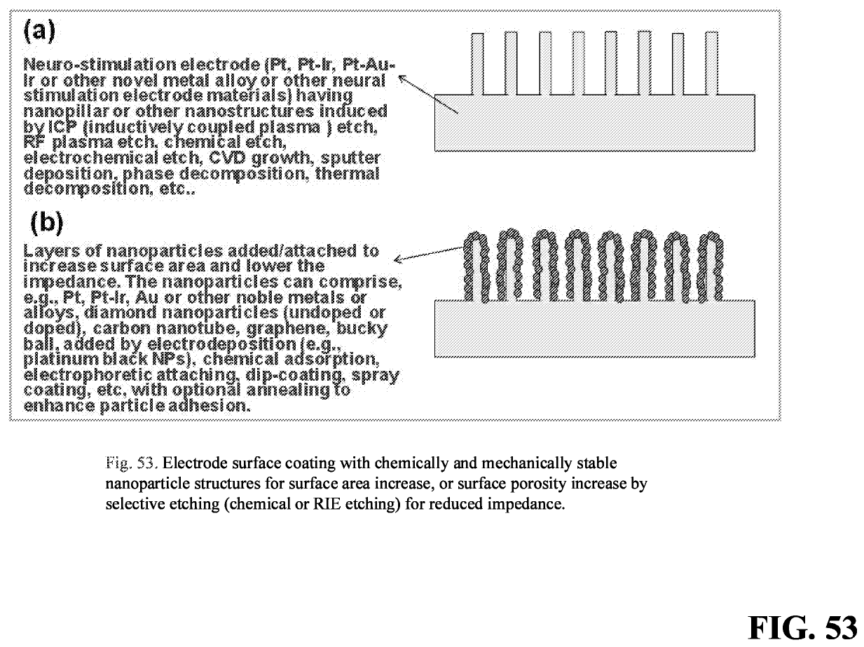

Shown in FIG. 53-FIG. 55 are electrode surface modification methods and structural changes made by deposition of nanoparticles. Described in FIG. 53 is electrode surface coating with chemically and mechanically stable nanoparticle structures for surface area increase, or surface porosity increase by selective etching (chemical or RIE etching) for reduced impedance.

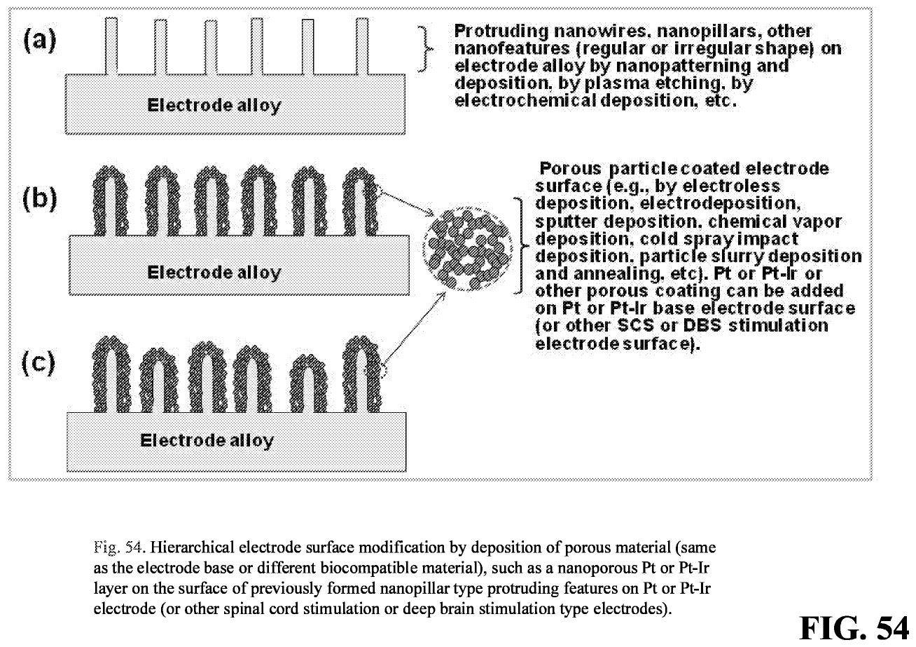

In FIG. 54, a nanoparticle coating which is also nanoporous is illustrated, with a hierarchical electrode surface modification by deposition of porous material (same as the electrode base or different biocompatible material), such as a nanoporous Pt or Pt—Ir layer on the surface of previously formed nanopillar type protruding features on Pt or Pt—Ir electrode (or other spinal cord stimulation or deep brain stimulation type electrodes). Such porous coating can be added by electroless deposition, electrochemical deposition (electrodeposition), physical vapor deposition, chemical vapor deposition, cold spray impact deposition, coating of nanoparticle slurry by spray coating or dip coating followed by optional light sintering at e.g., 200-1000° C. for e.g., 1 min to 10 hrs. For electroless deposition, an example electrolyte solution that can be (HClO₄+K₂PtCl₆) or (cis-dichlorobis(styrene)platinum(II)+toluene).

The particle size that forms the porous layer is nanoscale in nature, typically 1-10 nm, preferably 1-5 nm average diameter. The desired thickness of the porous coating is in the range of 2-50 nm, preferably 5-20 nm. The desired porosity of such added layer is at least 10%, preferably at least 30%, even more preferably at least 50%. The impedance reduction by adding such a porous surface layer is by at least 20%, preferably by at least 40%, even more preferably by at least 60%.

Illustrated at FIG. 53 is an electrode surface coating with chemically and mechanically stable nanoparticle structures for surface area increase, or surface porosity increase by selective etching (chemical or ME etching) for reduced impedance.

Illustrated at FIG. 54 is Hierarchical electrode surface modification by deposition of porous material (same as the electrode base or different biocompatible material), such as a nanoporous Pt or Pt—Ir layer on the surface of previously formed nanopillar type protruding features on Pt or Pt—Ir electrode (or other spinal cord stimulation or deep brain stimulation type electrodes).

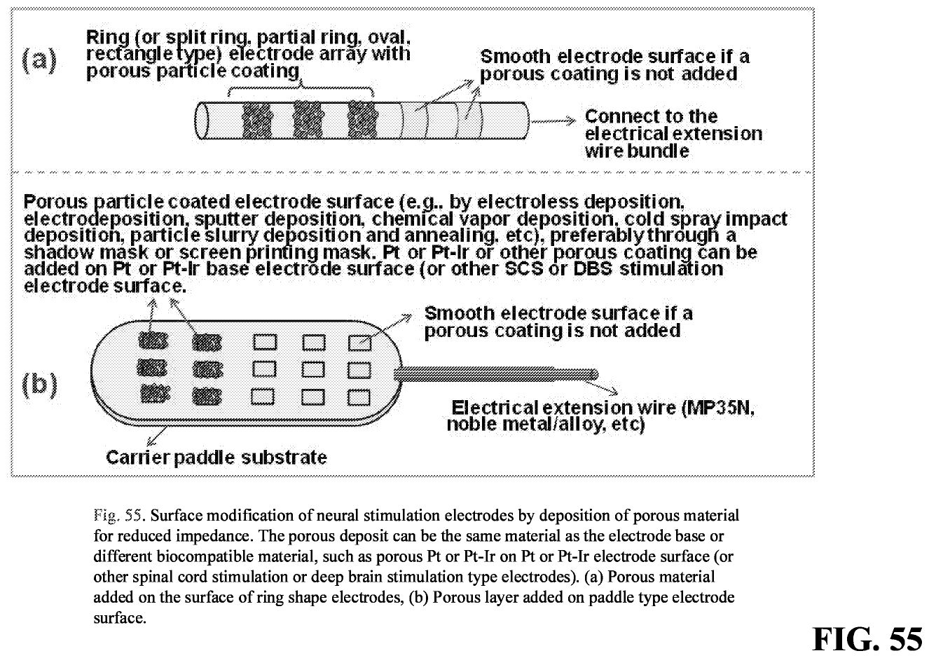

Illustrated at FIG. 55 is a surface modification of neural stimulation electrodes by deposition of porous material for reduced impedance. The porous deposit can be the same material as the electrode base or different biocompatible material, such as porous Pt or Pt—Ir on Pt or Pt—Ir electrode surface (or other spinal cord stimulation or deep brain stimulation type electrodes). (a) Porous material added on the surface of ring shape electrodes, (b) Porous layer added on paddle type electrode surface.

While this invention disclosing document contains many specific details, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

What is claimed is:

1. A neural stimulation system or neural sensing system comprising:
a low-impedance metallic electrode array comprising a surface of nanoscale subdivided structures comprising one or more electrically conducting nanostructure of at least one alignment type;
wherein the at least one alignment type is at least one of a radial alignment, a vertical alignment, a random position, or a partial bridge;
wherein the electrically conducting nanostructure is at least one of a nanowire, nanopillar, nanostructure array, or network nanostructure;
wherein the metallic electrode array comprises a first material;
wherein the metallic electrode array exhibits a reduced impedance by at least 20%, by a factor of at least 2, or by a factor of at least 5 as compared to another electrode comprising a different surface than the surface; wherein the low-impedance metallic electrode array comprises at least one of a spaced-apart circular ring shape, a slitted ring shape, a needle shape, other three-dimensional shape electrodes, a rectangular shape electrode, a square shape electrode, a random shape paddle lead electrodes, or other related electrode configuration; and
a power source component comprising at least one of a battery pack, a power control, or a pulsing control device.

2. The neural stimulation system of claim 1, further comprising anti-biofouling characteristics and a rate of electrical impedance reduction with time decreased by at least 30%, by a factor of two, or at least by a factor of 5 as compared to a different electrode comprising the first material and comprising another different surface than the surface, and absent the anti-biofouling characteristics.

3. The neural stimulation system of claim 1, further comprising an array of base electrodes of ring-like configuration or paddle type electrode carrier with an array of electrodes, or needle or rod shape electrodes,
wherein the surface of the electrode also comprises an array of metallic extension protruding structure including an assembly of microwire or nanowires having mechanically springy and elastically deformable structure, with the microwire having springy properties of being able to tolerate at least 10% compression while still maintaining physical or electrical contacts with the biological surface,
having a microwire or mesh structure with the microwire diameter in the range of 0.1 um to 100 um, preferably 1-50 um,

4. The neural stimulation system of claim 3, wherein the mechanically compliant metallic electrode microwire can be elastically compressed and released (by at least a compression of 20% decreased microwire height) without mechanical breaking failure, to reduce the gap between electrode tip and the tissue or neuro-responsive organ, or to enable direct contact of the microwire tip onto the tissue or organ surface; wherein the gap between the electrode tip and the tissue or neuro-responsive organ is reduced for more powerful electrical pulse amplitude stimulation, with the average gap distance reduction by at least 20%, preferably by at least 50% as compared to the electrode of the same material but without the extended microwire array; wherein the microwire tip region is optionally processed to exhibit low-impedance nanopillar type structure, and wherein the microwire surface is optionally protected by insulating polymer or ceramic coating except the very tip region kept bare for electrical stimulation.

5. The neural stimulation system of claim 3, wherein the mechanically and elastically compressed microwire configuration can be temporarily maintained, either by a layer of sacrificial, dissolvable solid coating, or by tentative confinement of pre-outward-stretched microwire bundle within a guide tube, with the microwire array allowed to be stretched outward by dissolution of the sacrificial solid or by pulling out of the guide tube once the device is inserted into the desired location of human body, so as to contact or almost contact the human body internal surface including spinal epidural space and other surfaces near the neural reception elements, the sacrificial solid polymer or gelatin or food-related material is selected from dried sucrose, gelatin, honey, or other water-soluble polymer or solid which will dissolve with time, that can be programmably set to dissolve after the planned neuro-stimulation implant surgery time period, or any desired time thereafter, so as to release the compressed springy extension microwire electrodes for better physical/electrical contacts with the electrical stimulation or pulsing target locations. Alternatively, the microwire array can also be retained in a compressed state by a tentative confinement of pre-outward-stretched (diameter wise) microwire bundle within a smaller-diameter guide tube, with the microwire array allowed to be released to be expanded/stretched outward for better physical/electrical contacts by removing the guide tube once the device is inserted into the desired location of human body.

6. The neural stimulation system of claim 1, 2 or 3, wherein the selected end portions of the nanopillars or elongated nanostructures are coated with cell-adhesion-resistant or cell-growth-resistant material such as polyethylene glycol (PEG) or PTFE (Teflon), while the remaining lengths of the nanowires are exposed for electrical conduction in the in vivo or in vitro environment so as to impart anti-biofouling, yet allow sufficiently high electrical or ionic conduction for pulse signal to travel to the target location with a sufficient amplitude.

7. The neural stimulation system of claim 1, 2 or 3, wherein the electrode metal is selected from biocompatible metals or alloys including Pt, Pt—Ir, MP35N, noble metals or alloys, stainless steel, Co—Cr alloy or other related alloys.

8. The neural stimulation system of claim 1, 2 or 3, wherein the low-impedance metallic electrode array is processed by:
a first process step comprising a hydrothermal oxide synthesis process followed by an at least partial reduction of the oxide into adhered and protruding metallic nanowires, adhered and protruding nanopillars or a random network structure;
a second process step comprising a reduction treatment in a hydrogen-containing atmosphere to at least partially convert hydrothermally oxide nanostructures into a metallic nanostructure for improved electrical conductivity and adhesion to the base electrode, with a subdivided metallic nanostructure segment having an aspect ratio of at least 3, preferably at least 5, even more preferably at least 10, and the diameter in the preferred range of 50 nm to 500 nm, the nanowire length in the preferred range of 0.2 micrometer to 20 micrometer, with the metal selected from biocompatible metals or alloys including Pt, Pt—Ir, noble metals or alloys, MP35N, stainless steel, Co—Cr alloy or other related alloys, and with the impedance in aqueous solution reduced by at least 50%, preferably by at least a factor of 2.

9. The neural stimulation system of claim 1, 2 or 3, wherein the electrode metal is processed by at least one of the following:
an RF, DC, microwave or inductively coupled plasma exposure process to produce well adhered, protruding metallic nanowires or nanopillars or random network structure, with reactive gas added in the base inert gas by 0-1%, preferably at least 5%, with the base inert gas being argon or other inert gases, and the reactive gas being chlorine or other reactive gases,
a plasma etching process involving the sample temperature to be at room temperature or preferably at 500° C. or higher;
wherein the subdivided nanostructure segment having an aspect ratio of at least 3, preferably at least 5, even more preferably at least 10, and the diameter in the preferred range of 50 nm to 500 nm, the nanowire length in the preferred range of 0.2-20 micrometer; and
wherein the metal selected from biocompatible metals or alloys including Pt, Pt—Ir, noble metals or alloys, MP35N, stainless steel, Co—Cr alloy or other related alloys.

10. The neural stimulation system of claim 1, 2 or 3, wherein an electrode metal of the metallic electrode array is processed by electrochemical deposition growth of nanowires guided by parallel-channeled or radially-channeled membrane including anodized Al₂O₃ membrane or other patterned membrane to produce well adhered, protruding metallic nanowires or nanopillars,
wherein a segment of the nanoscale subdivided structures has an aspect ratio of at least 3, preferably at least 5, even more preferably at least 10, and the diameter in the preferred range of 50 nm to 500 nm,
wherein a nanowire length of the nanoscale subdivided structures is in the preferred range of 0.2 to 20 micrometer,
wherein the nanostructure optionally annealed at high temperature of at least 400° C. for stress relief and/or adhesion improvement by a factor of 2 or higher,
with the metal selected from biocompatible metals or alloys including Pt, Pt—Ir, noble metals or alloys, MP35N, stainless steel, Co—Cr alloy or other related alloys,
with the preferred Pt—Ir composition range of 5-40% Ir, preferably 10-20% Ir.
Alternatively, pure Pt nanowires can be grown, with Ir film sputter coated, followed by annealing to diffuse Ir into the Pt matrix, to at least form Pt—Ir alloy skin surface, or Ir oxide skin surface can be produced.

11. The neural stimulation system of claim 1, 2 or 3, wherein the electrode metal is processed;
by nanopatterning using e-beam lithography, nanoimprint lithography, deep UV lithography, extreme UV lithography or variations/combinations of these processes utilizing resist layer materials, with optional deposition of electrode alloy nanowires into patterned channels or modified configuration to produce well adhered, periodically or randomly positioned, metallic nanowires or nanopillars or random network structure,
with an optional high pressure Ar based sputtering deposition of electrode material into the nanopatterned channels or nanopatterned holes for deeper penetration and higher-aspect-ration protruding structures with a benefit of further reduced impedance,
with another option of pre-depositing mask islands so as to form a protruding nanopillars by RIE etching except the masked islands,
with the subdivided nanostructure segment having an aspect ratio of at least 3, preferably at least 5, even more preferably at least 10, and the diameter in the preferred range of 50 nm to 500 nm, the nanowire length in the preferred range of 0.2-20 micrometer,
with the metal selected from biocompatible metals or alloys including Pt, Pt—Ir, noble metals or alloys, MP35N, stainless steel, Co—Cr alloy or other related alloys.

12. The neural stimulation system of claim 1, further comprising by using electroplating or guided electroplating on previously grown shorter nanowire or nanopillar seeds,
with the previously grown nanowires or nanopillars prepared by hydrothermal growth of oxide nanowires followed by reduction, prepared by electrodeposition through a mask, prepared by RF, DC, microwave, or ICP plasma etching steps, or prepared by nanopatterning aided by patterned resist layer,
with the increase in nanowire or nanopillar length being at least 30%, preferably by at least 100% of the previously grown seed nanowire or nanopillar length,
with the impedance further reduced by at least 10%, preferably 30%, more preferably 100% through such additional extension of nanostructure length.

13. The neural stimulation system of claim 1, 2 or 3,
wherein the said metallic electrode matrix is a composite electrode comprising electrode alloy phase and oxide or other ceramic phase,
wherein the metallic alloy phase is selected from Pt, Pt—Ir, noble metals or alloys, MP35N, stainless steel, Co—Cr alloy or other related alloys, and where the ceramic phase is selected from oxides such as TiO₂, Ta₂O₅, ZrO₂, Al₂O₃, SiO₂, from nitrides such as Si₃N₄, AlN, BN, TiN, TaN, ZrN, or fluorides or carbides,
wherein the grain size is reduced at least by a factor of two as compared with the nanowire or nanopillar without the composite structure,
wherein the electrical resistivity of the composite part of the nanowires or nanopillars is increased at least by 50%, preferably at least by a factor of 2 as compared with the base nanowire or nanopillar without the composite structure.

14. The neural stimulation system of claim 1, 2 or 3,
wherein the said metallic electrode is further coated with high resistivity, fine grain size electrode alloy selected from Pt, Pt—Ir, noble metals or alloys, MP35N, stainless steel, Co—Cr alloy or other related alloys,
with the grain size of the deposited coating layer electrode alloy being smaller than 100 nm, preferably less than 20 nm, even more preferably less than 5 nm,
with the electrical resistivity of the coated metallic layer increased at least by 50%, preferably at least by a factor of 2 as compared with the base nanowire or nanopillar material.

15. The neural stimulation system of claim 1, 2 or 3, wherein the metallic electrode is coated with high resistivity, fine grain size electrode alloy selected from a group of Pt, Pt—Ir, noble metals or alloys, MP35N, stainless steel, Co—Cr alloy or other related alloys, wherein the coating comprises a composite material comprising an electrode alloy phase and an oxide or other ceramic phase, and wherein the electrical resistivity of the coated part of the nanowire or nanopillar is increased at least by 50%, preferably at least by a factor of 2 as compared with the base nanowire or nanopillar.

16. The neural stimulation system of claim 1, 2 or 3, wherein the metallic electrode array is further coated with high resistivity, fine grain size electrode alloy selected from Pt, Pt—Ir, noble metals or alloys, MP35N, stainless steel, Co—Cr alloy or other related alloys, wherein the grain size of the deposited coating layer electrode alloy being smaller than 100 nm, preferably less than 20 nm, even more preferably less than 5 nm.

17. The neural stimulation system of claim 1, 2 or 3, wherein the metallic electrode is a coated metal or alloy layer on non-metallic nanowires, nanopillars or sharp needles made of Si, oxide, nitride, carbide, carbon nanotube, or composite ceramics, or polymer needles, by using deposition techniques including sputtering, evaporation, e-beam or laser ablation deposition, CVD deposition, electroless coating or electrodeposition.

18. The neural stimulation system of claim 1, 2 or 3, wherein the said metallic electrode has a partially coated insulator material at the lower portion of the equi-diameter or taper-sharpened nanowires or nanopillars so as to enable focusing of the electrical pulse signals.

20. The neural stimulation system of claim 1, 2 or 3, wherein the electrode metal tip is coated with Au, pd, Pt, or other noble metals or alloys,
for improved corrosion resistance and reduced biofouling to enable at least by a factor of two longer usage for the similar degree of electrode performance deterioration,
with an optional adhesion layer such as Ti, Zr, Hf, Ta, Cr,Al at the interface for stronger adhesion of the noble metal tip nanoporous with enhanced surface area and further reduced electric impedance.

20. The neural stimulation system of claim 1, 2, or 3 comprising the low-impedance metallic electrode, wherein the electrode or an array of electrode is used for electrical stimulation of neural activity for health benefit of human or animal body,

21. The neural stimulation system of claim 1, 2, or 3 comprising the low-impedance metallic electrode of claim 1, 2, or 3,
wherein the electrode or an array of electrode is used for measurement and monitoring of human or animal body functioning involving neural signals for diagnostic purpose or for monitoring purpose, including brain activities, spinal cord pain reduction response recording, heart functions, or feedback-based pulsing to ease the pain, including the use of electrically evoked compound action potential (ECAP) signals,
wherein the nanostructured stimulation electrode of the present invention desirably provides at least 50% increased sense signal (in peak current amplitude), preferably at least 100% increased signals, more preferably at least 200% increased signals as compared to the identical sized electrode material with non-textured smooth surface.

22. The neural stimulation system of claim 1, 2, or 3 comprising the low-impedance metallic electrode of claim 1, 2, or 3, wherein the electrode or an array of electrode is used for study and control of brain functions or other human/animal body functions including cell behavior, organ behavior, blood-related, diabetes related, glucose monitoring behavior, heart related, hormone related monitoring/control, and other related purposes.

23. The neural stimulation system of claim 22 comprising an electrode lead and electrode extension with the subdivided structure,
having structurally subdivided electrode lead wires with higher electrical resistance by at least 20%,
having more advantageous response of reduced eddy current, reduced heating and battery energy savings on higher frequency electrical stimulation. Optional annealing heat treatment can be utilized for intermediate softening or better bonding between adjacent subdivided wires.

24. The neural stimulation system of claim 22, wherein the subdivided electrode lead and the electrode extension are selected from multifilamentary subdivided leads or phase-elongated subdivided leads for higher frequency operation,
with the operating frequency being able to be increased at least by a factor of two.

25. The neural stimulation system of claims 1-24, wherein the operable frequency range of the electrode pulses of electrode structures and materials is increased at least by a factor of two, preferably by a factor of 5.

26. The neural stimulation system of claims 1, 2 or 3, wherein the electrodes can perform drug delivery functions from the presence of a drug-absorbable forest of impedance-lowering nanopillar type structure, including drugs selected from antibiotics, steroids, immuno-modulator drugs, hormones, small molecule drugs, or other therapeutic drugs.

27. The neural stimulation system of claim 1, 2 or 3, wherein the electrodes can perform slow, time-dependent drug delivery functions from the impedance-lowering nanopillar type structure, with the controlled drug release speed controlled by dissolution speed of a sacrificial coverage material such as solid polymers selected from dried sucrose, gelatin, honey, or other water-soluble polymer or compound which can be programmably set to dissolve after the planned surgery time period, or any desired time,
with the drug-releasing material trapped in the nanopillar forest,
with the thickness of the sacrificial coverage material, the nature and porosity of the material adjustable,
with the nanopillar density on the electrode surface adjustable,
with the viscosity of the impregnated drug in the nanopillar forest adjustable.

28. The neural stimulation system of claim 1, 2 or 3, wherein the nanopillar or related nanostrucutres are mechanically safe-guarded by adding one or more protective shoulder structure to mechanically shield the nanopillar type, impedance-lowering structures during assembly, handling, shipping, implanting operations.

29. The neural stimulation system of claim 28, wherein the protective shoulder can be fabricated by;
machining, etching, metal press-forming, or by additive manufacturing,
with the shoulder made of the same ring or electrode material or other material,
with the nanopillar type, impedance lowering structure on the shoulder optionally removed if desired (e.g., by polishing or etching away). Alternatively, the shoulder surface can be masked to prevent nanopillar formation during the plasma or electrochemical processing.

30. The neural stimulation system of claim 1, 2, or 3, wherein manufacturing of ring electrodes (closed ring or split ring) with low impedance surface can be carried out by;
(i) plasma surface texturing to form nanopillar surface structure, (ii) chemical etching, (iii) anodization, (iv) electrochemical deposition of radial nanopillars.

31. The neural stimulation system of claim 30, wherein the nanopillar forming processing can be performed with;
(a) a long cylinder first which is then sliced into short width ring electrodes, or
(b) processing or a stacked short rings followed by separation, or
(c) processing of flat strips followed by bending/curbing into a ring configuration. Some shoulder structure can optionally be added near the edge of the strips so that the nanopliiars are not mechanically damaged during bending operation or other mechanical shaping, or during handling.

32. Systems, devices, electrode structures and materials of claims 1-31 wherein the applications of the low impedance, anti-biofouling electrode include medical implant neural stimulator devices, neural diagnosis tools, spinal cord and peripheral nerve stimulation, deep brain stimulation, and cochlear implants, treatment of Alzheimer's Disease, Parkinson's Disease, heart disease, hearing loss and head trauma, epilepsy, and so forth.

33. Systems, devices, electrode structures and materials of claim 32,
wherein the neural stimulation includes spinal cord stimulation that can utilize both low frequency regime stimulation, BURST, intra and inter BURST, noise, as well as high frequency regime cord stimulation ranging from 0-100,000 Hz methods for reducing chronic or transient pains, with or without, or with reduced paresthesia such as an abnormal sensation of tingling, pricking or numbness,
with the substantially reduced impedance allowing advantageous neural stimulations using altered or higher-amplitude pulse waves or a train of pulse wave forms for medical benefits,
the spinal neural stimulation electrode array in the form of leads is positioned in the epidural space above the spinal cord to deliver electrical current to the area of pain.

34. Systems, devices, electrode structures and materials of claims 1-33,
wherein the need for battery power in the implant system is reduced because of the lowered impedance to a decreased level at least by a factor of 50%, preferably by a factor of 2, more preferably by a factor of 5, even more preferably by a factor of 10.

35. Systems, devices, electrode structures and materials of claims 1-33,
wherein the physical size of the implanted battery is reduced at least by a factor of 50%, preferably by a factor of 2, more preferably by a factor of 5, even more preferably by a factor of 10, as compared to the electrodes without the impedance reducing structure.

36. Systems, devices, electrode structures and materials of claims 1-33,
wherein the shape of the implanted battery is altered from a bulky configuration into a linearly positioned series of batteries having an appearance of small diameter lead wire shape,
with the diameter of the lead wire shaped battery is less than 2 mm, preferably less than 1.5 mm, even more preferably less than 1 mm.

37. Systems, devices, electrode structures and materials of claims 1-33,
wherein the reduced size of the implanted battery enables a single incision implanting operation instead of two incisions of inserting the electrode lead(s) to the epidural space and inserting the battery with control console electronics near the hip cavity.

38. Systems, devices, electrode structures and materials of claims 1-33 or other structures that allow feedback-controlled neural stimulation for pain reduction or body function control, utilizing body-response-electrical-signals as a convenient means to adjust or modify subsequent electrical pulsing intensity and mode for optimized neural stimulation.

39. Systems, devices, electrode structures and materials of claims 1-33 wherein the electrical power needed is at least partially supplied by human body generated electricity such as enzymatic biofuel cell or glucose based biofuel cells for power generation, thermoelectric power generation utilizing temperature gradient or temperature difference between different parts of human body, or use of body motion (e.g., walking) utilizing piezoelectric generator or electromagnetic power generation (e.g., walking motion inducing movement of magnetic component near solenoid array). The human-body-generated electricity can be stored in the implanted battery for use in a convenient manner.

40. A method of scaled up manufacturing of nanopillars or nanopores described in claims 1-33 wherein continual or continuous chemical or electrochemical deposition is carried out,

41. A method of scaled up manufacturing of nanopillars or nanopores described in claims 1-33, by continual or continuous electrochemical etching of metallic alloys of neuro-stimulation electrode material.

42. A method of scaled up manufacturing of nanopillars or nanopores by continual or continuous plasma process of feeding and optionally taking up into would up materials storage mode, wherein.
the plasma process is optionally performed in multiple steps to further elongate the nanopillar aspect ratio,
the plasma process is optionally performed in active gas such as chlorine or fluorine, or alternatively using inert gas plasma in multiple steps.

43. A lowered impedance electrode alloy apparatus for neuro-stimulation by deposited particles of noble metal or alloy through electrodeposition or chemical deposition or electrophoretic deposition of nanoparticle alloys such as Pt, Pt—Ir, Pt—Au—Ir or other noble metal alloys, followed by optional annealing for stress relief and enhanced adhesion.

44. The neural stimulation system of claim 1, 2, or 3, with the electrode impedance is further lowered by deposited microparticles or nanoparticles of noble metal or alloy on the electrode surface, wherein;
the particles are deposited by sputtering, evaporation, electrodeposition or electroless chemical deposition, electrophoretic deposition, wet spray deposition, cold spray or plasma spray impact deposition, or dip-coating of nanoparticles of alloys such as Pt, Pt—Ir, Pt—Au—Ir or other noble metal alloys,
with such particles deposited on either smooth-surfaced or nano- or micro-pillar-structured surface,
with the nano- or micro-pillar-structured surface prepared by ICP plasma etch, RF, DC, microwave plasma etch, nanopatterning, deposition through vertical pores, or through anodized template hole array,
with the particle-deposited structure optionally annealed at high temperature for stress relief and for enhanced particle adhesion.

45. The neural stimulation system of claim 1, 2, or 3, with the electrode impedance is further lowered by deposited microparticles or nanoparticles of noble metal or alloy on the electrode surface, wherein;
the deposited particles are selected to be 0.5-10 nm average diameter, preferably 1-5 nm,
the porosity is controlled to be at least 10%, preferably at least 30%, even more preferably at least 50%,
the desired thickness of the porous coating is in the range of 2-50 nm, preferably 5-20 nm,
the impedance reduction by adding such a porous surface layer is at least 20%, preferably by at least 40%, even more preferably by at least 60%.

46. The neural stimulation metallic electrode system of claim 45, wherein;
the electroless deposition is carried out using electrolyte solutions including (HClO₄+K₂PtCl₆) or (cis-dichlorobis(styrene)platinum(II)+toluene) solution.

47. A lowered impedance electrode alloy for neuro-stimulation by chemical or electrochemical etching of two-phase or multi-phase alloy or dealloying of alloys such as Pt, Pt—Ir, Pt—Au—Ir or other noble metal alloys, using a strong acid or other chemicals on the surface of nanopillar or micropillar array prepared by ICP plasma etch, RF plasma etch, nanopatterning, deposition through vertical pores, anodization. The surface area of the nanopillar is improved by at least 30%, preferably 50% by such nanopore etching.

48. A neural stimulation electrode structure comprising anti-biofouling coating applied onto local regions of nanostructure top surface such as the tip of nanopillars, with the anti-biofouling agent selected from PEG, PEGlated polymer, OEG (of oligo-ethylene glycol), triblock-copolymer loop, fluoropolymer, Perfluoropolyether-based random terpolymers, Zwitterionic polymers (e.g., phosphatidylcholines), oligosaccharide grafted polymers mimicing the antifouling glycocalyx, polyoxazoline polymers (e.g., comb polymers with poly (2-methyl-2-oxazoline) (PMOXA) side chains and a polycationic poly(L-lysine) (PLL) backbone, diamond, PVDF (polyvinylidene difluoride) or other fluoropolymer or carbon-fluorine compound.

49. A low impedance neuro-stimulating electrode apparatus which, in a simulated pseudo-physiological environment (e.g., tissue/fat/blood mixed environment), exhibits impedance reduction by nanopillar electrodes is still maintained, with high frequency stimulation at 1 KHz or higher, with the pseudo-physiological environment making the nanopillar electrode exhibit more attractive lower impedance than the regular non-textured electrode. In addition, for higher frequency of 100 KHz to 1 MHz, the nanopillar electrode exhibits in the pseudo-physiological environment, much improved lower impedance than in the PBS solution by at least 50% more reduction in impedance, up to a high frequency pulse operation as high as 2 MHz.

50. A method of preparing a low impedance neuro-stimulating electrode by utilizing a template nanopillar or related nanostructure of metal, oxide or nitride ceramic, carbon nanotube or nanocone, onto which biocompatible and low-impedance Pt or Pt—Ir or noble metal is coating-deposited (e.g., by sputtering, evaporation, electrodeposition) so as to maintain and utilize the previously protruding nanostructured template (e.g., plasma textured MP35N or electrodeposition prepared, radially aligned Ni nanowire array, carbon nanotube or nanocone) for reduced impedance.

51. A method of preparing a low impedance neuro-stimulating electrode by;
utilizing a well texturing sacrificial coating material (layer 1 material) on the surface of intended electrode material (layer 2 material) to form a nanopillar or related nanostructure, then continuing plasma texturing so that the nanopillar structure pattern formed on the coating material is eventually transferred to the electrode material underneath upon continued plasma processing.

52. A method of preparing a low impedance neuro-stimulating electrode as described in claim 48, wherein;
the sacrificial Layer 1 coating material is Nichrome alloy, MP35N alloy, or other Cr-, Ni- or refractory-metal-containing alloy, and
the Layer 2 substrate material is Pt—Ir base or Pt-base alloys.

53. A structure of IrO₂ surface layer added onto nanopillar-structured Pt—Ir, MP35N or other biocompatible electrode alloy surface to reduce the impedance by at least 30%, preferably at least by a factor or 2.

54. A method of producing impedance lowered, IrO₂ surface coated nanopillar electrode;
by intentional oxidizing by heat treatment of Pt—Ir electrode at e.g., 300-700° C. for 0.5 to 5 hrs so as to form a thin IrO₂ layer of 1-100 nm, preferably 5-50 nm, or
by sputter coating of thin Ir layer on electrode surface followed by intentional oxidation heat treatment, or
by direct deposition and coating of electrode surface by deposition of IrO₂ by e.g., RF sputtering, or
by ion implantation of Ir followed by surface oxidation or Ir and oxygen ion implantation.

55. A method of preparing a low impedance neuro-stimulating electrode by; by hydrothermal process on biocompatible electrode alloy base (e.g., Pt, Pt—Ir, MP35N, and so forth) in wire shape, ribbon shape or in plate shape, utilizing a processing steps of;
placing the base electrode or assembly of electrode in an autoclave vessel to grow oxide nanopillar array (e.g., Co-oxide, Ni-oxide, Ti-oxide, refractive metal oxide, alloy oxide, in the form of nanopillars, nanowires, nanoribbons or other protruding nanostructures) in a salt solution at >100° C.), to radially grow nanopillars or related nanostructures on wire shape substrate surface, to vertically grow nanopillars or other nanostructures on ribbon-shape or plate-shape substrate,
with the desired nanopillars or similar structures in the dimension of 20-1,000 nm in average diameter (preferably 50-200 nm), having an aspect ratio of e.g., ˜3-50, preferably 5-20,
the surface of the hydrothermally grown oxide nanopillar are coated a biocompatible electrode alloy metal (e.g., Pt, Pt—Ir, Au, their alloys, MP35N), e.g., −20-50 nm thick, with an optional adhesion layer of 2-5 nm thick Ti, Zr, Ta, deposited in-between, using sputter-coating, evaporation coating, chemical or electrochemical coating, either before oxide-reduction step or after the oxide-reduction step,
apply an oxide reduction heat treatment to reduce and convert the oxide core to metallic material by H₂ gas atmosphere reduction or hydrogen-containing atmosphere at high temperature of 300-1000° C. for 10 min to 24 hrs, which also enhances adhesion of nanopillars to the base electrode alloy, and that of Pt, Pt—Ir, MP35N coated metal layer onto nanopillar surface,
with an optional switching of processing sequence of performing the reduction heat treatment of oxide nanopillars to metallic nanopillars first before the sputter deposition.

56. A method of manufacturing one or more low impedance alloy utilized for deep brain stimulation or other neural stimulation, or other feedback-based neural stimulation comprising:
using either a same pulse stimulating electrode employing on time delay effect of captured ECAP signal as compared to the pulsing timing to manufacture the one or more low impedance alloy, or
using a separate set of dedicated sensing electrodes for signal pick up for feedback controlled modified pulsing to manufacture the one or more low impedance alloy.

57. The neural stimulation system of claims 1 to 3, wherein chemical or electrochemical pre-etch treatment is used to produce initial surface cavities or etch pits to make the subsequent nanopillar formation easier during plasma etch process to obtain at least 10% reduced impedance and at least 10% improved signal sensing capability.

58. The neural stimulation system of claims 1 to 3, wherein island array masks are provided via high melting point metal/alloy island deposition using sputtering, electrodeposition, etc, optionally using nanotemplates such as anodized aluminum oxide (AAO) membranes or block copolymer (BCP) membranes.

59. The neural stimulation system of claims 1 to 3, wherein pre-treatment modification of previously plasma textured electrode surface by mechanical, chemical, electrochemical, reactive ion removal of existing nanopillar type structures, is followed by second plasma etch texturing for higher density, taller and more uniform nanopillar structures.

60. The neural stimulation system of claims 1 to 3, wherein a nano membrane/mask is pre-deposited to allow a subtractive process of making selective local surface pitting through the open regions of the membrane/mask.

61. The neural stimulation system of claims 1 to 3, wherein a nano membrane/mask is pre-deposited to produce selective local surface nano-protrusions to serve as guiding feature or nuclei feature for subsequent plasma etch texturing. The protrusion can be made by sputter deposition, evaporation, CVD, electrodeposition of either an identical material as the electrode (e.g., Pt—Ir alloy), or a different material (e.g., high mp metal/alloy or ceramic material protruding mask).

62. The neural stimulation electrode system of claims 1 to 3, wherein plastic and elastic deformation of nanopillars and associated nanogeometry is obtained by drawing the electrode wire through a die, rolling deformation of a strip of electrode paddle, contact sliding, contact rotating deformation, etc to bend nanopillar type structures, so as to expose previously hidden substrate regions (by nanopillar forest) for additional plasma etch, so as to contribute to lowered impedance and increased sensing signals.

63. The neural stimulation system of claims 1 to 3, wherein the formation of nanopillar or other nanostructures (e.g., on ring electrode cross-sectional surfaces and ring-inside-surfaces) is intentionally prevented by coating of an insulating or high melting point layer metal/alloy or ceramic coating (temporary or permanent) such as biocompatible TiO₂, Ta₂O₅, other refractory oxides, CrO₂, Al₂O₃, MgO, etc) during plasma etch texturing, so as to prevent nanopillar formation. Another approach to prevent nanopillar formation is to assemble a stack of electrode rings together so that the cross-sectional regions and inside the ring regions are protected from plasma etch texturing.

64. The neural stimulation system of claims 1 to 3, wherein location-controlled enhancement of plasma etch texturing is achieved by masking of nanopillar/nanostructure top or side wall by higher mp or lower-rate-plasma-etchable metal or ceramic cap coating so that the plasma etching more selectively continues at/into the valley locations to make the nanopillars taller, with lowered impedance and higher signal sensing capability.

65. The neural stimulation system of claims 1 to 3, wherein;
the nanopillar or nanowire configuration on the electrode surface is protected during surgery on insertion to the epidural space by providing geometrically recessed configuration so that the nanopillar type structure is not scraped off during insertion, or
a temporarily protective coating is applied onto the electrode surface to cover up the nanostructures during insertion to epidural space, with the protective coating material later dissolved away inside human body,
with such as biocompatible and dissolvable material selected from gelatin, starch, syrup, honey, hydrogel and other dissolvable materials.

66. A method of improving a neural stimulation system comprising:
utilizing a plastic and elastic deformation of nanopillars and associated nanogeometry to bend one or more nanopillar type structure resulting in an exposure of previously hidden substrate regions, by a nanopillar forest, wherein the previously hidden substrate regions are accessible for one or more additional plasma etch subsequent to an initial plasma etch, wherein the one or more nanopillar type structures have a higher density as compared to a density prior to the plastic and elastic deformation, and wherein the higher density of the one or more nanopillar type structures results in the one or more nanopillar type structures having a lower impedance and increased sensing signal by at least 10% and preferably at least by 30%, and wherein a sensing signal is at least one of an ECAP type signal.

67. The method of claim 66, further comprising:
utilizing a location-controlled enhancement of a plasma etch texturing process to mask the nanopillar type structure top by higher melting temperature or lower-rate-plasma-etchable metal or ceramic cap, optionally using an oblique incident sputtering or tip coating by dipping or particle solution spraying,
wherein the masking of nanopillar top surface helps to prevent the nanopillar height from getting continuously and excessively eroded during plasma etch,
wherein the nanopillar or nanostructure top and side are protected by sputtered less-plasma-etchable coating so that the plasma etching more selectively continues at/into the valley locations to make the nanopillars taller, and
wherein the improved, taller nanopillar/nanostructure configuration exhibiting lowered impedance and higher signal sensing capability by at least 10%, preferably at least 30%, even more preferably at least by a factor of 2.
